(19) United States
(12) Patent Application Publication
(10) Pub. No.: US 2011/0230426 A1
(43) Pub. Date: Sep. 22, 2011

(54) TEMPLATE-FIXED PEPTIDOMIMETICS

(75) Inventors: Daniel Obrecht, Battwil (CH); Frank Gombert, Binningen (CH); Markus Seitz, Zurich (CH); Ludovic T. Maillard, St. Jean de Bueges (FR); Roba Mounme, Zurich (CH); Kerstin Moehle, Wettswil (CH); John Anthony Robinson, Wermatswil (CH); Odile Sellier-Kessler, Baldersheim (FR); Heiko Henze, Zurich (CH); Christian Bisang, Basel (CH); Alexander Lederer, Basel (CH)

(73) Assignees: POLYPHOR AG, Allschwil (CH); UNIVERSITAT ZURICH, Zurich (CH)

(21) Appl. No.: 13/057,932

(22) PCT Filed: Aug. 8, 2008
(86) PCT No.: PCT/EP08/60494

§ 371 Date: Jun. 7, 2011

(51) Int. Cl. A61K 38/12; C07K 7/64; C07K 1/00; A61P 3/10; A61P 29/00; A61P 13/12; A61P 9/00; A61P 11/06; A61P 11/02; A61P 11/00

(52) U.S. Cl. 514/21.1; 530/321

(57) ABSTRACT

Template-fixed β-hairpin peptidomimetics of the general formula

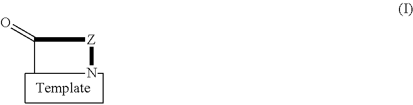

wherein Z is a template-fixed chain of 8 α-amino acid residues which, depending on their positions in the chain (counted starting from the N-terminal amino acid), are Gly or Pro or of certain types which, as the remaining symbols in the above formula, are defined in the description and the claims, and salts thereof, have agonizing or antagonizing activity against urotensin II or show inhibition of the STAT6/NCoA-1 interaction and can be used for preventing or treating diseases or disorders related to urotensin II, STAT6 and NCoA-1.

These β-hairpin peptidomimetics can be manufactured by a process which is based on a mixed solid- and solution phase synthetic strategy.

The present invention provides template-fixed β-hairpin peptidomimetics incorporating a template-fixed chain of 8 α-amino acid residues which, depending on their positions in the chain, are Gly or Pro or of certain types, as defined herein below. These template-fixed β-hairpin mimetics have an agonizing or antagonizing activity against urotensin II, a G-protein-coupled receptor (GPCR), or show inhibition of the STAT6/NCoA-1 interaction, wherein STAT6 is a transcription factor of the STAT family and NCoA-1 a transcriptional coactivator, also called SRC-1. In addition, the present invention provides an efficient synthetic process by which these compounds can, if desired, be made in parallel library-format.

Many medically significant biological processes are mediated by signal transduction that involves GPCRs. The family of GPCRs includes receptors for hormones, neurotransmitters, growth factors and viruses (Th. Klabunde, G. Hessler, ChemBioChem 2002 3, 928-44). As for 210 receptors the natural ligand is known, another 150, so-called orphan receptors, have been identified within the human genome, for which the (patho)physiological function is unknown (A. Wise, S. C. Jupe, S. Rees, Annu. Rev. Pharmacol. Toxicol. 2004, 44, 43-66).

The GPCRs can be grouped into three major families: family A (rhodopsin-like or adrenergic-like family), family B (glucagon-receptor-like or secretin-receptor-like family) and family C (metabotropic glutamate receptors). Within each receptor family a certain sequence pattern (so-called fingerprint) and several structural features beyond the generally shared membrane topology are conserved (T. K. Attwood, Trends Pharmacol. Sci. 2001, 22, 165-65). Family A is by far the largest class. GPCRs are membrane-bound and characterized by a conserved seven helix transmembrane-spanning domain. As the first GPCR structure at atomic resolution, the 3D structure of bovine rhodopsin by X-ray crystallography was reported (K. Palczewsky et al. Science 2000, 289, 739-45). Based on this structure several models for other GPCRs have been reported using homology modeling (M. C. Gershengorn et al. Endocrinology 2001, 142, 2-10; S. Shacham et al. Med. Res. Rev. 2001, 21, 472-83). Recently, the crystal structure of the human β₂-adrenergic GPCR has been published (S. G. Rasmussen et al. Nature 2007, 450, 383-387).

Although over the past 15 years, nearly 350 therapeutic agents targeting GPCR receptors have been successfully introduced into the market (Th. Klabunde, G. Hessler, ChemBioChem 2002, 3, 928-44; G. Vauquelin et al. Fundam. Clin. Pharmacol. 2005, 19, 45-56), several toxicological problems which arose from mainly lack of selectivity of some of those drugs, need to be further investigated. Clearly there is a need for new compounds for treating or preventing diseases including, but not limited to, infections, cancers, allergies, cardiovascular and peripheral and central nervous system disorder.

Transcription factors are central mediators of signal transduction. Manipulation of their activity by small molecules is a rapidly emerging area of both chemical biology and drug discovery (D. Ghosh, A. G. Papavassiliou, Curr. Med. Chem. 2005, 12, 691). One class of transcription factors contains signal transducer and activator of transcription (STAT) proteins, involved in many biological and medical relevant events, e.g. programmed cell death, organogenesis, innate and adaptive immunity or cell growth regulation (C. M. Horvath, TiBS, 2000, 25, 496). Transcription factors perform their function alone or by recruiting components of the transcription machinery to activate transcription. One type of these components are transcriptional coactivators.

Many drugs exert their effects through transcription factors whereof approximately 900 are associated with known diseases. Although transcription factors are key players in the pathogenesis of disease the complexity of the biology of transcriptional regulation still presents challenges to the discovery of new drugs as well as the design of therapies that directly target molecules involved in the transcription process. As the specificity of modulators plays a crucial role within therapeutic interventions as well there is clearly a need for new compounds for treating or preventing diseases including, but not limited to, various cancer like acute promyelocytic leukemia, breast cancer, endometrial cancer, prostate cancer, heptacellular carcinoma, metastasis, autoimmune diseases like airway hyperresponsiveness (AHR), eosinophilic inflammation, mucus production, asthma, neurodegenerative diseases, restinosis and gastrointestinal nematode parasites.

The present invention describes a novel general approach to discover potent, selective and druggable ligands for GPCRs and modulators of transcriptional factors and coactivators. Within the scope of the present invention, this approach is particularly suited to discover ligands for peptidergic GPCRs as well as transcriptional coactivators.

Some of the peptidergic GPCR ligands/receptors interactions that are of therapeutic relevance are:

Somatostatins (A. V. Schally et al. Cell. Mol. Life. Sci. 2004, 61, 1042-68), neurokinins, neurotensins (W. Rostène et al. Encyclop. Biol. Chem. 2004, 3, 3236; M. Boules et al. Expert. Opin. Investig. Drugs 2005, 14, 359-69; P. Kitabgi, Curr. Opin. Drug Disc. Devel. 2002, 5, 764-76), bradykinins (F. Marceau et al. Nat. Rev. Drug Disc. 2004, 3, 845-52), vasopressins (M. Ashton et al. Comb. Chem. And High Throughput Screening 2004, 7, 441-53), tachykinins, bombesins (E. R. Spindel et al. Recent Progress in Hormone Research 1993, 48, 365-91; R. T. Jensen et al. Growth Factors, Peptides, and Receptors, p. 225-237, Ed. By T. W. Moody, Plenum Press, New York, 1993; A. V. Schally et al. Cell. Mol. Life. Sci. 2004, 61, 1042-68), endothelins (G. Ertl et al. Drugs 2004, 64, 1029-40), urotensin II (F. D. Russell, Pharmacol. Ther. 2004, 103, 223-43), GH-RH (A. V. Schally et al. Cell. Mol. Life. Sci. 2004, 61, 1042-68), ghrelin (A. V. Schally et al. Cell. Mol. Life. Sci. 2004, 61, 1042-68; E. Ghio et al. Clin. Endocrinol. 2005, 62, 1-17), melanocortins (B. G. Irani et al. Curr. Pharm. Des. 2004, 10, 3443-79), glucagon-like peptide 1 (GLP-1, C. J. Small et al. Curr. Drug Targets CNS Neurol. Disord. 2004, 3, 379-88), peptide YY (PYY, C. J. Small et al. Curr. Drug Targets CNS Neurol. Disord. 2004, 3, 379-88), VIP (A. V. Schally et al. Cell. Mol. Life. Sci. 2004, 61, 1042-68), and protease-activated receptors 1 and 2 (PAR-1 and 2, H. G. Selnick et al. Curr. Med. Chem. Cardiovasc. Hematol. Agents 2003, 1, 47-59; V. S. Ossovskaya et al. Physiol. Rev. 2004, 84, 579-621; A. M. Coelho et al. Curr. Med. Chem. Cardiovasc. Hematol. Agents 2003, 1, 61-72; M. Steinhoff et al. Endocrin. Rev. 2005, 26, 1-43).

Some of the transcription factor/transcriptional coactivator interactions that are of therapeutic relevance are:

HIF-1α/p300 (A. L. Kung, S. D. Zabludoff, D. S. France et al. Cancer Cell 2004, 6, 33), Tcf4/β-catenin (M. Lepourcelet, Y. N. P. Chen, D. S. France et al. Cancer Cell 2004, 5, 91), ERα/SRC-2, ERβ/SRC-2, TRβ/SRC-2 (T. R. Geistlinger, R. K. Guy, J. Am. Chem. Soc. 2003, 125, 6852), ESX/Sur2 (H. Shimogawa, Y. Kwon, Q. Mao et al. J. Am. Chem. Soc. 2004, 126, 3461).

In the compounds described below, a new strategy is introduced to stabilize β-hairpin conformations in backbone-turn peptidomimetics exhibiting selective agonizing or antagonizing activity against urotensin II, or inhibition of the STAT6/NCoA-1 interaction. This involves transplanting the hairpin sequence onto a template, whose function is to restrain the peptide loop backbone into hairpin geometry.

Template-bound hairpin mimetic peptides have been described in the literature (D. Obrecht, M. Altorfer, J. A. Robinson, Adv. Med. Chem. 1999, 4, 1-68; J. A. Robinson, Syn. Lett. 2000, 4, 429-441), but such molecules have not previously been evaluated or disclosed for development of agonizing or antagonizing activity against urotensin II, or inhibition of the STAT6/NCoA-1 interaction. However, the ability to generate β-hairpin peptidomimetics using combinatorial and parallel synthesis methods has now been established (L. Jiang, K. Moehle, B. Dhanapal, D. Obrecht, J. A. Robinson, Helv. Chim. Acta 2000, 83, 3097-3112). These methods allow the synthesis and screening of large hairpin mimetic libraries, which in turn considerably facilitates structure-activity studies, and hence the discovery of new molecules with potent selective agonizing or antagonizing activity.

β-Hairpin peptidomimetics obtained by the approach described here are useful for treating renal disease, diabetes, cardiovascular dysfunction, inflammation as well as allergic airways diseases like allergic rhinitis and asthma.

The β-hairpin peptidomimetics of the present invention are compounds of the general formula

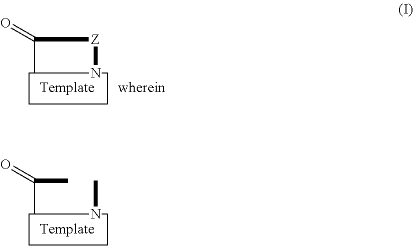

is a group of one of the formulae

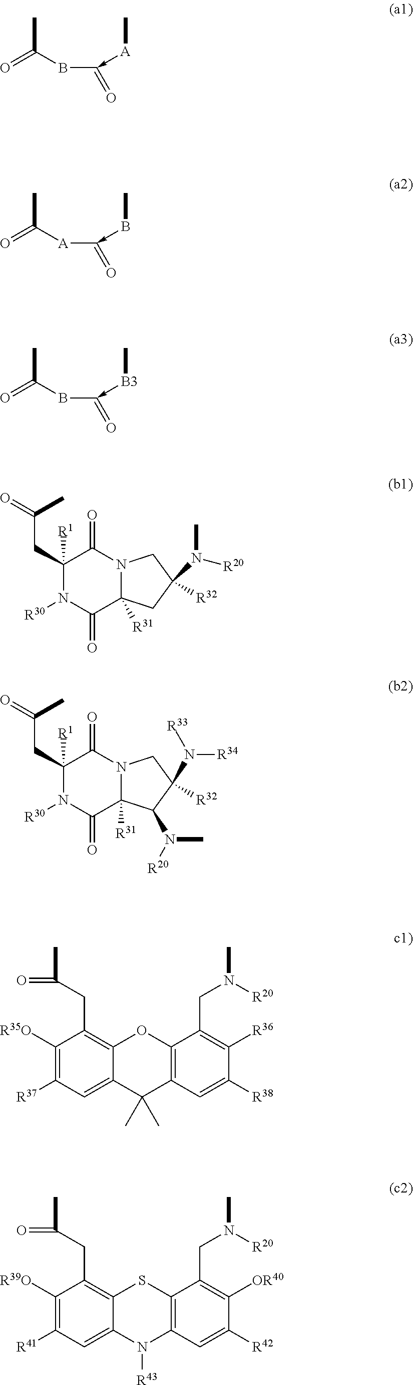

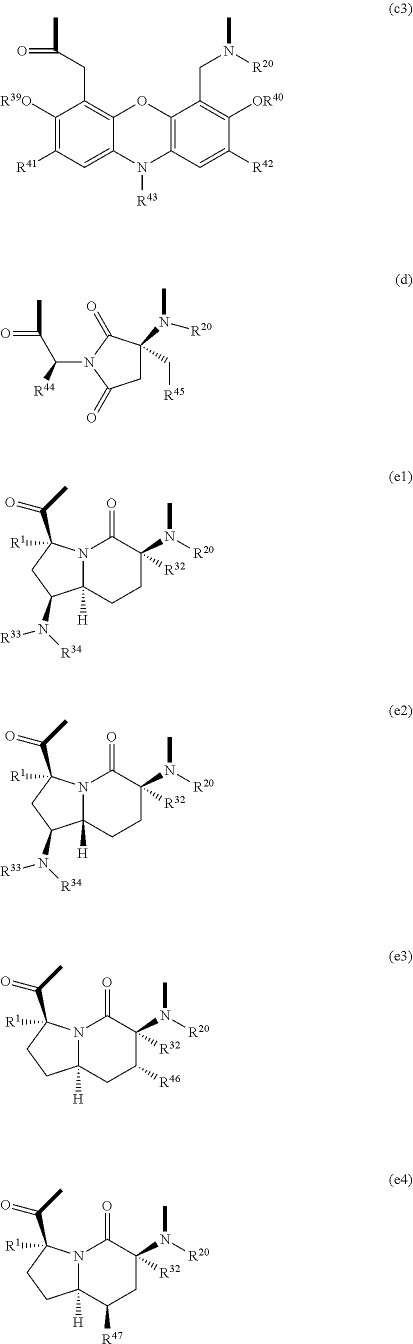

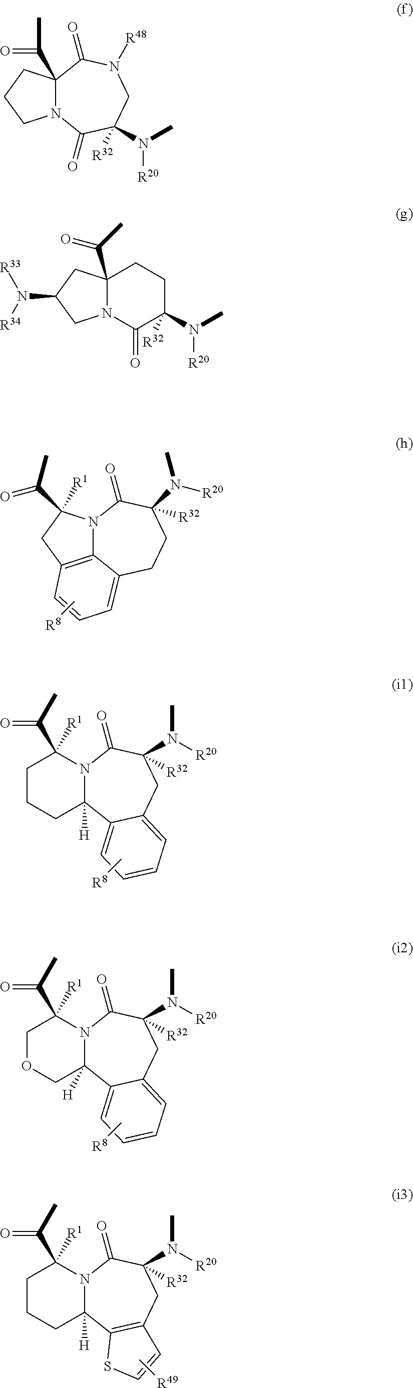

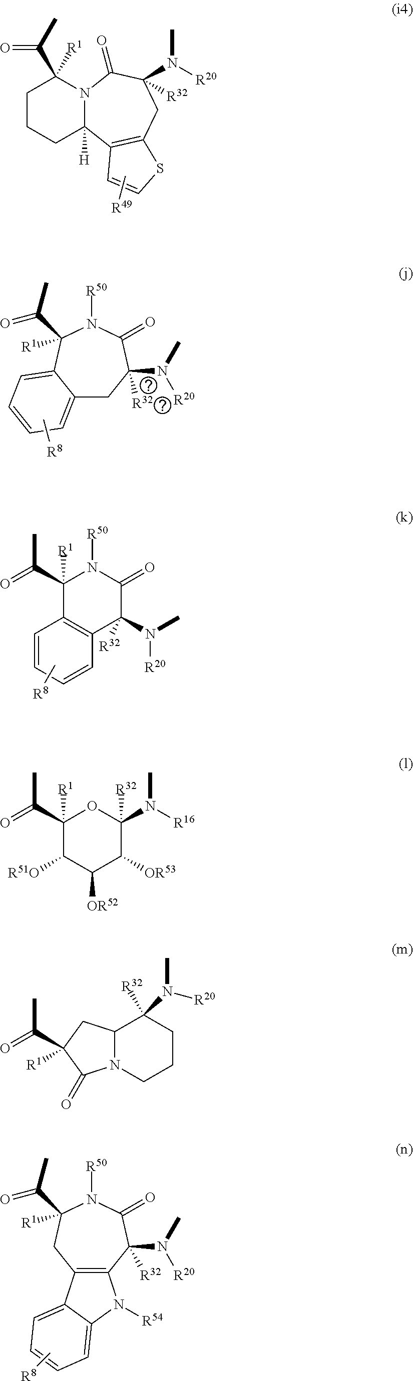

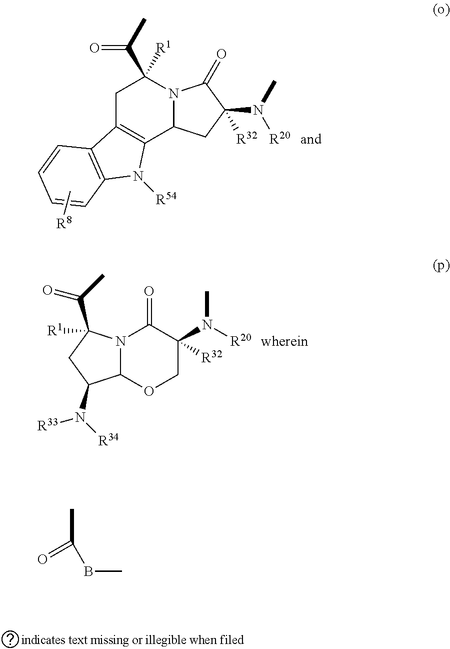

is Gly or the residue of an L-α-amino acid with B being a residue of formula —NR²⁰CH(R⁷¹)— or the enantiomer of one of the groups A1 to A69 and A105 as defined hereinafter;

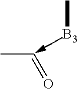

is Gly or the residue of a D-α-amino acid with B3 being a residue of formula —NR²⁰CH(R⁷¹)—;

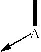

is a group of one of the formulae

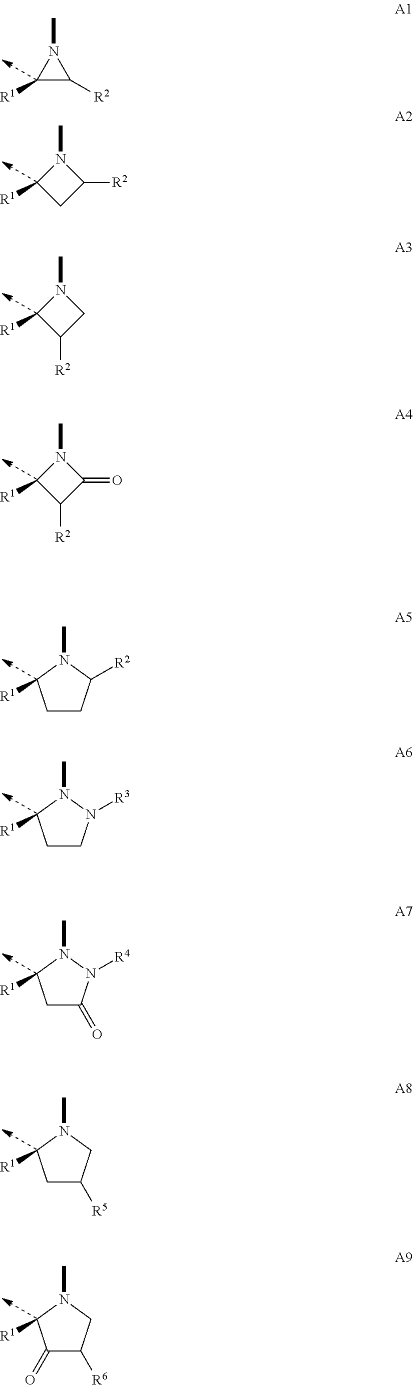

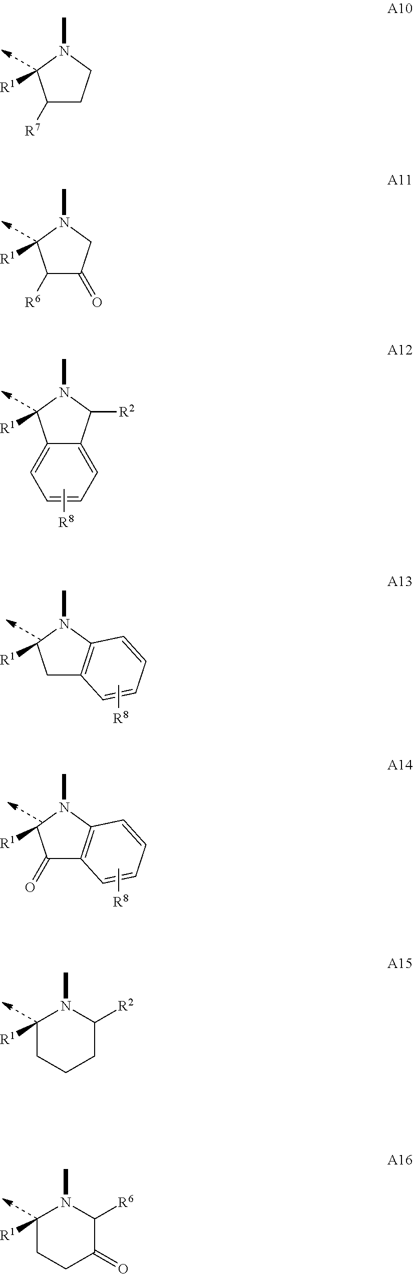

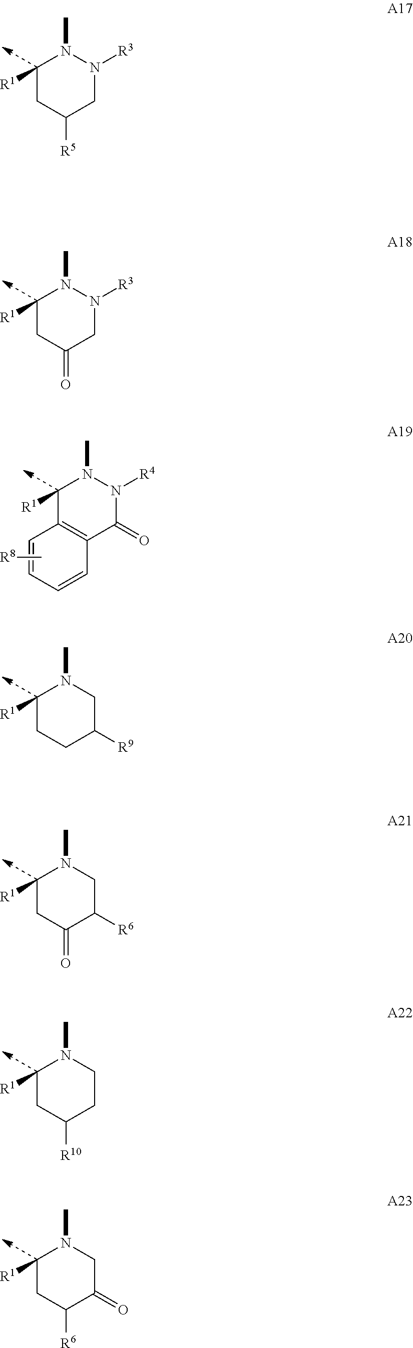

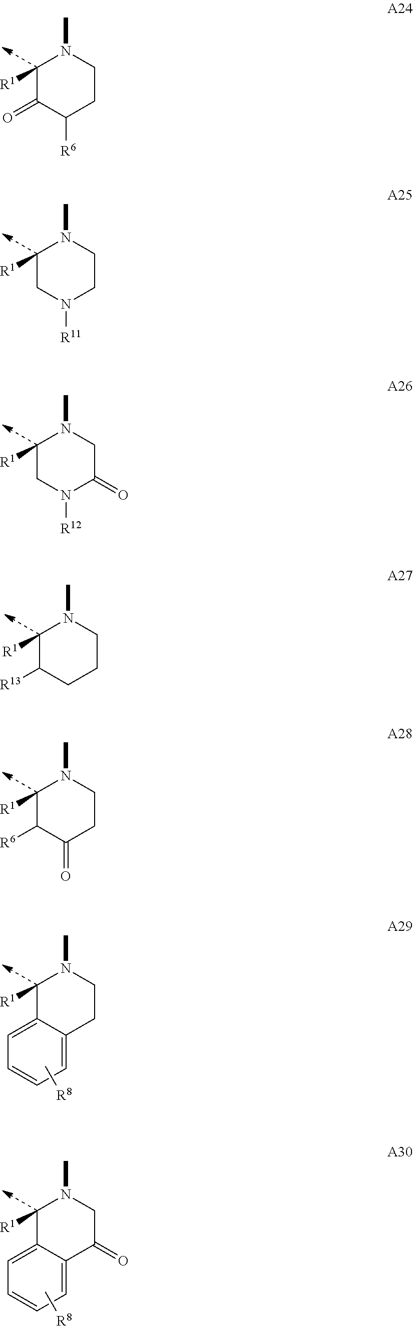

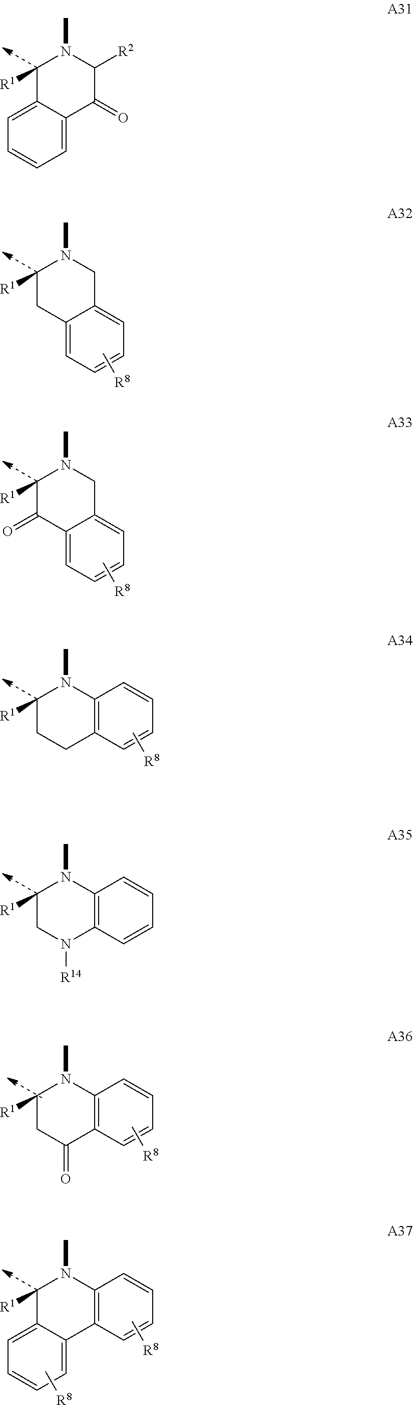

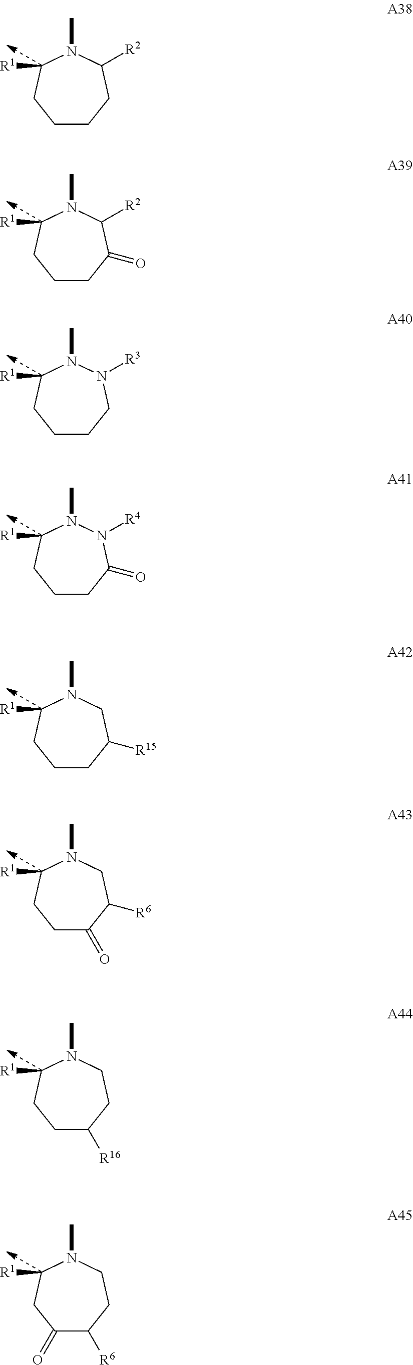

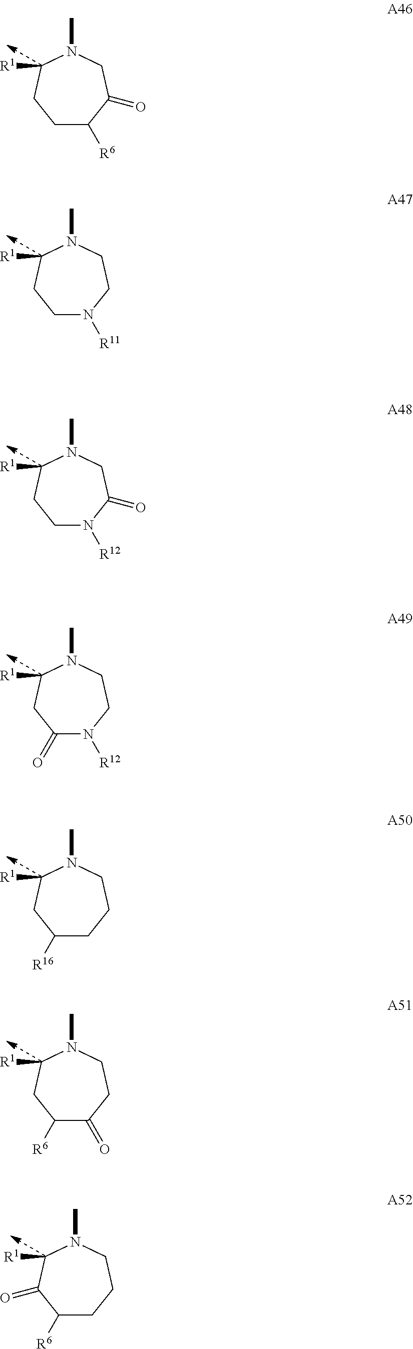

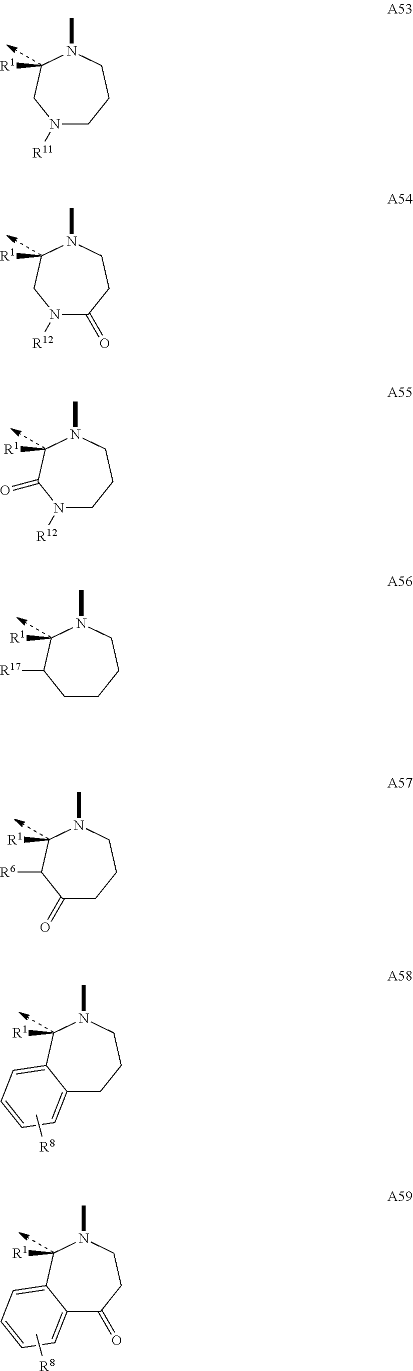

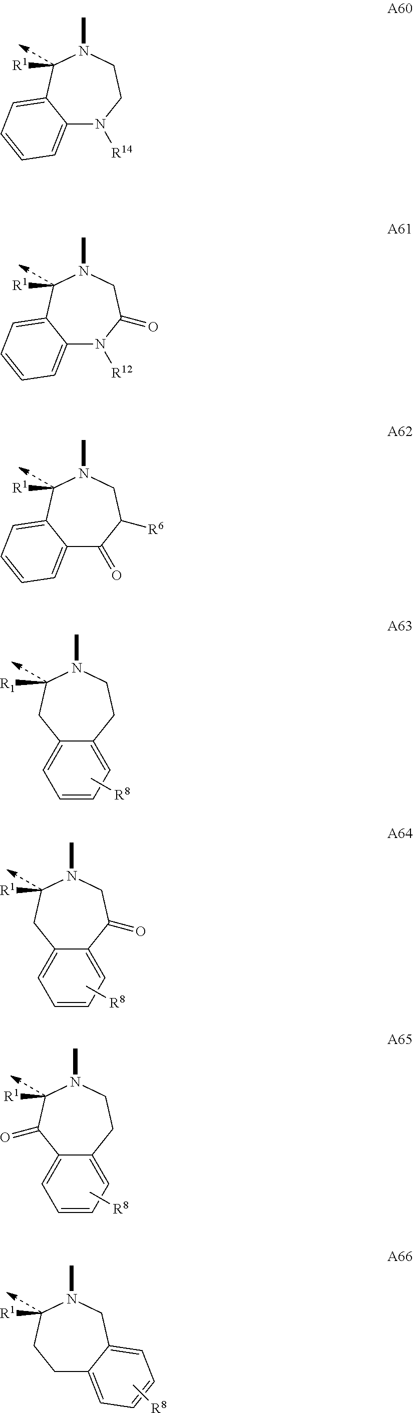

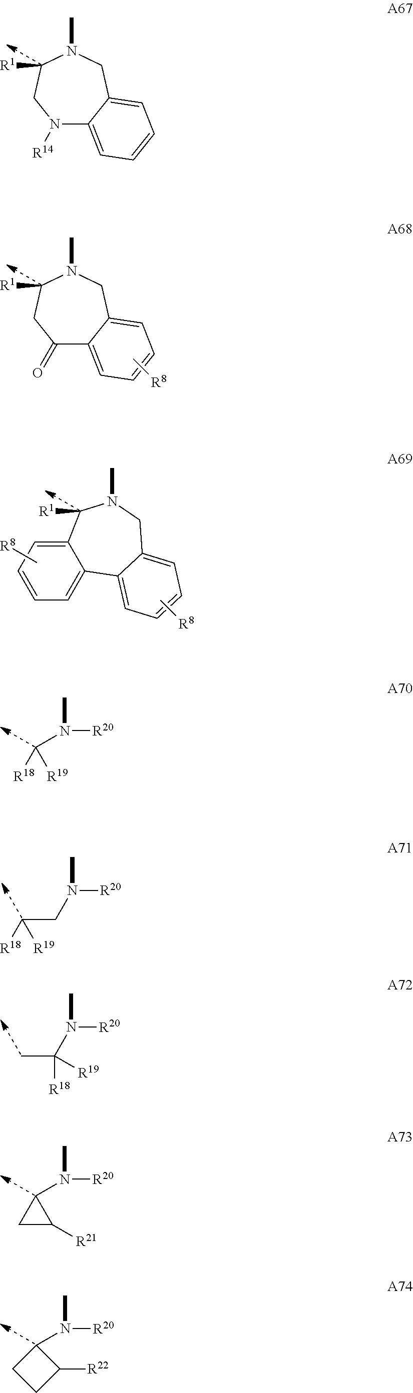

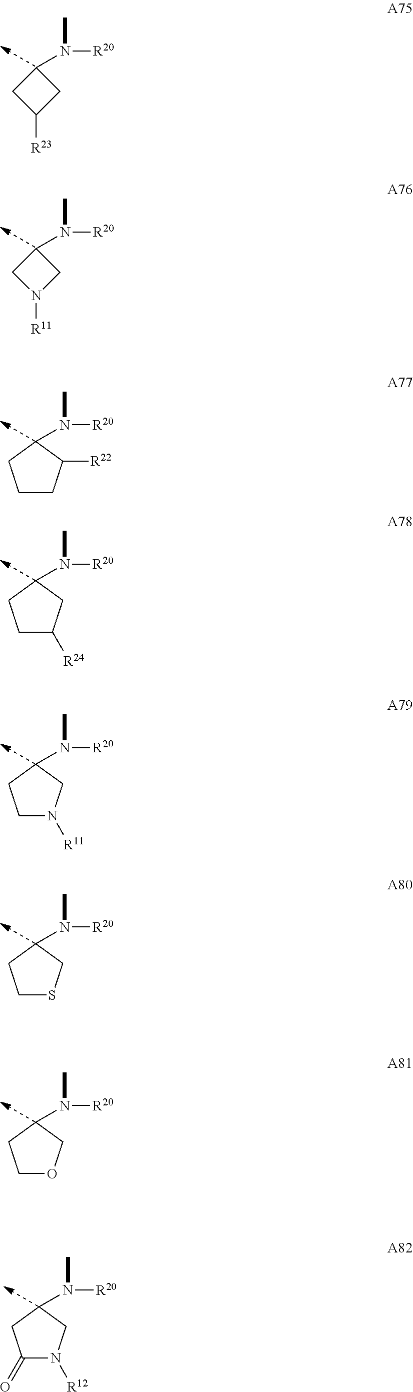

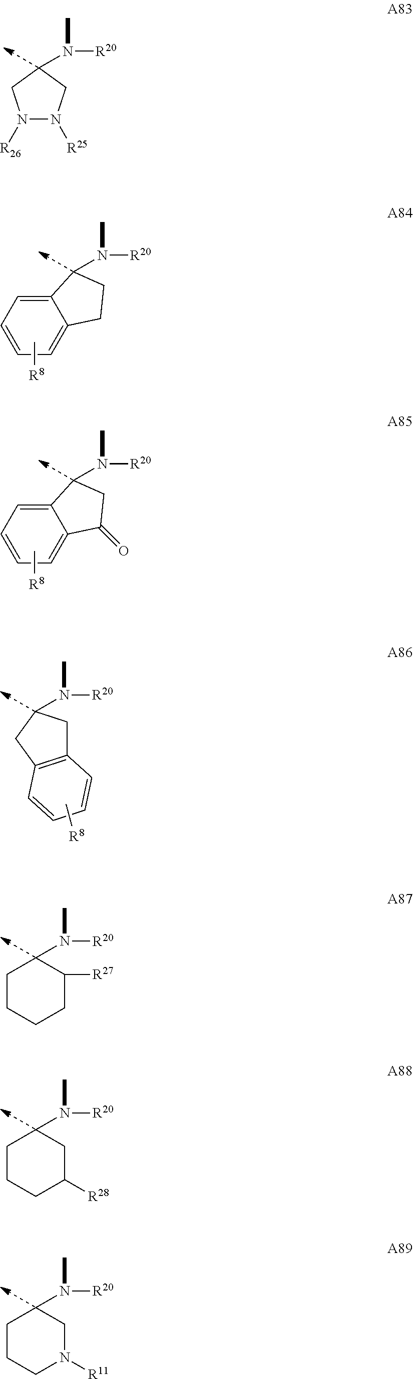

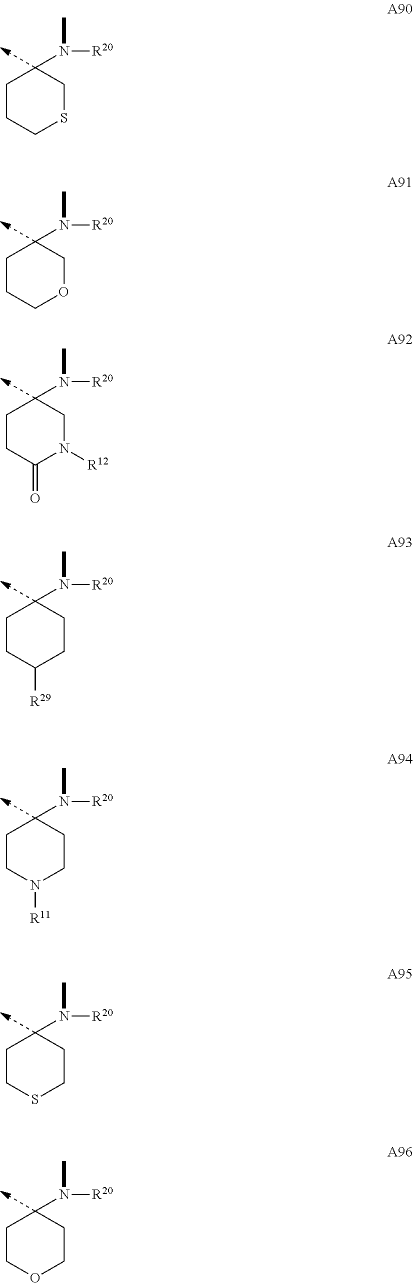

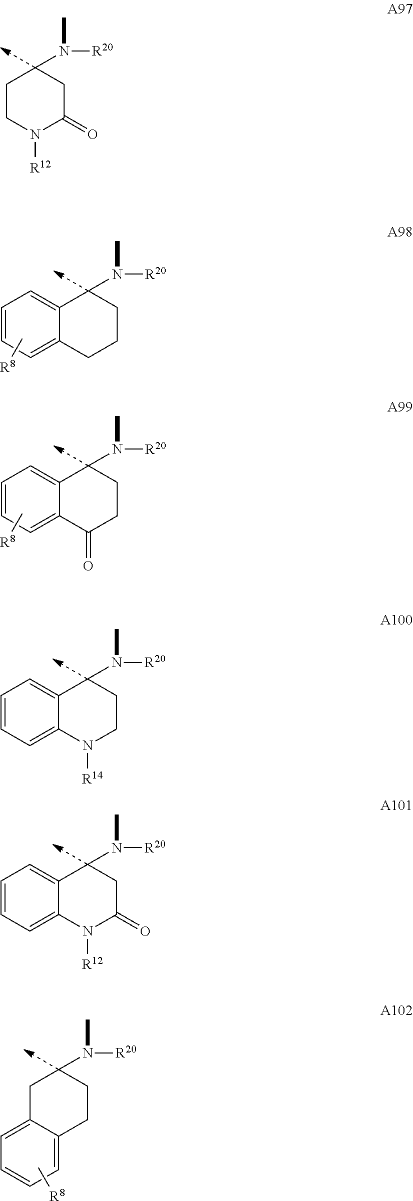

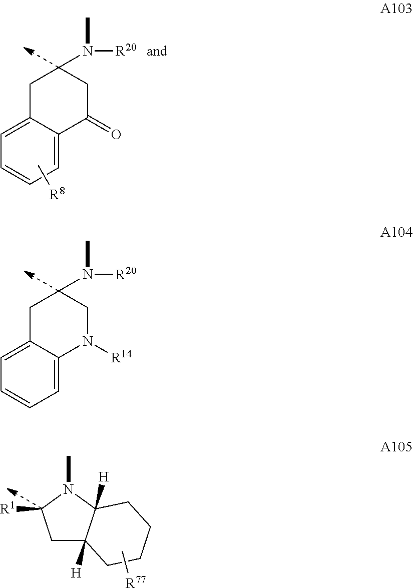

R¹ is H; lower alkyl; or aryl-lower alkyl;
R² is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₛPO(OR⁶⁰)₂;
   —(CH₂)ₒ(CHR⁶¹)ₒSO₂R⁶²; or —(CH₂)ₒ(CHR⁶¹)ₛR⁷⁷;
R³ is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R⁴ is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴;
   —(CH₂)ₘ(CHR⁶¹)ₛOCONR³³R⁷⁵; —(CH₂)ₘ(CHR⁶¹)ₛNR²⁰CONR³³R⁸²;
   —(CH₂)ₚ(CHR⁶¹)ₛCOOR⁵⁷; —(CH₂)ₚ(CHR⁶¹)ₛCONR⁵⁸R⁵⁹; —(CH₂)ₚ(CHR⁶¹)ₛPO(OR⁶⁰)₂;
   —(CH₂)ₚ(CHR⁶¹)ₛSO₂R⁶²; or —(CH₂)ₒ(CHR⁶¹)ₛC₆H₄R^{S};
R⁵ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R⁶ is H; alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R⁷ is alkyl; alkenyl; —(CH₂)ₒ(CHR⁶¹)ₛOR⁵⁵; —(CH₂)_{q}(CHR⁶¹)ₛNR³³R³⁴;
   —(CH₂)_{q}(CHR⁶¹)ₛOCONR³³R⁷⁵; —(CH₂)_{q}(CHR⁶¹)ₛNR²⁰CONR³³R⁸²;
   —(CH₂)ᵣ(CHR⁶¹)ₛCOOR⁵⁷; —(CH₂)ᵣ(CHR⁶¹)ₛCONR⁵⁸R⁵⁹;
   —(CH₂)ᵣ(CHR⁶¹)ₛPO(OR⁶⁰)₂;
   —(CH₂)ᵣ(CHR⁶¹)ₛSO₂R⁶²; or —(CH₂)ᵣ(CHR⁶¹)ₛC₆H₄R^{S};
R^{S} is H; Cl; F; CF₃; NO₂; lower alkyl; lower alkenyl; aryl; aryl-lower alkyl; —(CH₂)ₒ(CHR⁶¹)ₛOR⁵⁵; —(CH₂)ₒ(CHR⁶¹)ₛSR⁵⁶; —(CH₂)ₒ(CHR⁶¹)NR³³R³⁴; —(CH₂)ₒ(CHR⁶¹)ₛOCONR³³R⁷⁵; —(CH₂)ₒ(CHR⁶¹)ₛNR²⁰CONR³³R⁸²; —(CH₂)ₒ(CHR⁶¹)ₛCOOR⁵⁷; —(CH₂)ₒ(CHR⁶¹)ₛCONR⁵⁸R⁵⁹; —(CH₂)ₒ(CHR⁶¹)ₛPO(OR⁶⁰)₂; —(CH₂)ₒ(CHR⁶¹)ₛSO₂R⁶²; —(CH₂)ₒ(CHR⁶¹)ₛCOR⁶⁴;
R⁹ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R¹⁰ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R¹¹ is H; alkyl; alkenyl; —(CH₂)ₘ(CHR⁶¹)ₛOR⁵⁵; —(CH₂)ₘ(CHR⁶¹)ₛNR³³R³⁴;
   —(CH₂)ₘ(CHR⁶¹)ₛOCONR³³R⁷⁵; —(CH₂)ₘ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R¹² is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ᵣ(CHR⁶¹)ₛCOOR⁵⁷;
   —(CH₂)ᵣ(CHR⁶¹)ₛCONR⁵⁸R⁵⁹; —(CH₂)ᵣ(CHR⁶¹)ₛPO(OR⁶⁰)₂; —(CH₂)ᵣ(CHR⁶¹)ₛSO₂R⁶²;
   or —(CH₂)ᵣ(CHR⁶¹)ₛC₆H₄R^{S};
R¹³ is alkyl; alkenyl; —(CH₂)_{q}(CHR⁶¹)ₛOR⁵⁵; —(CH₂)_{q}(CHR⁶¹)ₛSR⁵⁶;
   —(CH₂)_{q}(CHR⁶¹)ₛNR³³R³⁴;
   —(CH₂)_{q}(CHR⁶¹)ₛOCONR³³R⁷⁵; —(CH₂)_{q}(CHR⁶¹)ₛNR²⁰CONR³³R⁸²;
   —(CH₂)_{q}(CHR⁶¹)ₛCOOR⁵⁷; —(CH₂)_{q}(CHR⁶¹)ₛCONR⁵⁸R⁵⁹;
   —(CH₂)_{q}(CHR⁶¹)ₛPO(OR⁶⁰)₂;
   —(CH₂)_{q}(CHR⁶¹)ₛSO₂R⁶²; or —(CH₂)_{q}(CHR⁶¹)ₛC₆H₄R^{S};
R¹⁴ is H; alkyl; alkenyl; —(CH₂)ₘ(CHR⁶¹)ₛNR³³R³⁴;
   —(CH₂)ₘ(CHR⁶¹)ₛOCONR³³R⁷⁵; —(CH₂)ₘ(CHR⁶¹)ₛNR²⁰CONR³³R⁸²;
   —(CH₂)ₘ(CHR⁶¹)ₛCOOR⁵⁷; —(CH₂)_{q}(CHR⁶¹)ₛCONR⁵⁸R⁵⁹;
   —(CH₂)_{q}(CHR⁶¹)ₛPO(OR⁶⁰)₂;
   —(CH₂)_{q}(CHR⁶¹)ₛSOR⁶²; or —(CH₂)_{q}(CHR⁶¹)ₛC₆H₄R^{S};
R¹⁵ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R¹⁶ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R¹⁷ is alkyl; alkenyl; —(CH₂)_{q}(CHR⁶¹)ₛOR⁵⁵; —(CH₂)_{q}(CHR⁶¹)ₛSR⁵⁶; —
   (CH₂)_{q}(CHR⁶¹)ₛNR³³R³⁴;
   —(CH₂)_{q}(CHR⁶¹)ₛOCONR³³R⁷⁵; —(CH₂)_{q}(CHR⁶¹)ₛNR²⁰CONR³³R⁸²;
   —(CH₂)_{q}(CHR⁶¹)ₛCOOR⁵⁷; —(CH₂)_{q}(CHR⁶¹)ₛCONR⁵⁸R⁵⁹;
   —(CH₂)_{q}(CHR⁶¹)ₛPO(OR⁶⁰)₂;
   —(CH₂)_{q}(CHR⁶¹)ₛSO₂R⁶²; or —(CH₂)_{q}(CHR⁶¹)ₛC₆H₄R^{S};
R¹⁸ is alkyl; alkenyl; —(CH₂)ₚ(CHR⁶¹)ₛOR⁵⁵; —(CH₂)ₚ(CHR⁶¹)ₛSR⁵⁶; —(CH₂)ₚ(CHR⁶¹)ₛNR³³R³⁴;
   (CH₂)ₚ(CHR⁶¹)ₛOCONR³³R⁷⁵; —(CH₂)ₚ(CHR⁶¹)ₛNR²⁰CONR³³R⁸²;
   —(CH₂)ₚ(CHR⁶¹)ₛCOOR⁵⁷; —(CH₂)ₚ(CHR⁶¹)ₛCONR⁵⁸R⁵⁹;
   —(CH₂)ₚ(CHR⁶¹)ₛPO(OR⁶⁰)₂;
   —(CH₂)ₚ(CHR⁶¹)ₛSO₂R⁶²; or —(CH₂)ₒ(CHR⁶¹)ₛC₆H₄R^{S};
R¹⁹ is lower alkyl; —(CH₂)ₚ(CHR⁶¹)ₛOR⁵⁵; —(CH₂)ₚ(CHR⁶¹)ₛSR⁵⁶;
   —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₚ(CHR⁶¹)ₛCOOR⁵⁷; —(CH₂)ₚ(CHR⁶¹)ₛCONR⁵⁸R⁵⁹;
   —(CH₂)ₚ(CHR⁶¹)ₛPO(OR⁶⁰)₂;
   —(CH₂)ₚ(CHR⁶¹)ₛSO₂R⁶²; or —(CH₂)ₒ(CHR⁶¹)ₛC₆H₄R^{S}; or
R¹⁸ and R¹⁹ taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R²⁰ is H; alkyl; alkenyl; or aryl-lower alkyl;
R²¹ is H; alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R²² is H; alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R²³ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R²⁴ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₒSO₂R⁶²; or —(CH₂)ₒ(CHR⁶¹)ₛC₆H₄R^{S};
R²⁵ is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R²⁶ is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S}; or
R²⁵ and R²⁶ taken together can form: —(CH₂)₂₋₆—; —(CH₂)ᵣO(CH₂)ᵣ—; —(CH₂)ᵣS(CH₂)ᵣ—; or
   —(CH₂)ᵣNR⁵⁷(CH₂)ᵣ—;
R²⁷ is H; alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛOCONR³³R⁷⁵;
   —(CH₂)ₒ(CHR⁶¹)ₛNR²⁰CONR³³R⁸²; —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R²⁸ is alkyl; alkenyl; —(CH₂)ₒ(CHR⁶¹)ₛ—OR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹; —(CH₂)ₒ(CHR⁶¹)ₛPO(OR⁶⁰)₂;
   —(CH₂)ₒ(CHR⁶¹)ₒSO₂R⁶²; or —(CH₂)ₒ(CHR⁶¹)ₛC₆H₄R^{S};
R²⁹ is alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R³⁰ is H; alkyl; alkenyl; or aryl-lower alkyl;
R³¹ is H; alkyl; alkenyl; —(CH₂)ₚ(CHR⁶¹)ₛOR⁵⁵; —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R³² is H; lower alkyl; or aryl-lower alkyl;
R³³ is H; alkyl, alkenyl; —(CH₂)ₘ(CHR⁶¹)ₛOR⁵⁵; —(CH₂)ₘ(CHR⁶¹)ₛNR³⁴R⁶³;
   —(CH₂)ₘ(CHR⁶¹)ₛOCONR⁷⁵R⁸²; —(CH₂)ₘ(CHR⁶¹)ₛNR²⁰CONR⁷⁸R⁸²;
   —(CH₂)ₒ(CHR⁶¹)ₛCOR⁶⁴; —(CH₂)ₒ(CHR⁶¹)ₛ—CONR⁵⁸R⁵⁹,
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R³⁴ is H; lower alkyl; aryl, or aryl-lower alkyl; or
R³³ and R³⁴ taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R³⁵ is H; alkyl; alkenyl; —(CH₂)ₘ(CHR⁶¹)ₛOR⁵⁵; —(CH₂)ₘ(CHR⁶¹)ₛNR³³R³⁴;
   —(CH₂)ₘ(CHR⁶¹)ₛOCONR³³R⁷⁵; —(CH₂)ₘ(CHR⁶¹)ₛNR²⁰CONR³³R⁸²;
   —(CH₂)ₚ(CHR⁶¹)ₛCOOR⁵⁷; —(CH₂)ₚ(CHR⁶¹)ₛCONR⁵⁸R⁵⁹;
   —(CH₂)ₚ(CHR⁶¹)ₛPO(OR⁶⁰)₂;
   —(CH₂)ₚ(CHR⁶¹)ₛSO₂R⁶²; or —(CH₂)ₚ(CHR⁶¹)ₛC₆H₄R^{S};
R³⁶ is H, alkyl; alkenyl; —(CH₂)ₒ(CHR⁶¹)ₛOR⁵⁵; —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₚ(CHR⁶¹)ₛCOOR⁵⁷; —(CH₂)ₚ(CHR⁶¹)ₛCONR⁵⁸R⁵⁹;
   —(CH₂)ₚ(CHR⁶¹)ₛPO(OR⁶⁰)₂;
   —(CH₂)ₚ(CHR⁶¹)ₛSO₂R⁶²; or —(CH₂)ₒ(CHR⁶¹)ₛC₆H₄R^{S};
R³⁷ is H; F; Br; Cl; NO₂; CF₃; lower alkyl; —(CH₂)ₚ(CHR⁶¹)ₛOR⁵⁵;
   —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R³⁸ is H; F; Br; Cl; NO₂; CF₃; alkyl; alkenyl; —(CH₂)ₚ(CHR⁶¹)ₛOR⁵⁵;
   —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R³⁹ is H; alkyl; alkenyl; or aryl-lower alkyl;
R⁴⁰ is H; alkyl; alkenyl; or aryl-lower alkyl;
R⁴¹ is H; F; Br; Cl; NO₂; CF₃; alkyl; alkenyl; —(CH₂)ₚ(CHR⁶¹)ₛOR⁵⁵;
   —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R⁴² is H; F; Br; Cl; NO₂; CF₃; alkyl; alkenyl; —(CH₂)ₚ(CHR⁶¹)ₛOR⁵⁵;
   —(CH₂)ₚ(CHR⁶¹)ₛNR³³R³⁴;
   —(CH₂)ₚ(CHR⁶¹)ₛOCONR³³R⁷⁵; —(CH₂)ₚ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R⁴³ is H; alkyl; alkenyl; —(CH₂)ₘ(CHR⁶¹)ₛOR⁵⁵; —(CH₂)ₘ(CHR⁶¹)ₛNR³³R³⁴;
   —(CH₂)ₘ(CHR⁶¹)ₛOCONR³³R⁷⁵; —(CH₂)ₘ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₒ(CHR⁶¹)ₛPO(OR⁶⁰)₂;
   —(CH₂)ₒ(CHR⁶¹)ₛSO₂R⁶²; or —(CH₂)ₒ(CHR⁶¹)ₛC₆H₄R^{S};
R⁴⁴ is alkyl; alkenyl; —(CH₂)ᵣ(CHR⁶¹)ₛOR⁵⁵; —(CH₂)ᵣ(CHR⁶¹)ₛSR⁵⁶;
   —(CH₂)ᵣ(CHR⁶¹)ₛNR³³R³⁴;
   —(CH₂)ᵣ(CHR⁶¹)ₛOCONR³³R⁷⁵; —(CH₂)ᵣ(CHR⁶¹)ₛNR²⁰CONR³³R⁸²;
   —(CH₂)ᵣ(CHR⁶¹)ₛCOOR⁵⁷; —(CH₂)ᵣ(CHR⁶¹)ₛCONR⁵⁸R⁵⁹;
   —(CH₂)ᵣ(CHR⁶¹)ₛPO(OR⁶⁰)₂;
   —(CH₂)ᵣ(CHR⁶¹)ₒSO₂R⁶²; or —(CH₂)ᵣ(CHR⁶¹)ₛC₆H₄R^{S};
R⁴⁵ is H; alkyl; alkenyl; —(CH₂)ₒ(CHR⁶¹)ₛOR⁵⁵; —(CH₂)ₒ(CHR⁶¹)ₛSR⁵⁶;
   —(CH₂)ₒ(CHR⁶¹)ₛNR³³R³⁴;
   —(CH₂)ₒ(CHR⁶¹)ₛOCONR³³R⁷⁵; —(CH₂)ₒ(CHR⁶¹)ₛNR²⁰CONR³³R⁸²;
   —(CH₂)ₒ(CHR⁶¹)ₛCOOR⁵⁷; —(CH₂)ₒ(CHR⁶¹)ₛCONR⁵⁸R⁵⁹;
   —(CH₂)ₛ(CHR⁶¹)ₛPO(OR⁶⁰)₂;
   —(CH₂)ₛ(CHR⁶¹)ₒSO₂R⁶²; or —(CH₂)ₛ(CHR⁶¹)ₛC₆H₄R^{S};
R⁴⁶ is H; alkyl; alkenyl; or —(CH₂)ₒ(CHR⁶¹)ₚC₆H₄R^{S};
R⁴⁷ is H; alkyl; alkenyl; or —(CH₂)ₒ(CHR⁶¹)ₛOR⁵⁵;
R⁴⁸ is H; lower alkyl; lower alkenyl; or aryl-lower alkyl;
R⁴⁹ is H; alkyl; alkenyl; —(CHR⁶¹)ₛCOOR⁵⁷; (CHR⁶¹)ₛCONR⁵⁸R⁵⁹; (CHR⁶¹)ₛPO(OR⁶⁰)₂;
   —(CHR⁶¹)ₛSOR⁶²; or —(CHR⁶¹)ₛC₆H₄R^{S};
R⁵⁰ is H; lower alkyl; or aryl-lower alkyl;
R⁵¹ is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₚPO(OR⁶⁰)₂;
   —(CH₂)ₚ(CHR⁶¹)ₛSO₂R⁶²; or —(CH₂)ₚ(CHR⁶¹)ₛC₆H₄R^{S};
R⁵² is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR³³R⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₚPO(OR⁶⁰)₂;
   —(CH₂)ₚ(CHR⁶¹)ₛSO₂R⁶²; or —(CH₂)ₚ(CHR⁶¹)ₛC₆H₄R^{S};
R⁵³ is H; alkyl; alkenyl; —(CH₂)ₘ(CHR⁶¹)ₛOR⁵⁵; —(CH₂)ₘ(CHR⁶¹)ₛSR⁵⁶;
   —(CH₂)ₘ(CHR⁶¹)ₛNR³³R³⁴; —(CH₂)ₘ(CHR⁶¹)ₛOCONR⁷⁵;
   —(CH₂)ₘ(CHR⁶¹)ₛNR²⁰CONR³³R⁸²; —(CH₂)ₒ(CHR⁶¹)ₛCOOR⁵⁷;
   —(CH₂)ₒ(CHR⁶¹)ₛCONR⁵⁸R⁵⁹; —(CH₂)ₒ(CHR⁶¹)ₛPO(OR⁶⁰)₂;
   —(CH₂)ₚ(CHR⁶¹)ₛSO₂R⁶²; or —(CH₂)ₚ(CHR⁶¹)ₛC₆H₄R^{S};
R⁵⁴ is H; alkyl; alkenyl; —(CH₂)ₘ(CHR⁶¹)ₛOR⁵⁵; —(CH₂)ₘ(CHR⁶¹)ₛNR³³R³⁴;
   —(CH₂)ₘ(CHR⁶¹)ₛOCONR³³R⁷⁵; —(CH₂)ₘ(CHR⁶¹)ₛNR²⁰CONR³³R⁸²;
   —(CH₂)ₒ(CHR⁶¹)COOR⁵⁷; —(CH₂)ₒ(CHR⁶¹)ₛCONR⁵⁸R⁵⁹; or
   —(CH₂)ₒ(CHR⁶¹)ₛC₆H₄R^{S};
R⁵⁵ is H; lower alkyl; lower alkenyl; aryl-lower alkyl; —(CH₂)ₘ(CHR⁶¹)ₛOR⁵⁷;
   —(CH₂)ₘ(CHR⁶¹)ₛNR³⁴R⁶³; —(CH₂)ₘ(CHR⁶¹)ₛOCONR⁷⁵R⁸²;
   —(CH₂)ₘ(CHR⁶¹)ₛNR²⁰CONR⁷⁸R⁸²; —(CH₂)ₒ(CHR⁶¹)ₛ—COR⁶⁴;
   —(CH₂)ₒ(CHR⁶¹)COOR⁵⁷; or
   —(CH₂)ₒ(CHR⁶¹)ₛCONR⁵⁸R⁵⁹;
R⁵⁶ is H; lower alkyl; lower alkenyl; aryl-lower alkyl; —(CH₂)ₘ(CHR⁶¹)ₛOR⁵⁷;
   —(CH₂)ₘ(CHR⁶¹)ₛNR³⁴R⁶³; —(CH₂)ₘ(CHR⁶¹)ₛOCONR⁷⁵R⁸²;
   —(CH₂)ₘ(CHR⁶¹)ₛNR²⁰CONR⁷⁸R⁸²; —(CH₂)ₒ(CHR⁶¹)ₛ—COR⁶⁴; or
   —(CH₂)ₒ(CHR⁶¹)ₛCONR⁵⁸R⁵⁹;
R⁵⁷ is H; lower alkyl; lower alkenyl; aryl lower alkyl; or heteroaryl lower alkyl;
R⁵⁸ is H; lower alkyl; lower alkenyl; aryl; heteroaryl; aryl-lower alkyl; or heteroaryl-lower alkyl;
R⁵⁹ is H; lower alkyl; lower alkenyl; aryl; heteroaryl; aryl-lower alkyl; or heteroaryl-lower alkyl; or
R⁵⁸ and R⁵⁹ taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R⁶⁰ is H; lower alkyl; lower alkenyl; aryl; or aryl-lower alkyl;
R⁶¹ is alkyl; alkenyl; aryl; heteroaryl; aryl-lower alkyl; heteroaryl-lower alkyl;
   —(CH₂)ₘOR⁵⁵;
   —(CH₂)ₘNR³³R³⁴; —(CH₂)ₘOCONR⁷⁵R⁸²; —(CH₂)ₘNR²⁰CONR⁷⁸R⁸²;
   —(CH₂)ₒCOOR³⁷;
   —(CH₂)ₒNR⁵⁸R⁵⁹; or —(CH₂)ₒPO(COR⁶⁰)₂;
R⁶² is lower alkyl; lower alkenyl; aryl, heteroaryl; or aryl-lower alkyl;
R⁶³ is H; lower alkyl; lower alkenyl; aryl, heteroaryl; aryl-lower alkyl; heteroaryl-lower alkyl;
   —COR⁶⁴; —COOR⁵⁷; —CONR⁵⁸R⁵⁹; —SO₂R⁶²; or —PO(OR⁶⁰)₂; or
R³⁴ and R⁶³ taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R⁶⁴ is H; lower alkyl; lower alkenyl; aryl; heteroaryl; aryl-lower alkyl; heteroaryl-lower alkyl;
   —(CH₂)ₚ(CHR⁶¹)ₛOR⁶⁵; —(CH₂)ₚ(CHR⁶¹)ₛSR⁶⁶; or —(CH₂)ₚ(CHR⁶¹)ₛNR³⁴R⁶³;
   —(CH₂)ₚ(CHR⁶¹)ₛOCONR⁷⁵R⁸²; —(CH₂)_{P}(CHR⁶¹)ₛNR²⁰CONR⁷⁸R⁸²;
R⁶⁵ is H; lower alkyl; lower alkenyl; aryl, aryl-lower alkyl; heteroaryl-lower alkyl;
   —COR⁵⁷;
   —COOR⁵⁷; or —CONR⁵⁸R⁵⁹;
R⁶⁶ is H; lower alkyl; lower alkenyl; aryl; aryl-lower alkyl; heteroaryl-lower alkyl; or
   —CONR⁵⁸R⁵⁹;
R⁶⁷ is H; Cl; Br; F; NO₂; —NR³⁴COR⁵⁷; lower alkyl; or lower alkenyl;
R⁶⁸ is H; Cl; Br; F; NO₂; —NR³⁴COR⁵⁷; lower alkyl; or lower alkenyl;
R⁶⁹ is H; Cl; Br; F; NO₂; —NR³⁴COR⁵⁷; lower alkyl; or lower alkenyl;
R⁷⁰ is H; Cl; Br; F; NO₂; —NR³⁴COR⁵⁷; lower alkyl; or lower alkenyl;
with the proviso that at least two of R⁶⁷, R⁶⁸, R⁶⁹ and R⁷⁰ are H;
R⁷¹ is lower alkyl; lower alkenyl; —(CH₂)ₚ(CHR⁶¹)ₛOR⁷⁵; —(CH₂)ₚ(CHR⁶¹)ₛSR⁷⁵;
   —(CH₂)ₚ(CHR⁶¹)ₛNR³³R³⁴; —(CH₂)ₚ(CHR⁶¹)ₛOCONR³³R⁷⁵;
   —(CH₂)ₚ(CHR⁶¹)ₛNR²⁰CONR³³R⁸²;
   —(CH₂)ₒ(CHR⁶¹)ₛCOOR⁷⁵; —(CH₂)ₚCONR⁵⁸R⁵⁹; —(CH₂)ₚPO(OR⁶²)₂; —(CH₂)ₚSO₂R⁶²; or
   —(CH₂)ₒ—C₆R⁶⁷R⁶⁸R⁶⁹R⁷⁰R⁷⁶;

Z is a chain of 8 α-amino acid residues, the positions of said amino acid residues in said chain being counted starting from the N-terminal amino acid, whereby these amino acid residues are, depending on their position in the chains, Gly, Pro or of one of the types

### C: —NR²⁰CH(R⁷²)CO—;

### D: —NR²⁰CH(R⁷³)CO—;

### E: —NR²⁰CH(R⁷⁴)CO—;

### F: —NR²⁰CH(R⁸⁴)CO—; and

### H: —NR²⁰—CH(CO—)—(CH₂)₄₋₇—CH(CO—)—NR²⁰—;

—NR²⁰—CH(CO—)—(CH₂)ₚSS(CH₂)ₚ—CH(CO—)—NR²⁰—;
—NR²⁰—CH(CO—)—(—(CH₂)ₚNR²⁰CO(CH₂)ₚ—CH(CO—)—NR²⁰—; or
—NR²⁰—CH(CO—)—(—(CH₂)ₚNR²⁰CONR²⁰(CH₂)ₚ—CH(CO—)—NR²⁰—;
R⁷² is H, lower alkyl; lower alkenyl; —(CH₂)ₚ(CHR⁶¹)ₛOR⁸⁵; or —(CH₂)ₚ(CHR⁶¹)ₛSR⁸⁵;
R⁷³ is —(CH₂)ₒR⁷⁷; —(CH₂)ᵣO(CH₂)ₒR⁷⁷; —(CH₂)ᵣS(CH₂)ₒR⁷⁷; or —(CH₂)ᵣNR²⁰(CH₂)ₒR⁷⁷;
R⁷⁴ is —(CH₂)ₚNR⁷⁸R⁷⁹; —(CH₂)ₚNR⁷⁷R⁸⁰; —(CH₂)ₚC(═NR⁸⁰)NR⁷⁸R⁷⁹;
   —(CH₂)ₚC(═NOR⁵⁰)NR⁷⁸R⁷⁹;
   —(CH₂)ₚC(═NNR⁷⁸R⁷⁹)NR⁷⁸R⁷⁹; —(CH₂)ₚNR⁸⁰C(═NR⁸⁰)NR⁷⁸R⁷⁹;
   —(CH₂)ₚN═C(NR⁷⁸R⁸⁰)NR⁷⁹R⁸⁰; —(CH₂)ₚC₆H₄NR⁷⁸R⁷⁹; —(CH₂)ₚC₆H₄NR⁷⁷R⁸⁰;
   —(CH₂)ₚC₆H₄C(═NR⁸⁰)NR⁷⁸R⁷⁹; —(CH₂)ₚC₆H₄C(═NOR⁵⁰)NR⁷⁸R⁷⁹;
   —(CH₂)ₚC₆H₄C(═NNR⁷⁸R⁷⁹)NR⁷⁸R⁷⁹; —(CH₂)ₚC₆H₄NR⁸⁰C(═NR⁸⁰)NR⁷⁸R⁷⁹;
   —(CH₂)ₚC₆H₄N═C(NR⁷⁸R⁸⁰)NR⁷⁹R⁸⁰; —(CH₂)ᵣO(CH₂)ₘNR⁷⁸R⁷⁹;
   —(CH₂)ᵣO(CH₂)ₘNR⁷⁷R⁸⁰;
   —(CH₂)ᵣO(CH₂)ₚC(═NR⁸⁰)NR⁷⁸R⁷⁹; —(CH₂)ᵣO(CH₂)ₚC(═NOR⁵⁰)NR⁷⁸R⁷⁹;
   —(CH₂)ᵣO(CH₂)ₚC(═NNR⁷⁸R⁷⁹)NR⁷⁸R⁷⁹; —(CH₂)ᵣO(CH₂)ₘNR⁸⁰C(═NR⁸⁰)NR⁷⁸R⁷⁹;
   —(CH₂)ᵣO(CH₂)ₘN═C(NR⁷⁸R⁸⁰)NR⁷⁹R⁸⁰; —(CH₂)ᵣO(CH₂)ₚC₆H₄CNR⁷⁸R⁷⁹;
   —(CH₂)ᵣO(CH₂)ₚC₆H₄C(═NR⁸⁰)NR⁷⁸R⁷⁹; —(CH₂)ᵣO(CH₂)ₚC₆H₄C(═NOR⁵⁰)NR⁷⁸R⁷⁹;
   —(CH₂)ᵣO(CH₂)ₚC₆H₄C(═NNR⁷⁸R⁷⁹)NR⁷⁸R⁷⁹;
   —(CH₂)ᵣO(CH₂)ₚC₆H₄NR⁸⁰C(═NR⁸⁰)NR⁷⁸R⁷⁹; —(CH₂)ᵣS(CH₂)ₘNR⁷⁸R⁷⁹;
   —(CH₂)ᵣS(CH₂)ₘNR⁷⁷R⁸⁰; —(CH₂)ᵣS(CH₂)ₚC(═NR⁸⁰)NR⁷⁸R⁷⁹;
   —(CH₂)ᵣS(CH₂)ₚC(═NOR⁵⁰)NR⁷⁸R⁷⁹; —(CH₂)ᵣS(CH₂)ₚC(═NNR⁷⁸R⁷⁹)NR⁷⁸R⁷⁹;
   —(CH₂)ᵣS(CH₂)ₘNR⁸⁰C(═NR⁸⁰)NR⁷⁸R⁷⁹; —(CH₂)ᵣS(CH₂)ₘN═C(NR⁷⁸R⁸⁰)NR⁷⁹R⁸⁰;
   —(CH₂)ᵣS(CH₂)ₚC₆H₄CNR⁷⁸R⁷⁹; —(CH₂)ᵣS(CH₂)ₚC₆H₄C(═NR⁸⁰)NR⁷⁸R⁷⁹;
   —(CH₂)ᵣS(CH₂)ₚC₆H₄C(═NOR⁵⁰)NR⁷⁸R⁷⁹;
   —(CH₂)ᵣS(CH₂)ₚC₆H₄C(═NNR⁷⁸R⁷⁹)NR⁷⁸R⁷⁹;
   —(CH₂)ᵣS(CH₂)ₚC₆H₄NR⁸⁰C(═NR⁸⁰)NR⁷⁸R⁷⁹; —(CH₂)ₚNR⁸⁰COR⁶⁴; —(CH₂)ₚNR⁸⁰COR⁷⁷;
   —(CH₂)ₚNR⁸⁰CONR⁷⁸R⁷⁹; —(CH₂)ₚC₆H₄NR⁸⁰CONR⁷⁸R⁷⁹
R⁷⁵ is lower alkyl; lower alkenyl; or aryl-lower alkyl; or
R³³ and R⁷⁵ taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—; or
R⁷⁵ and R⁸² taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R⁷⁶ is H; lower alkyl; lower alkenyl; aryl-lower alkyl; —(CH₂)ₒOR⁷²; —(CH₂)ₒSR⁷²;
   —(CH₂)ₒNR³³R³⁴; —(CH₂)ₒOCONR³³R⁷⁵; —(CH₂)ₒNR²⁰CONR³³R⁸²;
   —(CH₂)ₒCOOR⁷⁵; —(CH₂)ₒCONR⁵⁸R⁵⁹; —(CH₂)ₒPO(OR⁶⁰)₂; —(CH₂)ₚSO₂R⁶²; or
   —(CH₂)ₒCOR⁶⁴;
R⁷⁷ is —C₆R⁶⁷R⁶⁸R⁶⁹R⁷⁰R⁷⁶; or a heteroaryl group of one of the formulae

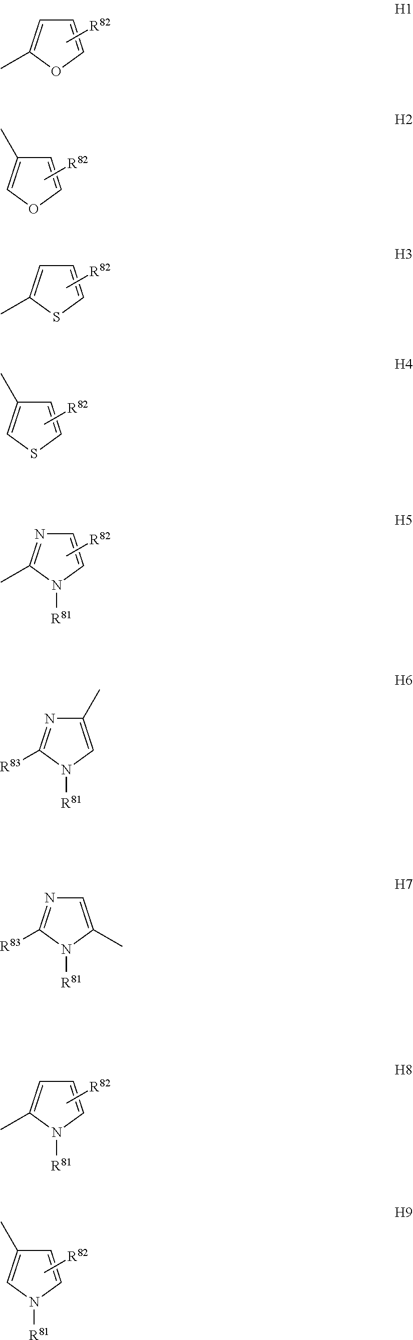

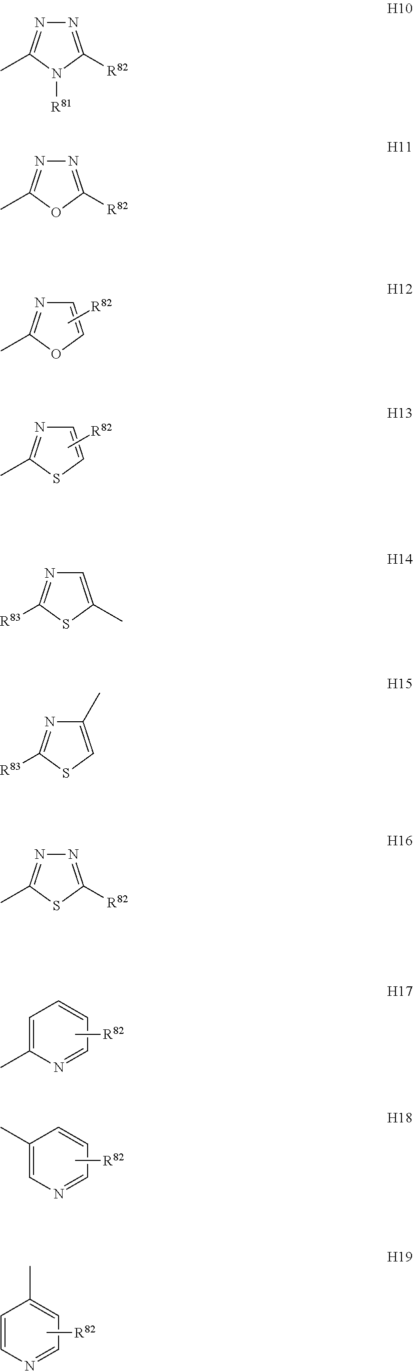

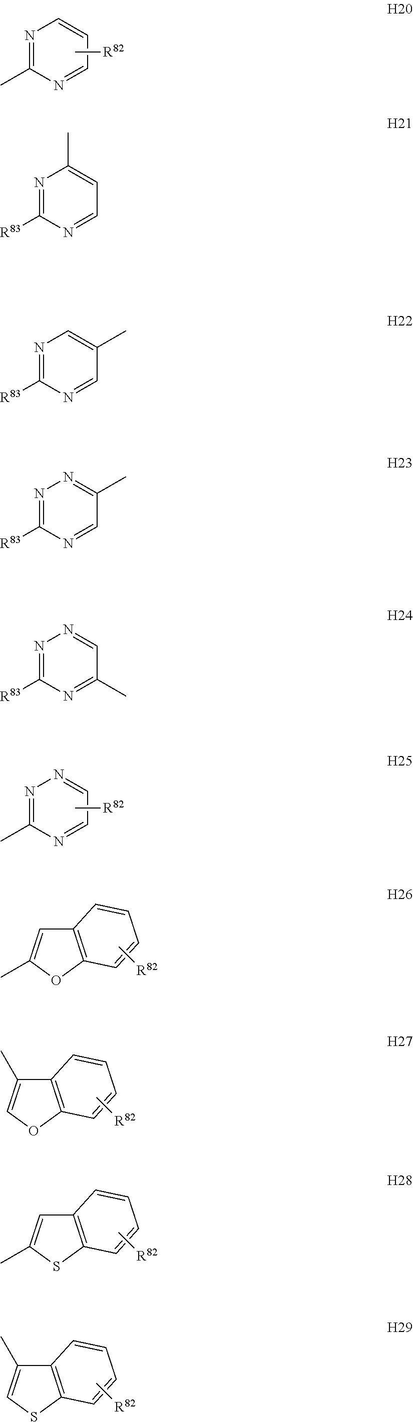

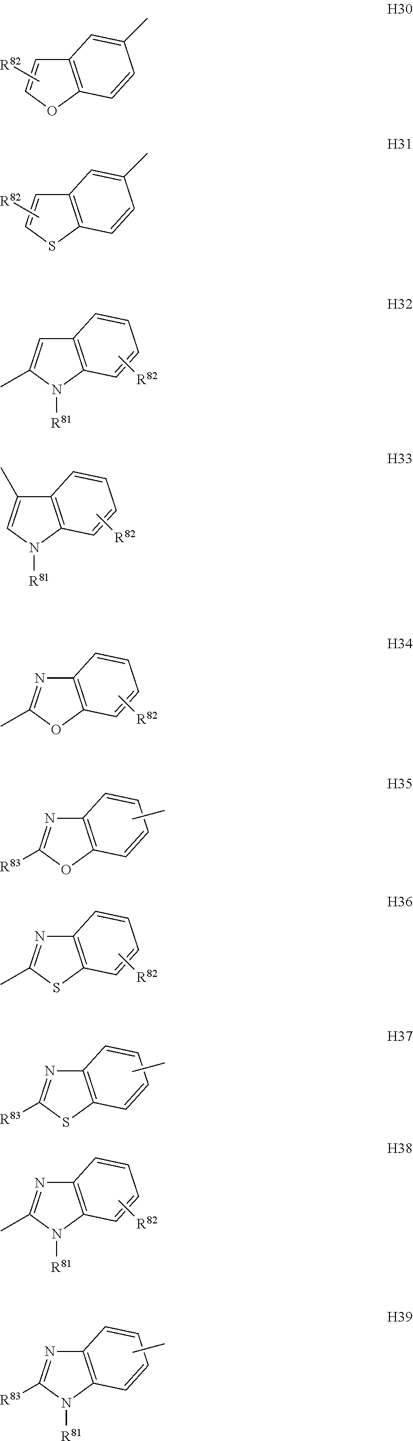

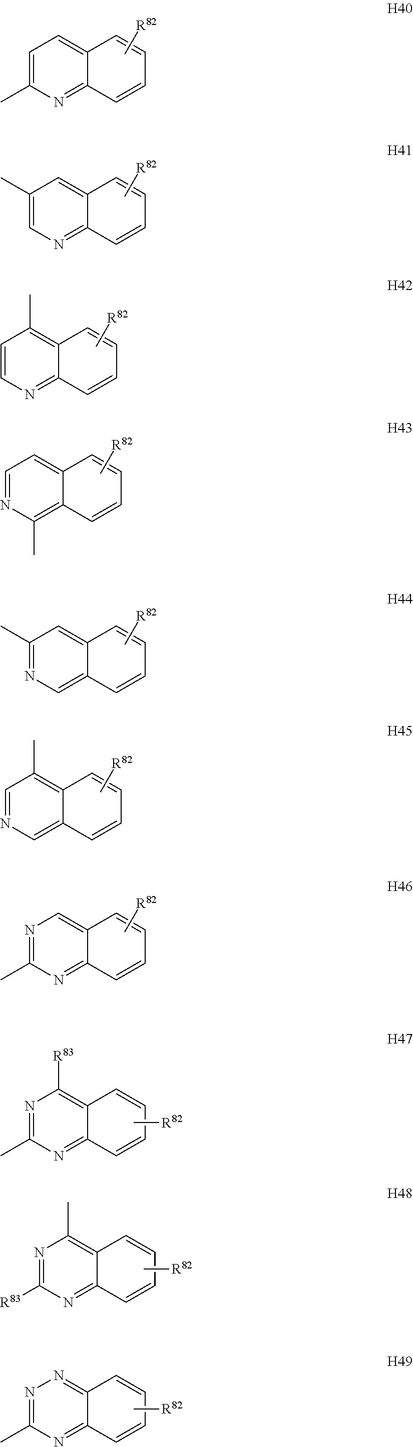

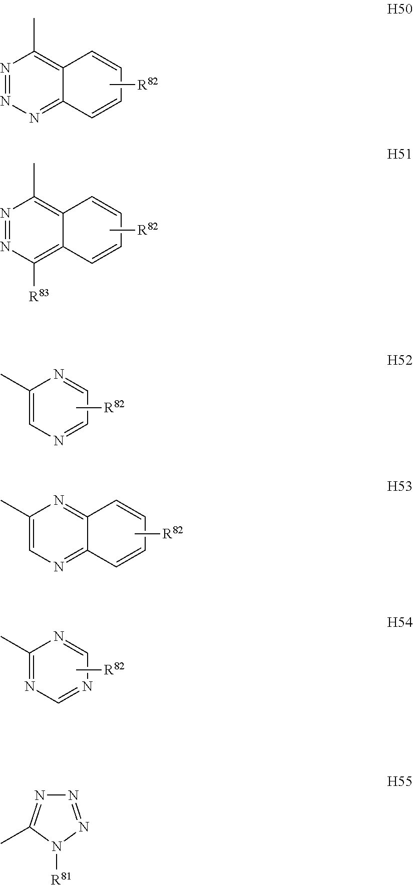

R⁷⁸ is H; lower alkyl; aryl; or aryl-lower alkyl; or
R⁷⁸ and R⁸² taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R⁷⁹ is H; lower alkyl; aryl; or aryl-lower alkyl; or
R⁷⁸ and R⁷⁹, taken together, can be —(CH₂)₂₋₇—; —(CH₂)₂O(CH₂)₂—; or —(CH₂)₂NR⁵⁷(CH₂)₂—;
R⁸⁰ is H; or lower alkyl;
R⁸¹ is H; lower alkyl; or aryl-lower alkyl;
R⁸² is H; lower alkyl; aryl; heteroaryl; or aryl-lower alkyl; or
R³³ and R⁸² taken together can form: —(CH₂)₂₋₆—; —(CH₂)₂O(CH₂)₂—; —(CH₂)₂S(CH₂)₂—; or
   —(CH₂)₂NR⁵⁷(CH₂)₂—;
R⁸³ is H; lower alkyl; aryl; or —NR⁷⁸R⁷⁹;
R⁸⁴ is —(CH₂)ₘ(CHR₆₁)ₛOH; —(CH₂)pCOOR₈₀; —(CH₂)ₘ(CHR₆₁)ₛSH; —(CH₂)ₚCONR⁷⁸R⁷⁹;
   —(CH₂)ₚNR⁸⁰CONR⁷⁸R⁷⁹; —(CH₂)ₚC₆H₄CONR⁷⁸R⁷⁹; or —(CH₂)ₚC₆H₄NR⁸⁰CONR⁷⁸R⁷⁹;
R⁸⁵ is lower alkyl; or lower alkenyl;
m is 2-4; o is 0-4; p is 1-4; q is 0-2; r is 1 or 2; s is 0 or 1;
   with the proviso that in said chain Z of n α-amino acid residues the amino acid residues in positions 1 to 8 are:
   P1: of type C, or of type D, or of type E, or of type F;
   P2: of type C, or of type F;
   P3: of type C, or of type D;
   P4: of type C, or of type D, or of type F, or the residue is Gly;
   P5: of type C, or of type D, or of type E, or of type F, or the residue is Gly or Pro;
   P6: of type C, or of type D; or the residue is Pro;
   P7: of type C, or of type D, or of type F;
   P8: of type C, or of type D, or of type E, or of type F; or
   P2 and P7, taken together, can form a group of type H; at P4 and P5 also D-isomers being possible;
      and pharmaceutically acceptable salts thereof.

In accordance with the present invention these β-hairpin peptidomimetics can be prepared by a process which comprises

(a) coupling an appropriately functionalized solid support with an appropriately N-protected derivative of that amino acid which in the desired end-product is in positions 3, 4 or 5, any functional group which may be present in said N-protected amino acid derivative being likewise appropriately protected;
(b) removing the N-protecting group from the product thus obtained;
(c) coupling the product thus obtained with an appropriately N-protected derivative of that amino acid which in the desired end-product is one position nearer the N-terminal amino acid residue, any functional group which may be present in said N-protected amino acid derivative being likewise appropriately protected;
(d) removing the N-protecting group from the product thus obtained;
(e) repeating steps (c) and (d) until the N-terminal amino acid residue has been introduced;
(f) coupling the product thus obtained with a compound of the general formula

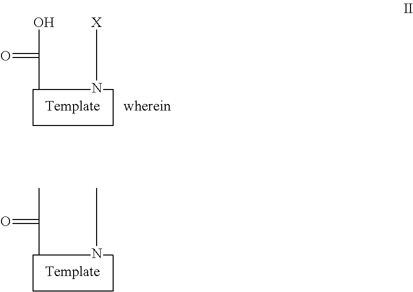

is as defined above and X is an N-protecting group or, alternatively, if

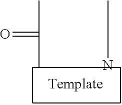

is to be group (a1), (a2) or (a3) above,
(fa) coupling the product obtained in step (e) with an appropriately N-protected derivative of an amino acid of the general formula

HOOC—B—H III

or

HOOC-A-H IV
wherein B and A are as defined above, any functional group which may be present in said N-protected amino acid derivative being likewise appropriately protected;
(fb) removing the N-protecting group from the product thus obtained; and
(fc) coupling the product thus obtained with an appropriately N-protected derivative of an amino acid of the above general formula IV or formula

HOOC—B3-H V
wherein B3 is as defined above,
and, respectively, formula III, any functional group which may be present in said N-protected amino acid derivative being likewise appropriately protected;
   (g) removing the N-protecting group from the product obtained in step (f) or (fc);
   (h) coupling the product thus obtained with an appropriately N-protected derivative of that amino acid which in the desired end-product is in position 8, any functional group which may be present in said N-protected amino acid derivative being likewise appropriately protected;
   (i) removing the N-protecting group from the product thus obtained;
   coupling the product thus obtained with an appropriately N-protected derivative of that amino acid which in the desired end-product is one position farther away from position 8, any functional group which may be present in said N-protected amino acid derivative being likewise appropriately protected;
   (k) removing the N-protecting group from the product thus obtained;
   (l) repeating steps (j) and (k) until all amino acid residues have been introduced;
   (m) if desired, selectively deprotecting one or several protected functional group(s) present in the molecule and appropriately substituting the reactive group(s) thus liberated;
   (n) if desired, forming an interstrand linkage between side-chains of appropriate amino acid residues at positions 2 and 7;
   (o) detaching the product thus obtained from the solid support;
   (p) cyclizing the product cleaved from the solid support;
   (q) removing any protecting groups present on functional groups of any members of the chain of amino acid residues and, if desired, any protecting group(s) which may in addition be present in the molecule; and
   (r) if desired, converting the product thus obtained into a pharmaceutically acceptable salt or converting a pharmaceutically acceptable, or unacceptable, salt thus obtained into the corresponding free compound of formula I or into a different, pharmaceutically acceptable, salt.

Alternatively, the peptidomimetics of the present invention can be prepared by

(a′) coupling an appropriately functionalized solid support with a compound of the general formula

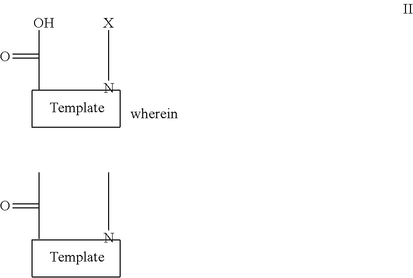

is as defined above and X is an N-protecting group or, alternatively, if

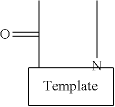

is to be group (a1), (a2) or (a3) above,
(a′a) coupling said appropriately functionalized solid support with an appropriately
N-protected derivative of an amino acid of the general formula

HOOC—B—H III

or

HOOC-A-H IV
wherein B and A are as defined above, any functional group which may be present in said N-protected amino acid derivative being likewise appropriately protected;
(a′b) removing the N-protecting group from the product thus obtained; and
(a′c) coupling the product thus obtained with an appropriately N-protected derivative of an amino acid of the above general formula IV or formula

HOOC—B3-H V
wherein B3 is as defined above,
and, respectively, formula III, any functional group which may be present in said N-protected amino acid derivative being likewise appropriately protected;
   (b′) removing the N-protecting group from the product obtained in step (a′) or (a′c)
   (c′) coupling the product thus obtained with an appropriately N-protected derivative of that amino acid which in the desired end-product is in position 8, any functional group which may be present in said N-protected amino acid derivative being likewise appropriately protected;
   (d′) removing the N-protecting group from the product thus obtained;
   (e′) coupling the product thus obtained with an appropriately N-protected derivative of that amino acid which in the desired end-product is one position farther away from position 8, any functional group which may be present in said N-protected amino acid derivative being likewise appropriately protected;
   (f′) removing the N-protecting group from the product thus obtained;
   (g′) repeating steps (e′) and (f′) until all amino acid residues have been introduced;
   (h′) if desired, selectively deprotecting one or several protected functional group(s) present in the molecule and appropriately substituting the reactive group(s) thus liberated;
   (i′) if desired forming an interstrand linkage between side-chains of appropriate amino acid residues at positions 2 and 7;
   (j′) detaching the product thus obtained from the solid support;
   (k′) cyclizing the product cleaved from the solid support;
   (l′) removing any protecting groups present on functional groups of any members of the chain of amino acid residues and, if desired, any protecting group(s) which may in addition be present in the molecule; and
   (m′) if desired, converting the product thus obtained into a pharmaceutically acceptable salt or converting a pharmaceutically acceptable, or unacceptable, salt thus obtained into the corresponding free compound of formula I or into a different, pharmaceutically acceptable, salt.

The peptidomimetics of the present invention can also be enantiomers of the compounds of formula I. These enantiomers can be prepared by a modification of the above processes in which enantiomers of all chiral starting materials are used.

As used in this description, the term “alkyl”, taken alone or in combinations, designates saturated, straight-chain or branched hydrocarbon radicals having up to 24, preferably up to 12, carbon atoms. Similarly, the term “alkenyl” designates straight chain or branched hydrocarbon radicals having up to 24, preferably up to 12, carbon atoms and containing at least one or, depending on the chain length, up to four olefinic double bonds. The term “lower” designates radicals and compounds having up to 6 carbon atoms. Thus, for example, the terms “lower alkyl” and “lower cycloalkyl” designate saturated, straight-chain or branched and, respectively cyclic hydrocarbon radicals having up to 6 carbon atoms, such as methyl, ethyl, n-propyl, isopropyl, n-butyl, sec.-butyl, isobutyl, tert.-butyl, cyclopentyl, cyclohexyl and the like. The term “aryl” designates aromatic carbocyclic hydrocarbon radicals containing one or two six-membered rings, such as phenyl or naphthyl, which may be substituted by up to three substituents such as Br, Cl, F, CF₃, NO₂, lower alkyl or lower alkenyl. The term “heteroaryl” designates aromatic heterocyclic radicals containing one or two five- and/or six-membered rings, at least one of them containing up to three heteroatoms selected from the group consisting of O, S and N and said ring(s) being optionally substituted; representative examples of such optionally substituted heteroaryl radicals are indicated hereinabove in connection with the definition of R⁷⁷.

The structural element -A-CO— designates amino acid building blocks which in combination with the structural element —B—CO— form templates (a1) and (a2). Similarly, the structural element —B3-CO-designates amino acid building blocks which in combination with the structural element —B—CO— form template (a3). Templates (a) through (p) constitute building blocks which have an N-terminus and a C-terminus oriented in space in such a way that the distance between those two groups may lie between 4.0-5.5A. A peptide chain Z is linked to the C-terminus and the N-terminus of the templates (a) through (p) via the corresponding N- and C-termini so that the template and the chain form a cyclic structure such as that depicted in formula I. In a case as here where the distance between the N- and C-termini of the template lies between 4.0-5.5A the template will induce the H-bond network necessary for the formation of a β-hairpin conformation in the peptide chain Z. Thus template and peptide chain form a β-hairpin mimetic.

The β-hairpin conformation is highly relevant for the agonizing or antagonizing activity against urotensin II as well as the inhibition of the STAT6/NCoA-1 interaction of the β-hairpin mimetics of the present invention. The β-hairpin stabilizing conformational properties of the templates (a) through (p) play a key role not only for the selective activities described above but also for the synthesis process defined hereinabove, as incorporation of the templates at the beginning or near the middle of the linear protected peptide precursors enhances cyclization yields significantly.

Building blocks A1-A69 and A105 belong to a class of amino acids wherein the N-terminus is a secondary amine forming part of a ring. Among the genetically encoded amino acids only proline falls into this class. The configuration of building block A1 through A69 and A105 is (D), and they are combined with a building block —B—CO— of (L)-configuration. Preferred combinations for templates (a1) are -^{D}A1-CO-^{L}B—CO— to -^{D}A69-CO—^{L}B—CO— and ^{D}A105-CO—^{L}B—CO—. Thus, for example, ^{D}Pro-^{L}Pro constitutes the prototype of templates (a1). Less preferred, but possible are combinations ^{L}B—CO-^{D}A1-CO— to ^{L}B—CO-^{D}A69-CO— and ^{L}B—CO-^{D}A105-CO— forming templates (a2). Thus, for example, ^{L}Pro-^{D}Pro constitutes the prototype of template (a2). Template (a3) consists of the combination -^{D}B3-CO—^{L}B—CO—, ^{D}Ser-^{L}Pro and ^{D}Glu-^{L}Pro constituting prototypes of template (a3).

It will be appreciated that building blocks -A1-CO— to -A69-CO— and A105-CO— in which A has (D)-configuration, are carrying a group R¹ at the α-position to the N-terminus. The preferred values for R¹ are H and lower alkyl with the most preferred values for R¹ being H and methyl. It will be recognized by those skilled in the art, that A1-A69 and A105 are shown in (D)-configuration which, for R¹ being H and methyl, corresponds to the (R)-configuration. Depending on the priority of other values for R¹ according to the Cahn, Ingold and Prelog-rules, this configuration may also have to be expressed as (S).

In addition to R¹ building blocks -A1-CO— to -A69-CO— and A105-CO— can carry an additional substituent designated as R² to R¹⁷ or R⁷⁷. This additional substituent can be H, and if it is other than H, it is preferably a small to medium-sized aliphatic, aromatic or heteroaromatic group. Examples of preferred values for R² to R¹⁷ are:
R²: H; lower alkyl; lower alkenyl; (CH₂)ₘOR⁵⁵ (where R⁵⁵: lower alkyl; or lower alkenyl); (CH₂)ₘSR⁵⁶ (where R⁵⁶: lower alkyl; or lower alkenyl); (CH₂)ₘNR³³R³⁴ (where R³³: lower alkyl; or lower alkenyl; R³⁴: H; or lower alkyl; R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; R⁵⁷: H; or lower alkyl); (CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R³: H; lower alkyl; lower alkenyl; —(CH₂)ₘOR⁵⁵ (where R⁵⁵: lower alkyl; or lower alkenyl); —(CH₂)ₘSR⁵⁶ (where R⁵⁶: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); (CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R⁴: H; lower alkyl; lower alkenyl; —(CH₂)ₘOR⁵⁵ (where R⁵⁵: lower alkyl; or lower alkenyl); —(CH₂)ₘSR⁵⁶ (where R⁵⁶: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴(where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).

R⁵: lower alkyl; lower alkenyl; —(CH₂)ₒR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; R⁵⁷: where H; or lower alkyl); (CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); (CH₂)ₒN(R²⁰)COR⁶⁴(where: R²⁰: H; or lower alkyl; R⁶⁴: alkyl; alkenyl; aryl; and aryl-lower alkyl; heteroaryl-lower alkyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R⁶: H; lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R⁷: lower alkyl; lower alkenyl; —(CH₂)_{q}OR⁵⁵ (where R⁵⁵: lower alkyl; or lower alkenyl); —(CH₂)_{q}SR⁵⁶ (where R⁵⁶: lower alkyl; or lower alkenyl); —(CH₂)_{q}NR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}OCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); (CH₂)_{q}NR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}N(R²⁰)COR⁶⁴(where: R²⁰: H; or lower alkyl; R⁶⁴: lower alkyl; or lower alkenyl); —(CH₂)ᵣCOOR⁵⁷ (where R⁵⁷: lower alkyl; or lower alkenyl); —(CH₂)_{q}CONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ᵣPO(OR⁶⁰)₂ (where R⁶⁰: lower alkyl; or lower alkenyl); (CH₂)ᵣSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R^{S}: H; F; Cl; CF₃; lower alkyl; lower alkenyl; —(CH₂)ₒOR⁵⁵ (where R⁵⁵: lower alkyl; or lower alkenyl); (CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).

R⁹: lower alkyl; lower alkenyl; —(CH₂)ₒR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴(where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).

R¹⁰: lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴(where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R¹¹: H; lower alkyl; lower alkenyl; —(CH₂)ₘOR⁵⁵ (where R⁵⁵: lower alkyl; or lower alkenyl); —(CH₂)ₘSR⁵⁶ (where R⁵⁶: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).

R¹²: H; lower alkyl; lower alkenyl; —(CH₂)ₘOR⁵⁵ (where R⁵⁵: lower alkyl; or lower alkenyl); —(CH₂)ₘSR⁵⁶ (where R⁵⁶: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ᵣCOOR⁵⁷ (where R⁵⁷: lower alkyl; or lower alkenyl); —(CH₂)ᵣCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ᵣPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R¹³: lower alkyl; lower alkenyl; —(CH₂)_{q}OR⁵⁵ (where R⁵⁵: lower alkyl; or lower alkenyl); —(CH₂)_{q}SR⁵⁶ (where R⁵⁶: lower alkyl; or lower alkenyl); —(CH₂)_{q}NR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}OCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}NR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}N(R²⁰)COR⁶⁴ (where: R²⁰: H; or lower alkyl; R⁶⁴: lower alkyl; or lower alkenyl); —(CH₂)ᵣCOO⁵⁷ (where R⁵⁷: lower alkyl; or lower alkenyl); —(CH₂)_{q}CONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ᵣPO(OR⁶⁰)₂ (where R⁶⁰: lower alkyl; or lower alkenyl); —(CH₂)ᵣSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R¹⁴: H; lower alkyl; lower alkenyl; —(CH₂)ₘOR⁵⁵ (where R⁵⁵: lower alkyl; or lower alkenyl); —(CH₂)ₘSR⁵⁶ (where R⁵⁶: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); —(CH₂)_{q}C₆H₄R^{S} (where R^{S}: H; F; Cl; CF₃; lower alkyl; lower alkenyl; or lower alkoxy).
R¹⁵: lower alkyl; lower alkenyl; —(CH₂)ₒR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); (CH₂)ₒN(R²⁰)COR⁶⁴ (where: R²⁰: H; or lower alkyl; R⁶⁴: lower alkyl; or lower alkenyl); particularly favoured are NR²⁰COlower alkyl (R²⁰═H; or lower alkyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R¹⁶: lower alkyl; lower alkenyl; —(CH₂)ₒR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R¹⁷: lower alkyl; lower alkenyl; —(CH₂)_{q}OR⁵⁵ (where R⁵⁵: lower alkyl; or lower alkenyl); —(CH₂)_{q}SR⁵⁶ (where R⁵⁶: lower alkyl; or lower alkenyl); —(CH₂)_{q}NR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}OCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}NR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)_{q}N(R²⁰)COR⁶⁴(where: R²⁰: H; or lower alkyl; R⁶⁴: lower alkyl; or lower alkenyl); —(CH₂)_{q}COOR⁵⁷ (where R⁵⁷: lower alkyl; or lower alkenyl); —(CH₂)_{q}CONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ᵣ(OR⁶⁰)₂ (where R⁶⁰: lower alkyl; or lower alkenyl); —(CH₂)ᵣSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).

Among the building blocks A1 to A69 and A105 the following are preferred: A5 with R² being H, A8, A22, A25, A38 with R² being H, A42, A47, and A50 and A105. Most preferred are building blocks of type A8′:

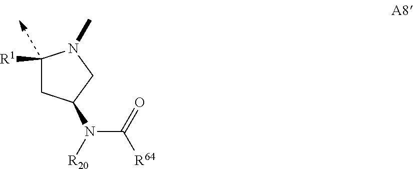

wherein R²⁰ is H or lower alkyl; and R⁶⁴ is alkyl; alkenyl; aryl; aryl-lower alkyl; or heteroaryl-lower alkyl; especially those wherein R⁶⁴ is n-hexyl (A8′-1); n-heptyl (A8′-2); 4-(phenyl)benzyl (A8′-3); diphenylmethyl (A8′-4); 3-amino-propyl (A8′-5); 5-amino-pentyl (A8′-6); methyl (A8′-7); ethyl (A8′-8); isopropyl (A8′-9); isobutyl (A8′-10); n-propyl (A8′-11); cyclohexyl (A8′-12); cyclohexylmethyl (A8′-13); n-butyl (A8′-14); phenyl (A8′-15); benzyl (A8′-16); (3-indolyl)methyl (A8′-17); 2-(3-indolyl)ethyl (A8′-18); (4-phenyl)phenyl (A8′-19); and n-nonyl (A8′-20).

Building block A70 belongs to the class of open-chain α-substituted α-amino acids, building blocks A71 and A72 to the corresponding (3-amino acid analogues and building blocks A73-A104 to the cyclic analogues of A70. Such amino acid derivatives have been shown to constrain small peptides in well defined reverse turn or U-shaped conformations (C. M. Venkatachalam, Biopolymers 1968, 6, 1425-1434; W. Kabsch, C. Sander, Biopolymers 1983, 22, 2577). Such building blocks or templates are ideally suited for the stabilization of β-hairpin conformations in peptide loops (D. Obrecht, M. Altorfer, J. A. Robinson, “Novel Peptide Mimetic Building Blocks and Strategies for Efficient Lead Finding”, Adv. Med. Chem. 1999, Vol. 4, 1-68; P. Balaram, “Non-standard amino acids in peptide design and protein engineering”, Curr. Opin. Struct. Biol. 1992, 2, 845-851; M. Crisma, G. Valle, C. Toniolo, S. Prasad, R. B. Rao, P. Balaram, “β-turn conformations in crystal structures of model peptides containing α,α-disubstituted amino acids”, Biopolymers 1995, 35, 1-9; V. J. Hruby, F. Al-Obeidi, W. Kazmierski, Biochem. J. 1990, 268, 249-262).

It has been shown that both enantiomers of building blocks -A70-CO— to A104-CO— in combination with a building block —B—CO— of L-configuration can efficiently stabilize and induce β-hairpin conformations (D. Obrecht, M. Altorfer, J. A. Robinson, “Novel Peptide Mimetic Building Blocks and Strategies for Efficient Lead Finding”, Adv. Med. Chem. 1999, Vol. 4, 1-68; D. Obrecht, C. Spiegler, P. Schönholzer, K. Müller, H. Heimgartner, F. Stierli, Helv. Chim. Acta 1992, 75, 1666-1696; D. Obrecht, U. Bohdal, J. Daly, C. Lehmann, P. Schönholzer, K. Müller, Tetrahedron 1995, 51, 10883-10900; D. Obrecht, C. Lehmann, C. Ruffieux, P. Schönholzer, K. Müller, Helv. Chim. Acta 1995, 78, 1567-1587; D. Obrecht, U. Bohdal, C. Broger, D. Bur, C. Lehmann, R. Ruffieux, P. Schönholzer, C. Spiegler, Helv. Chim. Acta 1995, 78, 563-580; D. Obrecht, H. Karajiannis, C. Lehmann, P. Schönholzer, C. Spiegler, Helv. Chim. Acta 1995, 78, 703-714).

Thus, for the purposes of the present invention templates (a1) can also consist of -A70-CO— to A104-CO— where building block A70 to A104 is of either (D)- or (L)-configuration, in combination with a building block —B—CO— of (L)-configuration. Preferred values for R²⁰ in A70 to A104 are H or lower alkyl with methyl being most preferred. Preferred values for R¹⁸, R¹⁹ and R²¹-R²⁹ in building blocks A70 to A104 are the following:
R¹⁸: lower alkyl.
R¹⁹: lower alkyl; lower alkenyl; —(CH₂)ₚOR⁵⁵ (where R⁵⁵: lower alkyl; or lower alkenyl); —(CH₂)ₚSR⁵⁶ (where R⁵⁶: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₚCOOR⁵⁷ (where R⁵⁷: lower alkyl; or lower alkenyl); —(CH₂)ₚCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₚSO₂R⁶² (where R⁶²: lower alkyl; or lower alkenyl); or —(CH₂)ₒC₆H₄R^{S} (where R^{S}: H; F; Cl; CF₃; lower alkyl; lower alkenyl; or lower alkoxy).
R²¹: H; lower alkyl; lower alkenyl; —(CH₂)ₚOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); (CH₂)ₒSO₂R⁶² (where R⁶²: lower alkyl; or lower alkenyl); or (CH₂)_{q}C₆H₄R^{S} (where R^{S}: H; F; Cl; CF₃; lower alkyl; lower alkenyl; or lower alkoxy).
R²²: lower alkyl; lower alkenyl; —(CH₂)ₒR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴(where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF; lower alkyl; lower alkenyl; or lower alkoxy).
R²³: H; lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); particularly favoured are NR²⁰COlower alkyl (R²⁰═H; or lower alkyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy);
R²⁴: lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); particularly favoured are NR²⁰COlower alkyl (R²⁰═H; or lower alkyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy);
R²⁵: H; lower alkyl; lower alkenyl; —(CH₂)ₘOR⁵⁵ (where R⁵⁵: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R²⁶: H; lower alkyl; lower alkenyl; —(CH₂)ₘOR⁵⁵ (where R⁵⁵: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴(where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
Alternatively, R²⁵ and R²⁶ taken together can be —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl).
R²⁷: H; lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R²⁸: lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴(where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R²⁹: lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴(where: R²⁰: H; or lower alkyl; R⁶⁴: lower alkyl; or lower alkenyl); particularly favored are NR²⁰COlower-alkyl (R²⁰═H; or lower alkyl); —(CH₂)₂COOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).

For templates (b) to (p), such as (b1) and (c1), the preferred values for the various symbols are the following:
R^{S}: H; F; Cl; CF₃; lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; or lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R²⁰: H; or lower alkyl.
R³⁰: H, methyl.
R³¹: H; lower alkyl; lower alkenyl; —(CH₂)ₚ0R⁵⁵ (where R⁵⁵: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)ᵣC₆H₄R^{S} (where R^{S}: H; F; Cl; CF₃; lower alkyl; lower alkenyl; or lower alkoxy); most preferred is —CH₂CONR⁵⁸R⁵⁹ (R⁵⁸: H; or lower alkyl; R⁵⁹: lower alkyl; or lower alkenyl).
R³²: H, methyl.
R³³: lower alkyl; lower alkenyl; —(CH₂)ₘOR⁵⁵ (where R⁵⁵: lower alkyl; or lower alkenyl); —(CH₂)ₘNR³⁴R⁶³ (where R³⁴: lower alkyl; or lower alkenyl; R⁶³: H; or lower alkyl; or R³⁴ and R⁶³ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); (CH₂)ₘOCONR⁷⁵R⁸²(where R⁷⁵: lower alkyl; or lower alkenyl; R⁸²: H; or lower alkyl; or R⁷⁵ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR⁷⁸R⁸² (where R²⁰: H; or lower lower alkyl; R⁷⁸: H; or lower alkyl; or lower alkenyl; R⁸²: H; or lower alkyl; or R⁷⁸ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl).
R³⁴: H; or lower alkyl.
R³⁵: H; lower alkyl; lower alkenyl; —(CH₂)ₘOR⁵⁵ (where R⁵⁵: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or
   —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl).
R³⁶: lower alkyl; lower alkenyl; or aryl-lower alkyl.
R³⁷: H; lower alkyl; lower alkenyl; —(CH₂)ₚOR⁵⁵ (where R⁵⁵: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alky; or lower alkenyl); or —(CH₂)_{q}C₆H₄R^{S} (where R^{S}: H; F; Cl; CF₃; lower alkyl; lower alkenyl; or lower alkoxy).
R³⁸: H; lower alkyl; lower alkenyl; —(CH₂)ₛOR⁵⁵ (where R⁵⁵: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁸ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R³⁹: H; lower alkyl; lower alkenyl; —(CH₂)ₘOR⁵⁵ (where R⁵⁵: lower alkyl; or lower alkenyl); —(CH₂)ₘN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl).
R⁴⁰: lower alkyl; lower alkenyl; or aryl-lower alkyl.
R⁴¹: H; lower alkyl; lower alkenyl; —(CH₂)ₚOR⁵⁵ (where R⁵⁵: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alky; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R⁴²: H; lower alkyl; lower alkenyl; —(CH₂)ₚOR⁵⁵ (where R⁵⁵: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl, or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R⁴³: H; lower alkyl; lower alkenyl; —(CH₂)ₘOR⁵⁵ (where R⁵⁵: lower alkyl; or lower alkenyl); —(CH₂)ₘSR⁵⁶ (where R⁵⁶: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒPO(OR⁶⁰)₂ (where R⁶⁰: lower alkyl; or lower alkenyl); —(CH₂)ₒSO₂R⁶² (where R⁶²: lower alkyl; or lower alkenyl); or —(CH₂)_{q}C₆H₄R^{S} (where R^{S}: H; F; Cl; CF₃; lower alkyl; lower alkenyl; or lower alkoxy).
R⁴⁴: lower alkyl; lower alkenyl; —(CH₂)ₚOR⁵⁵ (where R⁵⁵: lower alkyl; or lower alkenyl); —(CH₂)ₚSR⁵⁶ (where R⁵⁶: lower alkyl; or lower alkenyl); —(CH₂)ₚNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚOCONR³³R⁷⁵ (where R³³: H; or lower alkyl; or lower alkenyl; R⁷⁵: lower alkyl; or R³³ and R⁷⁸ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₚN(R²⁰)COR⁶⁴ (where: R²⁰: H; or lower alkyl; R⁶⁴: lower alkyl; or lower alkenyl); —(CH₂)ₚCOOR⁵⁷ (where R⁵⁷: lower alkyl; or lower alkenyl); —(CH₂)ₚCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or —(CH₂)ₒC₆H₄R^{S} (where R^{S}: H; F; Cl; CF₃; lower alkyl; lower alkenyl; or lower alkoxy).
R⁴⁵: H; lower alkyl; lower alkenyl; —(CH₂)ₒOR⁵⁵ (where R⁵⁵: lower alkyl; or lower alkenyl); —(CH₂)ₒSR⁵⁶ (where R⁵⁶: lower alkyl; or lower alkenyl); —(CH₂)ₒNR³³R³⁴ (where R³³: lower alkyl; or lower alkenyl; R³⁴: H; or lower alkyl; or R³³ and R³⁴ taken together form:
   —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₛOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₒN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or —(CH₂)ₛC₆H₄R^{S} (where R^{S}: H; F; Cl; CF₃; lower alkyl; lower alkenyl; or lower alkoxy).
R⁴⁶: H; lower alkyl; lower alkenyl; —(CH₂)ₛOR⁵⁵ (where R⁵⁵: lower alkyl; or lower alkenyl); —(CH₂)ₛSR⁵⁶ (where R⁵⁶: lower alkyl; or lower alkenyl); —(CH₂)ₛNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₛOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₛNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₛN(R²⁰)COR⁶⁴ (where: R²⁰: H; or lower alkyl; R⁶⁴: lower alkyl; or lower alkenyl); —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or —(CH₂)ₛC₆H₄R^{S} (where R^{S}: H; F; Cl; CF₃; lower alkyl; lower alkenyl; or lower alkoxy).
R⁴⁷: H; or OR⁵⁵ (where R⁵⁵: lower alkyl; or lower alkenyl).
R⁴⁸: H; or lower alkyl.
R⁴⁹: H; lower alkyl; —(CH₂)ₒCOOR⁵⁷ (where R⁵⁷: lower alkyl; or lower alkenyl); —(CH₂)ₒCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or (CH₂)ₛC₆H₄R^{S} (where R^{S}: H; F; Cl; CF₃; lower alkyl; lower alkenyl; or lower alkoxy).
R⁵⁰: H; methyl.
R⁵¹: H; lower alkyl; lower alkenyl; —(CH₂)ₘOR⁵⁵ (where R⁵⁵: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); (CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₚCOOR⁵⁷ (where R⁵⁷: lower alkyl; or lower alkenyl); —(CH₂)ₚCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or —(CH₂)ᵣC₆H₄R^{S} (where R^{S}: H; F; Cl; CF₃; lower alkyl; lower alkenyl; or lower alkoxy).
R⁵²: H; lower alkyl; lower alkenyl; —(CH₂)ₘOR⁵⁵ (where R⁵⁵: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₚCOOR⁵⁷ (where R⁵⁷: lower alkyl; or lower alkenyl); —(CH₂)ₚCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or —(CH₂)ᵣC₆H₄R^{S} (where R^{S}: H; F; Cl; CF₃; lower alkyl; lower alkenyl; or lower alkoxy).
R⁵³: H; lower alkyl; lower alkenyl; —(CH₂)ₘOR⁵⁵ (where R⁵⁵: lower alkyl; or lower alkenyl); —(CH₂)ₘNR³³R³⁴ (where R³³: lower alkyl; or lower alkenyl; R³⁴: H; or lower alkyl; or R³³ and R³⁴ taken together form: —(CH₂)₂₋₆—; —(CH₂)₂O(CH₂)₂—; —(CH₂)₂S(CH₂)₂—; or
   —(CH₂)₂NR⁵⁷(CH₂)₂—; where R⁵⁷: H; or lower alkyl); —(CH₂)ₘOCONR³³R⁷⁵ (where R³³: H; or lower alkyl; or lower alkenyl; R⁷⁵: lower alkyl; or R³³ and R⁷⁵ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘNR²⁰CONR³³R⁸² (where R²⁰: H; or lower lower alkyl; R³³: H; or lower alkyl; or lower alkenyl; R⁸²: H; or lower alkyl; or R³³ and R⁸² taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); —(CH₂)ₘN(R²⁰)COR⁶⁴ (where: R²⁰: H; or lower alkyl; R⁶⁴: lower alkyl; or lower alkenyl); —(CH₂)ₚCOOR⁵⁷ (where R⁵⁷: lower alkyl; or lower alkenyl); —(CH₂)ₚCONR⁵⁸R⁵⁹ (where R⁵⁸: lower alkyl; or lower alkenyl; and R⁵⁹: H; lower alkyl; or R⁵⁸ and R⁵⁹ taken together form: —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl); or —(CH₂)_{q}C₆H₄R^{S} (where R^{S}: H; F; Cl; CF₃; lower alkyl; lower alkenyl; or lower alkoxy).
R⁵⁴: lower alkyl; lower alkenyl; or aryl-lower alkyl.

Among the building blocks A70 to A104 the following are preferred: A74 with R²² being H, A75, A76, A77 with R²² being H, A78 and A79.

The building block —B—CO— within templates (a1), (a2) and (a3) designates an L-amino acid residue. Preferred values for B are: —NR²⁰CH(R⁷¹)— and enantiomers of groups A5 with R² being H, A8, A22, A25, A38 with R² being H, A42, A47, and A50. Most preferred are
Ala L-Alanine
Arg L-Arginine
Asn L-Asparagine
Cys L-Cysteine
Gln L-Glutamine
Gly Glycine
H is L-Histidine
Ile L-Isoleucine
Leu L-Leucine
Lys L-Lysine
Met L-Methionine
Phe L-Phenylalanine
Pro L-Proline
Ser L-Serine
Thr L-Threonine
Trp L-Tryptophan
Tyr L-Tyrosine
Val L-Valine
Cit L-Citrulline
Orn L-Ornithine
tBuA L-t-Butylalanine
Sar L-Sarcosine
t-BuG L-tert.-Butylglycine
4AmPhe L-para-Aminophenylalanine
3AmPhe L-meta-Aminophenylalanine
2AmPhe L-ortho-Aminophenylalanine
Phe(mC(NH₂)═NH) L-meta-Amidinophenylalanine
Phe(pC(NH₂)═NH) L-para-Amidinophenylalanine
Phe(mNHC(NH₂)═NH) L-meta-Guanidinophenylalanine
Phe(pNHC(NH₂)═NH) L-para-Guanidinophenylalanine
Phg L-Phenylglycine
Cha L-Cyclohexylalanine
C₄al L-3-Cyclobutylalanine
C₅al L-3-Cyclopentylalanine
Nle L-Norleucine
2-Nal L-2-Naphthylalanine
1-Nal L-1-Naphthylalanine
4Cl-Phe L-4-Chlorophenylalanine
3Cl-Phe L-3-Chlorophenylalanine
2Cl-Phe L-2-Chlorophenylalanine
3,4Cl₂Phe L-3,4-Dichlorophenylalanine
4F-Phe L-4-Fluorophenylalanine
3F-Phe L-3-Fluorophenylalanine
2F-Phe L-2-Fluorophenylalanine
Tic L-1,2,3,4-Tetrahydroisoquinoline-3-carboxylic acid
Thi L-β-2-Thienylalanine
Tza L-2-Thiazolylalanine
Mso L-Methionine sulfoxide
AcLys L-N-Acetyllysine
Dpr L-2,3-Diaminopropionic acid
A₂Bu L-2,4-Diaminobutyric acid
Dbu (2S,3S)-2,3-Diaminobutyric acid
Abu γ-Aminobutyric acid (GABA)
Aha ε-Aminohexanoic acid
Aib α-Aminoisobutyric acid
Y(Bzl) L-O-Benzyltyrosine
Bip L-Biphenylalanine
S(Bzl) L-O-Benzylserine
T(Bzl) L-O-Benzylthreonine
hCha L-Homo-cyclohexylalanine
hCys L-Homo-cysteine
hSer L-Homo-serine
hArg L-Homo-arginine
hPhe L-Homo-phenylalanine
Bpa L-4-Benzoylphenylalanine
Pip L-Pipecolic acid
OctG L-Octylglycine
MePhe L-N-Methylphenylalanine
MeNle L-N-Methylnorleucine
MeAla L-N-Methylalanine
MeIle L-N-Methylisoleucine
MeVal L-N-Methvaline
MeLeu L-N-Methylleucine
4Hyp1 (4S)-L-Hydroxyproline
4Hyp2 (4R)-L-Hydroxyproline
4 Mp1 (4S)-L-Mercaptoproline
4 Mp2 (4R)-L-Mercaptoproline
Oic (3aS, 7aS)-L-1-Octahydro-1H-indole-2-carboxylic acid

In addition, preferred values for B also include groups of type A8″ of (L)-configuration:

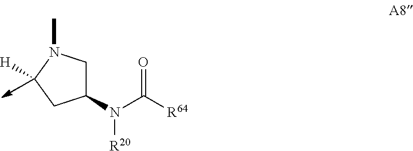

wherein R²⁰ is H or lower alkyl and R⁶⁴ is alkyl; alkenyl; —[(CH₂)ᵤ—X]ₜ—CH₃ (where X is —O—; —NR²⁰—, or —S—; u=1-3, and t=1-6), aryl; aryl-lower alkyl; or heteroaryl-lower alkyl; especially those wherein R⁶⁴ is n-hexyl (A8″-21); n-heptyl (A8″-22); 4-(phenyl)benzyl (A8″-23); diphenylmethyl (A8″-24); 3-amino-propyl (A8″-25); 5-amino-pentyl (A8″-26); methyl (A8″-27); ethyl (A8″-28); isopropyl (A8″-29); isobutyl (A8″-30); n-propyl (A8″-31); cyclohexyl (A8″-32); cyclohexylmethyl (A8″-33); n-butyl (A8″-34); phenyl (A8″-35); benzyl (A8″-36); (3-indolyl)methyl (A8″-37); 2-(3-indolyl)ethyl (A8″-38); (4-phenyl)phenyl (A8″-39); n-nonyl (A8″-40); CH₃—OCH₂CH₂—OCH₂— (A8″-41) and CH₃—(OCH₂CH₂)₂—OCH₂— (A8″-42).

The building block —B3-CO— within templates (a3) designates Gly or a D-amino acid residue. Preferred values for B3 are:
D-Ala D-Alanine
D-Arg D-Arginine
D-Asn D-Asparagine
D-Cys D-Cysteine
D-Gln D-Glutamine
Gly Glycine
D-His D-Histidine
D-Ile D-Isoleucine
D-Leu D-Leucine
D-Lys D-Lysine
D-Met D-Methionine
D-Phe D-Phenylalanine
D-Ser D-Serine
D-Thr D-Threonine
D-Trp D-Tryptophan
D-Tyr D-Tyrosine
D-Val D-Valine
D-Cit D-Citrulline
D-Orn D-Ornithine
D-tBuA D-t-Butylalanine
D-Sar D-Sarcosine
D-t-BuG D-tert.-Butylglycine
D-4AmPhe D-para-Aminophenylalanine
D-3AmPhe D-meta-Aminophenylalanine
D-2AmPhe D-ortho-Aminophenylalanine
D-Phe(mC(NH₂)═NH) D-meta-Amidinophenylalanine
D-Phe(pC(NH₂)═NH) D-para-Amidinophenylalanine
D-Phe(mNHC(NH₂)═NH) D-meta-Guanidinophenylalanine
D-Phe(pNHC(NH₂)═NH) D-para-Guanidinophenylalanine
D-Phg D-Phenylglycine
D-Cha D-Cyclohexylalanine
D-C₄al D-3-Cyclobutylalanine
D-C₅al D-3-Cyclopentylalanine
D-Nle D-Norleucine
D-2-Nal D-2-Naphthylalanine
D-1-Nal D-1-Naphthylalanine
D-4Cl-Phe D-4-Chlorophenylalanine
D-3Cl-Phe D-3-Chlorophenylalanine
D-2Cl-Phe D-2-Chlorophenylalanine
D-3,4Cl₂-Phe D-3,4-Dichlorophenylalanine
D-4F-Phe D-4-Fluorophenylalanine
D-3F-Phe D-3-Fluorophenylalanine
D-2F-Phe D-2-Fluorophenylalanine
D-Thi D-β-2-Thienylalanine
D-Tza D-2-Thiazolylalanine
D-Mso D-Methionine sulfoxide
D-AcLys D-N-Acetyllysine
D-Dpr D-2,3-Diaminopropionic acid
D-A₂Bu D-2,4-Diaminobutyric acid
D-Dbu (2R,3S)-2,3-Diaminobutyric acid
D-Abu D-γ-Aminobutyric acid (GABA)
D-Aha D-ε-Aminohexanoic acid
D-Aib D-α-Aminoisobutyric acid
D-Y(Bzl) D-O-Benzyltyrosine
D-Bip D-Biphenylalanine
S(Bzl) L-O-Benzylserine
D-T(Bzl) D-O-Benzylthreonine
D-hCha D-Homo-cyclohexylalanine
D-hCys D-Homo-cysteine
D-hSer D-Homo-serine
D-hArg D-Homo-arginine
D-hPhe D-Homo-phenylalanine
D-Bpa D-4-Benzoylphenylalanine
D-OctG D-Octylglycine
D-MePhe D-N-Methylphenylalanine
D-MeNle D-N-Methylnorleucine
D-MeAla D-N-Methylalanine
D-MeIle D-N-Methylisoleucine
D-MeVal D-N-Methvaline
D-MeLeu D-N-Methylleucine

In a particular embodiment, the template is ^{D}Pro-^{L}Pro, ^{D}Pro-4Hyp2, ^{D}Pro-Oic, ^{D}Pro-4 Mp1, ^{D}Ser-^{L}Pro, ^{D}4Hyp2-^{L}Pro or ^{D}Glu-^{L}Pro. Instead of residues of ^{D}Pro and/or ^{L}Pro, the template can also contain certain substituted derivatives thereof with substitution patterns as shown in formulae A8′ and A8″, hereinabove.

The peptidic chain Z of the β-hairpin mimetics described herein is generally defined in terms of amino acid residues belonging to one of the following groups:
Group C —NR²⁰CH(R⁷²)CO—; “hydrophobic: small to medium-sized”
Group D —NR²⁰CH(R⁷³)CO—; “hydrophobic: large aromatic or heteroaromatic”
Group E —NR²⁰CH(R⁷⁴)CO—; “polar-cationic” and “urea-derived”
Group F —NR²⁰CH(R⁸⁴)CO—; “polar-non-charged or anionic”
Group H —NR²⁰—CH(CO—)—(CH₂)₄₋₇—CH(CO—)—NR²⁰—; —NR²⁰—CH(CO—)—(CH₂)ₚSS(CH₂)ₚ—CH(CO—)—NR²⁰—; —NR²⁰—CH(CO—)—(—(CH₂)ₚNR²⁰CO(CH₂)ₚ—CH(CO—)—NR²⁰—; and —NR²⁰—CH(CO—)—(—(CH₂)ₚNR²⁰CONR²⁰(CH₂)ₚ—CH(CO—)—NR²⁰—; “interstrand linkage”

Furthermore, the amino acid residues in positions P4 and P5 of chain Z can also be Gly.

Group C comprises amino acid residues with small to medium-sized hydrophobic side chain groups according to the above general definition for substituent R⁷². A hydrophobic residue refers to an amino acid side chain that is uncharged at physiological pH and that is repelled by aqueous solution. Furthermore these side chains generally do not contain hydrogen bond donor groups, such as (but not limited to) primary and secondary amides, primary and secondary amines and the corresponding protonated salts thereof, thiols, alcohols, phosphonates, phosphates, ureas or thioureas. However, they may contain hydrogen bond acceptor groups such as ethers, thioethers, esters, tertiary amides, alkyl- or aryl phosphonates and phosphates or tertiary amines. Genetically encoded small-to-medium-sized amino acids include alanine, isoleucine, leucine, methionine and valine.

Group D comprises amino acid residues with aromatic and heteroaromatic side chain groups according to the above general definition for substituent R⁷³. An aromatic amino acid residue refers to a hydrophobic amino acid having a side chain containing at least one ring having a conjugated π-electron system (aromatic group). In addition they may contain hydrogen bond donor groups such as (but not limited to) primary and secondary amides, primary and secondary amines and the corresponding protonated salts thereof, thiols, alcohols, phosphonates, phosphates, ureas or thioureas, and hydrogen bond acceptor groups such as (but not limited to) ethers, thioethers, esters, tetriary amides, alkyl- or aryl phosphonates and -phosphates, or tertiary amines. Genetically encoded aromatic amino acids include phenylalanine and tyrosine.

A heteroaromatic amino acid residue refers to a hydrophobic amino acid having a side chain containing at least one ring having a conjugated π-system incorporating at least one heteroatom such as (but not limited to) O, S and N according to the above general definition for substituent R⁷⁷. In addition such residues may contain hydrogen bond donor groups such as (but not limited to) primary and secondary amides, primary and secondary amines and the corresponding protonated salts thereof, thiols, alcohols, phosphonates, phosphates, ureas or thioureas, and hydrogen bond acceptor groups such as (but not limited to) ethers, thioethers, esters, tetriary amides, alkyl- or aryl phosphonates and phosphates or tertiary amines. Genetically encoded heteroaromatic amino acids include tryptophan and histidine.

Group E comprises amino acids containing side chains with polar-cationic, acylamino- and urea-derived residues according to the above general definition for substituent R⁷⁴. Polar-cationic refers to a basic side chain which is protonated at physiological pH. Genetically encoded polar-cationic amino acids include arginine, lysine and histidine. Citrulline is an example for an urea derived amino acid residue.

Group F comprises amino acids containing side chains with polar-non-charged or anionic residues according to the above general definition for substituent R⁸⁴. A polar-non-charged or anionic residue refers to a hydrophilic side chain that is uncharged and, respectively anionic at physiological pH (carboxylic acids being included), but that is not repelled by aqueous solutions. Such side chains typically contain hydrogen bond donor groups such as (but not limited to) primary and secondary amides, carboxyclic acids and esters, primary and secondary amines, thiols, alcohols, phosphonates, phosphates, ureas or thioureas. These groups can form hydrogen bond networks with water molecules. In addition they may also contain hydrogen bond acceptor groups such as (but not limited to) ethers, thioethers, esters, tetriary amides, carboxylic acids and carboxylates, alkyl- or aryl phosphonates and phosphates or tertiary amines. Genetically encoded polar-non-charged amino acids include asparagine, cysteine, glutamine, serine and threonine, but also aspartic acid and glutamic acid.

Group H comprises side chains of preferably (L)-amino acids at opposite positions of the β-strand region that can form an interstrand linkage. The most widely known linkage is the disulfide bridge formed by cysteines and homo-cysteines positioned at opposite positions of the β-strand. Various methods are known to form disulfide linkages including those described by: J. P. Tam et al. Synthesis 1979, 955-957; Stewart et al. Solid Phase Peptide Synthesis, 2d Ed., Pierce Chemical Company, III., 1984; Ahmed et al. J. Biol. Chem. 1975, 250, 8477-8482; and Pennington et al. Peptides, pages 164-166, Giralt and Andreu, Eds., ESCOM Leiden, The Netherlands, 1990. Most advantageously, for the scope of the present invention, disulfide linkages can be prepared using acetamidomethyl (Acm)-protective groups for cysteine. Another well established interstrand linkage consists in linking ornithines and lysines, respectively, with glutamic and aspartic acid residues located at opposite β-strand positions by means of an amide bond formation. Preferred protective groups for the side chain amino-groups of ornithine and lysine are allyloxycarbonyl (Alloc) and allylesters for aspartic and glutamic acid. Finally, interstrand linkages can also be established by linking the amino groups of lysine and ornithine located at opposite β-strand positions with reagents such as N,N-carbonylimidazole to form cyclic ureas.

As mentioned earlier, positions for interstrand linkages are positions P2 and P7; taken together. Such interstrand linkages are known to stabilize the β-hairpin conformations and thus constitute an important structural element for the design of β-hairpin mimetics.

Most preferred amino acid residues in chain Z are those derived from natural α-amino acids. Hereinafter follows a list of amino acids which, or the residues of which, are suitable for the purposes of the present invention, the abbreviations corresponding to generally adopted usual practice:

| three letter code | | one letter code |
|---|---|---|
| Ala | L-Alanine | A |
| ^{D}Ala | D-Alanine | ^{D}A |
| Arg | L-Arginine | R |
| Asn | L-Asparagine | N |
| Asp | L-Aspartic acid | D |
| Cys | L-Cysteine | C |
| Glu | L-Glutamic acid | E |
| Glu(cHx) | L-Glutamic acid cyclohexyl ester | |
| Gln | L-Glutamine | Q |
| Gly | Glycine | G |
| His | L-Histidine | H |
| Ile | L-Isoleucine | I |
| Leu | L-Leucine | L |
| Lys | L-Lysine | K |
| Met | L-Methionine | M |
| Phe | L-Phenylalanine | F |
| Pro | L-Proline | P |
| ^{D}Pro | D-Proline | ^{D}P |
| Ser | L-Serine | S |
| Thr | L-Threonine | T |
| Trp | L-Tryptophan | W |
| ^{D}Trp | D-Tryptophan | ^{D}W |
| Trp(6Cl) | 6-Chloro-L-Tryptophan | |
| Tyr | L-Tyrosine | Y |
| Val | L-Valine | V |

Other α-amino acids which, or the residues of which, are suitable for the purposes of the present invention include:
Cit L-Citrulline
Orn L-Ornithine
tBuA L-t-Butylalanine
Sar L-Sarcosine
Pen L-Penicillamine
t-BuG L-tert.-Butylglycine
4AmPhe L-para-Aminophenylalanine
3AmPhe L-meta-Aminophenylalanine
2AmPhe L-ortho-Aminophenylalanine
Phe(mC(NH₂)═NH) L-meta-Amidinophenylalanine
Phe(pC(NH₂)═NH) L-para-Amidinophenylalanine
Phe(mNHC(NH₂)═NH) L-meta-Guanidinophenylalanine
Phe(pNHC(NH₂)═NH) L-para-Guanidinophenylalanine
Phg L-Phenylglycine
Cha L-3-Cyclohexylalanine
C₄al L-3-Cyclobutylalanine
C₅al L-3-Cyclopentylalanine
Nle L-Norleucine
2-Nal L-2-Naphthylalanine
1-Nal L-1-Naphthylalanine
4Cl-Phe L-4-Chlorophenylalanine
3Cl-Phe L-3-Chlorophenylalanine
2C1-Phe L-2-Chlorophenylalanine
3,4Cl₂-Phe L-3,4-Dichlorophenylalanine
4F-Phe L-4-Fluorophenylalanine
3F-Phe L-3-Fluorophenylalanine
2F-Phe L-2-Fluorophenylalanine
Tic 1,2,3,4-Tetrahydroisoquinoline-3-carboxylic acid
Oic (2S,3aS, 7aS)-1-Octahydro-1H-indole-2-carboxylic acid
Thi L-β-2-Thienylalanine
Tza L-2-Thiazolylalanine
Mso L-Methionine sulfoxide
AcLys L-N-Acetyllysine
Dpr L-2,3-Diaminopropionic acid
A₂Bu L-2,4-Diaminobutyric acid
Dbu (S)-2,3-Diaminobutyric acid
Abu γ-Aminobutyric acid (GABA)
Aha L-ε-Aminohexanoic acid
Aib L-α-Aminoisobutyric acid
Y(Bzl) L-O-Benzyltyrosine
Bip L-(4-phenyl)phenylalanine
S(Bzl) L-O-Benzylserine
T(Bzl) L-O-Benzylthreonine
hCha L-Homo-cyclohexylalanine
hCys L-Homo-cysteine
hSer L-Homo-serine
hArg L-Homo-arginine
hPhe L-Homo-phenylalanine
Bpa L-4-Benzoylphenylalanine
4-AmPyrr1 (2S,4S)-4-Amino-pyrrolidine-L-carboxylic acid
4-AmPyrr2 (2S,4R)-4-Amino-pyrrolidine-L-carboxylic acid
4-PhePyrr1 (2S,5R)-4-Phenyl-pyrrolidine-L-carboxylic acid
4-PhePyrr2 (2S,5S)-4-Phenyl-pyrrolidine-L-carboxylic acid
5-PhePyrr1 (2S,5R)-5-Phenyl-pyrrolidine-L-carboxylic acid
5-PhePyrr2 (2S,5S)-5-Phenyl-pyrrolidine-L-carboxylic acid
4Hyp1 (4S)-L-Hydroxyproline
4Hyp2 (4R)-L-Hydroxyproline
4 Mp1 (4S)-L-Mercaptoproline
4 Mp2 (4R)-L-Mercaptoproline
Pip L-Pipecolic acid
^{D}Pip D-Pipecolic acid
OctG L-Octylglycine
NGly N-Methylglycine
MePhe L-N-Methylphenylalanine
MeNle L-N-Methylnorleucine
MeAla L-N-Methylalanine
MeIle L-N-Methylisoleucine
MeVal L-N-Methylvaline
MeLeu L-N-Methylleucine

Particularly preferred residues for group C are:
Ala L-Alanine
D-Ala D-Alanine
Ile L-Isoleucine
Leu L-Leucine
Met L-Methionine
Val L-Valine
tBuA L-t-Butylalanine
t-BuG L-tert.-Butylglycine
Cha L-Cyclohexylalanine
C₄al L-3-Cyclobutylalanine
C₅al L-3-Cyclopentylalanine
Nle L-Norleucine
hCha L-Homo-cyclohexylalanine
OctG L-Octylglycine
MePhe L-N-Methylphenylalanine
MeNle L-N-Methylnorleucine
MeAla L-N-Methylalanine
MeIle L-N-Methylisoleucine
MeVal L-N-Methylvaline
MeLeu L-N-Methylleucine

Particularly preferred residues for group D are:
His L-Histidine
Phe L-Phenylalanine
Trp L-Tryptophan
Trp(6Cl) 6-Chloro-L-Tryptophan
Tyr L-Tyrosine
Phg L-Phenylglycine
2-Nal L-2-Naphthylalanine
1-Nal L-1-Naphthylalanine
4Cl-Phe L-4-Chlorophenylalanine
3Cl-Phe L-3-Chlorophenylalanine
2Cl-Phe L-2-Chlorophenylalanine
3,4Cl₂-Phe L-3,4-Dichlorophenylalanine
4F-Phe L-4-Fluorophenylalanine
3F-Phe L-3-Fluorophenylalanine
2F-Phe L-2-Fluorophenylalanine
Thi L-β-2-Thienylalanine
Tza L-2-Thiazolylalanine
Y(Bzl) L-O-Benzyltyrosine
Bip L-Biphenylalanine
S(Bzl) L-O-Benzylserine
T(Bzl) L-O-Benzylthreonine
hPhe L-Homo-phenylalanine
Bpa L-4-Benzoylphenylalanine
PirrAla L-2-(3′-pyrrolidinyl)-alanine
NMePhe L-N-Methylphenylalanine
4-PyrAla L-2-(4′-Pyridyl)-alanine

Particularly preferred residues for group E are
Arg L-Arginine
Lys L-Lysine
Orn L-Ornithine
Dpr L-2,3-Diaminopropionic acid
A₂Bu L-2,4-Diaminobutyric acid
Dbu (2S,3S)-2,3-Diaminobutyric acid
Phe(pNH₂) L-para-Aminophenylalanine
Phe(mNH₂) L-meta-Aminophenylalanine
Phe(oNH₂) L-ortho-Aminophenylalanine
hArg L-Homo-arginine
Phe(mC(NH₂)═NH) L-meta-Amidinophenylalanine
Phe(pC(NH₂)═NH) L-para-Amidinophenylalanine
Phe(mNHC(NH₂)═NH) L-meta-Guanidinophenylalanine
Phe(pNHC(NH₂)═NH) L-para-Guanidinophenylalanine
DimK L-(N′,N′-Dimethyl)-lysine
Isorn L-(N′,N′-diisobutyl)-ornithine
NMeR L-N-Methylarginine
NMeK L-N-Methyllysine

Particularly preferred residues for group F are
Asn L-Asparagine
Asp L-Aspartic acid
Cys L-Cysteine
Gln L-Glutamine
Glu L-Glutamic acid
Glu(cHx) L-Glutamic acid cyclohexyl ester
Ser L-Serine
Thr L-Threonine
Cit L-Citrulline
Pen L-Penicillamine
AcLys L-N^{ε}-Acetyllysine
hCys L-Homo-cysteine
hSer L-Homo-serine

Generally, the peptidic chain Z within the β-hairpin mimetics of the invention comprises 8 amino acid residues. The positions P1 to P8 of each amino acid residue in the chain Z are unequivocally defined as follows: P1 represents the first amino acid in the chain Z that is coupled with its N-terminus to the C-terminus of the templates (b)-(p), or of group —B—CO— in template (a1), or of group -A-CO— in template (a2), or of group —B—CO— in template (a3); and P8 represents the last amino acid in the chain Z that is coupled with its C-terminus to the N-terminus of the templates (b)-(p), or of group -A-CO— in template (a1), or of group —B—CO— in template (a2), or of group —B3-CO— in template (a3). Each of the positions P1 to P8 will contain an amino acid residue belonging to one of the above types C D, E, F, H, or being Gly or Pro, as defined above.

The α-amino acid residues in positions 1 to 8 of the chain Z are preferably:
P1: of type C, or of type D, or of type F;
P2: of type C, or of type F;
P3: or of type C, or of type D;
P4: of type C, or of type D, or of type F, or the residue is Gly;
P5: of type C, or of type D, or of type E, or of type F, or the residue is Gly;
P6: of type C, or of type D;
P7: of type C, or of type D, or of type F;
P8: of type C, or of type D, or of type F; or P2 and P7, taken together, form a group of type H;
at P4 and P5 also D-isomers being possible.

Most preferably the α-amino acid residues in positions 1 to 8 are:
P1: Phe, Glu, Cha, Met, Asp;
P2: Glu, Thr, Ala, Leu, Cys;
P3: Trp(6Cl), Trp, Ala, Phe, Tyr;
P4: Leu, Gly, Tyr, Cys, Trp, ^{D}Trp;
P5: Ala, ^{D}Ala, Gly, Tyr, Asp, Lys, Orn;
P6: Trp, OctG, Ala, Tyr;
P7: Glu, Ala, Tyr, Leu, Cys; and
P8: Phe, Trp, Glu(cHx), Ile, Met, Glu, Cha, Leu, Val; and
Cys if present at P2 and P7 can form a disulfide bridge.

For β-peptidomimetics having an agonizing or antagonizing activity against urotensin II the α-amino acid residues in positions 1 to 8 of the chain Z are preferably:
P1: of type F;
P2: of type F;
P3: of type D;
P4: of type D;
P5: of type E;
P6: of type D;
P7: of type F;
P8: of type C; or
P2 and P7, taken together, form a group of type H;
at P4 D-isomers being possible;
   most preferably:
P1: Asp;
P2: Cys;
P3: Phe, Tyr;
P4: Trp, ^{D}Trp;
P5: Lys, Orn;
P6: Tyr;
P7: Cys,
P8: Cha, Leu, Val; and
Cys at P2 and P7 may form a disulfide bridge.

For inhibitors of the STAT6/NCoA-1 interaction the α-amino acid residues in positions 1 to 8 of the chain Z are preferably:
P1: of type C, or of type D, or of type F;
P2: of type C, or of type F;
P3: or of type C, of type D;
P4: of type C, or of type D, or of type F, or the residue is Gly;
P5: of type C, or of type D, or of type F, or the residue is Gly;
P6: of type C, or of type D;
P7: of type C, or of type D, or of type F;
P8: of type C, or of type D, or of type F; or
P2 and P7, taken together, form a group of type H;
at P5 also D-isomers being possible;
   most preferably:
P1: Phe, Glu, Cha, Met;
P2: Glu, Thr, Ala, Leu;
P3: Trp(6Cl), Trp, Ala;
P4: Leu, Gly, Tyr, Cys;
P5: Ala, ^{D}Ala, Gly, Tyr, Asp;
P6: Trp, OctG, Ala;
P7: Glu, Ala, Tyr, Leu;
P8: Phe, Trp, Glu(cHx), Ile, Met, Glu, Cha;

Particularly preferred β-peptidomimetics of the invention include those described in Examples 1, 2, 9, 19, 31 and 32.

The processes of the invention can advantageously be carried out as parallel array syntheses to yield libraries of template-fixed β-hairpin peptidomimetics of the above general formula I. Such parallel syntheses allow one to obtain arrays of numerous (normally 12 to 192, typically 96) compounds of general formula I in high yields and defined purities, minimizing the formation of dimeric and polymeric by-products. The proper choice of the functionalized solid-support (i.e. solid support plus linker molecule), templates and site of cyclization play thereby key roles.

The functionalized solid support is conveniently derived from polystyrene crosslinked with, preferably 1-5%, divinylbenzene; polystyrene coated with polyethyleneglycol spacers (Tentagel®); and polyacrylamide resins (see also D. Obrecht, J.-M. Villalgordo, “Solid-Supported Combinatorial and Parallel Synthesis of Small-Molecular-Weight Compound Libraries”, Tetrahedron Organic Chemistry Series, Vol. 17, Pergamon, Elsevier Science, 1998).

The solid support is functionalized by means of a linker, i.e. a bifunctional spacer molecule which contains on one end an anchoring group for attachment to the solid support and on the other end a selectively cleavable functional group used for the subsequent chemical transformations and cleavage procedures. For the purposes of the present invention two types of linkers can be used:

Type 1 linkers are designed to release the amide group under acid conditions (H. Rink, Tetrahedron Lett. 1987, 28, 3783-3790). Linkers of this kind form amides of the carboxyl group of the amino acids; examples of resins functionalized by such linker structures include 4-[(((2,4-dimethoxyphenyl)Fmoc-aminomethyl)phenoxyacetamido) aminomethyl] PS resin, 4-[(((2,4-dimethoxyphenyl)Fmoc-aminomethyl)phenoxyacetamido) aminomethyl]-4-methylbenzydrylamine PS resin (Rink amide MBHA PS Resin), and 4-[(((2,4-dimethoxyphenyl)Fmoc-aminomethyl)phenoxyacetamido) aminomethyl] benzhydrylamine PS-resin (Rink amide BHA PS resin). Preferably, the support is derived from polystyrene crosslinked with, most preferably 1-5%, divinylbenzene and functionalized by means of the 4-(((2,4-dimethoxyphenyl)Fmoc-aminomethyl)phenoxyacetamido) linker

Type 2 linkers are designed to eventually release the carboxyl group under acidic conditions. Linkers of this kind form acid-labile esters with the carboxyl group of the amino acids, usually acid-labile benzyl, benzhydryl and trityl esters; examples of such linker structures include 2-methoxy-4-hydroxymethylphenoxy (Sasrin® linker), 4-(2,4-dimethoxyphenyl-hydroxymethyl)-phenoxy (Rink linker), 4-(4-hydroxymethyl-3-methoxyphenoxy)butyric acid (HMPB linker), trityl and 2-chlorotrityl. Preferably, the support is derived from polystyrene crosslinked with, most preferably 1-5%, divinylbenzene and functionalized by means of the 2-chlorotrityl linker.

When carried out as parallel array syntheses the processes of the invention can be advantageously carried out as described herein below but it will be immediately apparent to those skilled in the art how these procedures will have to be modified in case it is desired to synthesize one single compound of the above formula I.

A number of reaction vessels (normally 12 to 192, typically 96) equal to the total number of compounds to be synthesized by the parallel method are loaded with 25 to 1000 mg, preferably 60 mg, of the appropriate functionalized solid support, preferably 1 to 3% cross-linked polystyrene or Tentagel resin.

The solvent to be used must be capable of swelling the resin and includes, but is not limited to, dichloromethane (DCM), dimethylformamide (DMF), N-methylpyrrolidone (NMP), dioxane, toluene, tetrahydrofuran (THF), ethanol (EtOH), trifluoroethanol (TFE), isopropylalcohol and the like. Solvent mixtures containing as at least one component a polar solvent (e.g. 20% TFE/DCM, 35% THF/NMP) are beneficial for ensuring high reactivity and solvation of the resin-bound peptide chains (G. B. Fields, C. G. Fields, J. Am. Chem. Soc. 1991, 113, 4202-4207).

With the development of various linkers that release the C-terminal carboxylic acid group under mild acidic conditions, not affecting acid-labile groups protecting functional groups in the side chain(s), considerable progresses have been made in the synthesis of protected peptide fragments. The 2-methoxy-4-hydroxybenzylalcohol-derived linker (Sasrin® linker, Mergler et al. Tetrahedron Lett. 1988, 29 4005-4008) is cleavable with diluted trifluoroacetic acid (0.5-1% TFA in DCM) and is stable to Fmoc deprotection conditions during the peptide synthesis, Boc/tBu-based additional protecting groups being compatible with this protection scheme. Other linkers which are suitable for the process of the invention include the super acid labile 4-(2,4-dimethoxyphenyl-hydroxymethyl)-phenoxy linker (Rink linker, H. Rink, Tetrahedron Lett. 1987, 28, 3787-3790), where the removal of the peptide requires 10% acetic acid in DCM or 0.2% trifluoroacetic acid in DCM; the 4-(4-hydroxymethyl-3-methoxyphenoxy)butyric acid-derived linker (HMPB-linker, Flörsheimer & Riniker, Peptides 1991, 1990 131) which is also cleaved with 1% TFA/DCM in order to yield a peptide fragment containing all acid labile side-chain protective groups; and, in particular, the 2-chlorotritylchloride linker (Barlos et al. Tetrahedron Lett. 1989, 30, 3943-3946), which allows the peptide detachment using a mixture of glacial acetic acid/trifluoroethanol/DCM (1:2:7) for 30 min

Suitable protecting groups for amino acids and, respectively, for their residues are, for example,
for the amino group (as is present e.g. also in the side-chain of lysine)
Cbz benzyloxycarbonyl
Boc tert.-butyloxycarbonyl
Fmoc 9-fluorenylmethoxycarbonyl
Alloc allyloxycarbonyl
Teoc trimethylsilylethoxycarbonyl
Tcc trichloroethoxycarbonyl
Nps o-nitrophenylsulfonyl;
Trt triphenymethyl or trityl;
for the carboxyl group (as is present e.g. also in the side-chain of aspartic and glutamic
acid) by conversion into esters with the alcohol components
tBu tert.-butyl
Bn benzyl
Me methyl
Ph phenyl
Pac Phenacyl
   Allyl
Tse trimethylsilylethyl
Tce trichloroethyl;
for the guanidino group (as is present e.g. in the side-chain of arginine)
Pmc 2,2,5,7,8-pentamethylchroman-6-sulfonyl;
Ts tosyl (i.e. p-toluenesulfonyl);
Cbz benzyloxycarbonyl;
Pbf pentamethyldihydrobenzofuran-5-sulfonyl;
for the hydroxy group (as is present e.g. in the side-chain of threonine and serine)
tBu tert.-butyl;
Bn benzyl;
Trt trityl;
and for the mercapto group (as is present e.g. in the side-chain of cysteine)
Acm acetamidomethyl;
tBu tert.-butyl;
Bn benzyl;
Trt trityl; and
Mtr 4-methoxytrityl.

The 9-fluorenylmethoxycarbonyl-(Fmoc)-protected amino acid derivatives are preferably used as the building blocks for the construction of the template-fixed β-hairpin loop mimetics of formula I. For the deprotection, i.e. cleaving off of the Fmoc group, 20% piperidine in DMF or 2% DBU (1,8-Diazabicyclo[5.4.0]undec-7-ene)/2% piperidine in DMF can be used.

The quantity of the reactant, i.e. of the amino acid derivative, is usually 1 to 20 equivalents based on the milliequivalents per gram (meq/g) loading of the functionalized solid support (typically 0.1 to 2.85 meq/g for polystyrene resins) originally weighed into the reaction tube. Additional equivalents of reactants can be used, if required, to drive the reaction to completion in a reasonable time. The preferred workstations (without, however, being limited thereto) are Labsource's Combi-chem station, Protein Technologies' Symphony and MultiSyn Tech's-Syro synthesizer, the latter additionally equipped with a transfer unit and a reservoir box during the process of detachment of the fully protected linear peptide from the solid support. All synthesizers are able to provide a controlled environment; for example, reactions can be accomplished at temperatures different from room temperature as well as under inert gas atmosphere, if desired.

Amide bond formation requires the activation of the α-carboxyl group for the acylation step. When this activation is being carried out by means of the commonly used carbodiimides such as dicyclohexylcarbodiimide (DCC, Sheehan & Hess, J. Am. Chem. Soc. 1955, 77, 1067-1068) or diisopropylcarbodiimide (DIC, Sarantakis et al Biochem. Biophys. Res. Commun. 1976, 73, 336-342), the resulting dicyclohexylurea and, respectively, diisopropylurea is insoluble and, respectively, soluble in the solvents generally used. In a variation of the carbodiimide method 1-hydroxybenzotriazole (HOBt, König & Geiger, Chem. Ber 1970, 103, 788-798) is included as an additive to the coupling mixture. HOBt prevents dehydration, suppresses racemization of the activated amino acids and acts as a catalyst to improve the sluggish coupling reactions. Certain phosphonium reagents have been used as direct coupling reagents, such as benzotriazol-1-yl-oxy-tris-(dimethylamino)-phosphonium hexafluorophosphate (BOP, Castro et al. Tetrahedron Lett. 1975, 14, 1219-1222; Synthesis 1976, 751-752), or benzotriazol-1-yl-oxy-tri s-pyrrolidino-phosphonium hexafluorophoshate (Py-BOP, Coste et al. Tetrahedron Lett. 1990, 31, 205-208), or 2-(1H-benzotriazol-1-yl-)1,1,3,3-tetramethyluronium tetrafluoroborate (TBTU), or hexafluorophosphate (HBTU, Knorr et al. Tetrahedron Lett. 1989, 30, 1927-1930); these phosphonium reagents are also suitable for in situ formation of HOBt esters with the protected amino acid derivatives. More recently diphenoxyphosphoryl azide (DPPA) or O-(7-aza-benzotriazol-1-yl)-N,N,N′,N′-tetramethyluronium tetrafluoroborate (TATU) or O-(7-aza-benzotriazol-1-yl)-N,N,N′,N′-tetramethyluronium hexafluorophosphate (HATU)/7-aza-1-hydroxy benzotriazole (HOAt, Carpino et al. Tetrahedron Lett. 1994, 35, 2279-2281) or —(6-chloro-1H-benzotriazol-1-yl-)-N,N,N′,N′-1,1,3,3-tetramethyluronium tetrafluoroborate (TCTU), or hexafluorophosphate (HCTU, Marder, Shivo and Albericio: HCTU and TCTU: New Coupling Reagents: Development and Industrial Applications, Poster Presentation, Gordon Conference February 2002) have also been used as coupling reagents.

Due to the fact that near-quantitative coupling reactions are essential, it is desirable to have experimental evidence for completion of the reactions. The ninhydrin test (Kaiser et al. Anal. Biochemistry 1970, 34, 595), where a positive colorimetric response to an aliquot of resin-bound peptide indicates qualitatively the presence of the primary amine, can easily and quickly be performed after each coupling step. Fmoc chemistry allows the spectrophotometric detection of the Fmoc chromophore when it is released with the base (Meienhofer et al. Int. J. Peptide Protein Res. 1979, 13, 35-42).

The resin-bound intermediate within each reaction vessel is washed free of excess of retained reagents, of solvents, and of by-products by repetitive exposure to pure solvent(s) by one of the two following methods:

1) The reaction vessels are filled with solvent (preferably 5 ml), agitated for 5 to 300 minutes, preferably 15 minutes, and drained to expel the solvent;
2) The reaction vessels are filled with solvent (preferably 5 ml) and drained into a receiving vessel such as a test tube or vial.
Both of the above washing procedures are repeated up to about 50 times (preferably about 10 times), monitoring the efficiency of reagent, solvent, and by-product removal by methods such as TLC, GC, or inspection of the washings.

The above described procedure of reacting the resin-bound compound with reagents within the reaction tubes followed by removal of excess reagents, by-products, and solvents is repeated with each successive transformation until the final resin-bound fully protected linear peptide has been obtained.

Before this fully protected linear peptide is detached from the solid support, it is possible, if desired, to selectively deprotect one or several protected functional group(s) present in the molecule and to appropriately substitute the reactive group(s) thus liberated. To this effect, the functional group(s) in question must initially be protected by a protecting group which can be selectively removed without affecting the remaining protecting groups present. Alloc (allyloxycarbonyl) is an example for such an amino protecting group which can be selectively removed, e.g. by means of Pd⁰ and phenylsilane in CH₂Cl₂, without affecting the remaining protecting groups, such as Fmoc, present in the molecule. The reactive group thus liberated can then be treated with an agent suitable for introducing the desired substituent. Thus, for example, an amino group can be acylated by means of an acylating agent corresponding to the acyl substituent to be introduced.

Before this fully protected linear peptide is detached from the solid support, it is also possible, if desired, to form an interstrand linkage between side-chains of appropriate amino acid residues at positions 2 and 7.

Interstrand linkages and their formation have been discussed above, in connection with the explanations made regarding groups of the type H which can, for example, be disulfide bridges formed by cysteine and homocysteine residues at positions 2 and 7; or lactam bridges formed by glutamic and aspartic acid residues linking ornithine and, respectively, lysine residues, or by glutamic acid residues linking 2,4-diaminobutyric acid residues located at positions 2 and 7 by amide bond formation. The formation of such interstrand linkages can be effected by methods well known in the art.

For the formation of disulfide bridges preferably a solution of 10 equivalents of iodine solution is applied in DMF or in a mixture of CH₂Cl₂/MeOH for 1.5 h which is repeated for another 3 h with a fresh iodine solution after filtering of the iodine solution, or in a mixture of DMSO and acetic acid solution, buffered with 5% with NaHCO₃ to pH 5-6 for 4 h, or in water after adjusted to pH 8 with ammonium hydroxide solution by stirring for 24 h, or in a solution of NMP and tri-n-butylphosphine (preferably 50 eq.).

For the formation of lactam bridges preferably a solution of 2 equivalents of HATU (N-[(dimethylamino)-1H-1,2,3-triazolo[4,5-b]pyridin-1-ylmethylene]-N-methyl-methanaminium hexafluorophosphate N-oxide) in dry DMF and a solution of 4 equivalents of DIPEA (Diisopropyl ethaylamine) in dry DMF is applied for 16 h.

Detachment of the fully protected linear peptide from the solid support is achieved by exposing the loaded resin with a solution of the cleavage reagent (preferably 3 to 5 ml). Temperature control, agitation, and reaction monitoring are implemented as described above. Via a transfer-unit the reaction vessels are connected with a reservoir box containing reservoir tubes to efficiently collect the cleaved product solutions. The resins remaining in the reaction vessels are then washed 2 to 5 times as above with 3 to 5 ml of an appropriate solvent to extract (wash out) as much of the detached products as possible. The product solutions thus obtained are combined, taking care to avoid cross-mixing. The individual solutions/extracts are then manipulated as needed to isolate the final compounds. Typical manipulations include, but are not limited to, evaporation, concentration, liquid/liquid extraction, acidification, basification, neutralization or additional reactions in solution.

The solutions containing fully protected linear peptide derivatives which have been cleaved off from the solid support and neutralized with a base, are evaporated. Cyclization is then effected in solution using solvents such as DCM, DMF, dioxane, THF and the like. Various coupling reagents which were mentioned earlier can be used for the cyclization. The duration of the cyclization is about 6-48 hours, preferably about 16 hours. The progress of the reaction is followed, e.g. by RP-HPLC (Reverse Phase High Performance Liquid Chromatography). Then the solvent is removed by evaporation, the fully protected cyclic peptide derivative is dissolved in a solvent which is not miscible with water, such as DCM, and the solution is extracted with water or a mixture of water-miscible solvents, in order to remove any excess of the coupling reagent.

Alternatively, the detachment and complete deprotection of the fully protected peptide from the solid support can be achieved manually in glass vessels.

Finally, the fully protected peptide derivative is treated with 95% TFA, 2.5% H₂O, 2.5% TIS or another combination of scavengers for effecting the cleavage of protecting groups. The cleavage reaction time is commonly 30 minutes to 12 hours, preferably about 2.5 hours.

After fully deprotection one of the following methods can be used for further work-up:

1) The volatiles are evaporated to dryness and the crude peptide is dissolved in 20% AcOH in water and extracted with isopropyl ether or other solvents which are suitable therefore. The aqueous layer is collected and evaporated to dryness, and the fully deprotected cyclic peptide derivative of formula I is obtained as end-product;
2) The fully deprotection mixture is concentrated under vacuum. Following precipitation of the fully deprotected peptide in diethylether at preferably 0° C. the solid is washed up to about 10 times, preferably 3 times, dried, and the fully deprotected cyclic peptide derivative of formula I is obtained as end-product.

As mentioned earlier, it is thereafter possible, if desired, to convert a fully deprotected product of formula I thus obtained into a pharmaceutically acceptable salt or to convert a pharmaceutically acceptable, or unacceptable, salt thus obtained into the corresponding free compound of formula I or into a different, pharmaceutically acceptable, salt. Any of these operations can be carried out by methods well known in the art.

The template starting materials of formula II used in the processes of the invention, pre-starting materials therefore, and the preparation of these starting and pre-starting materials are described in International Application PCT/EP02/01711 of the same applicants, published as WO 02/070547 A1.

The β-hairpin peptidomimetics of the invention can be used in a wide range of applications in order to treat, in particular (but not limited thereto), renal diseases, cardiorenal diseases, diabetes, inflammation, heart failure, hypertension, endothelial dysfunction, insulin resistance, hyperglycemia, allergic reactions including asthma and atopic diseases.

These β-hairpin peptidomimetics may be administered per se or may be applied as an appropriate formulation together with carriers, diluents or excipients well known in the art.

They can be administered singly, as mixtures of several of these β-hairpin peptidomimetics or in combination with other pharmaceutically active agents such as anti-inflammatory agents or antimicrobial agents or anti cancer agents or anti-HIV agents.

Pharmaceutical compositions comprising β-hairpin peptidomimetics of the invention may be manufactured by means of conventional mixing, dissolving, granulating, coated tablet-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active β-hairpin peptidomimetics into preparations which can be used pharmaceutically. Proper formulation depends upon the method of administration chosen.

For topical administration the β-hairpin peptidomimetics of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injections, the β-hairpin peptidomimetics of the invention may be formulated in adequate solutions, preferably in physiologically compatible buffers such as Hink's solution, Ringer's solution, or physiological saline buffer. The solutions may contain formulatory agents such as suspending, stabilizing and/or dispersing agents. Alternatively, the β-hairpin peptidomimetics of the invention may be in powder form for combination with a suitable vehicle, e.g., sterile pyrogen-free water, before use.

For transmucosal administration, penetrants appropriate to the barrier to be permeated are used in the formulation as known in the art.

For oral administration, the compounds can be readily formulated by combining the active β-hairpin peptidomimetics of the invention with pharmaceutically acceptable carriers well known in the art. Such carriers enable the β-hairpin peptidomimetics of the invention to be formulated as tablets, pills, dragees, capsules, liquids, gels, syrups, slurries, suspensions etc., for oral ingestion by a patient to be treated. For oral formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP); granulating agents; and binding agents. If desired, disintegrating agents may be added, such as cross-linked polyvinylpyrrolidones, agar, or alginic acid or a salt thereof, such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. In addition, flavoring agents, preservatives, coloring agents and the like may be added.

For buccal administration, the composition may take the form of tablets, lozenges, etc. formulated as usual.

For administration by inhalation, the β-hairpin peptidomimetics of the invention are conveniently delivered in form of an aeorosol spray from pressurized packs or a nebulizer, with the use of a suitable propellant, e.g. dichlorodifluoromethane, trichlorofluoromethane, carbon dioxide or another suitable gas. In the case of a pressurized aerosol the dose unit may be determined by providing a valve to deliver a metered amount. Capsules and cartridges of e.g. gelatin for use in an inhaler or insufflator may be formulated containing a powder mix of the β-hairpin peptidomimetics of the invention and a suitable powder base such as lactose or starch.

The compounds may also be formulated in rectal or vaginal compositions such as suppositories together with appropriate suppository bases such as cocoa butter or other glycerides.

In addition to the formulations described above, the β-hairpin peptidomimetics of the invention may also be formulated as depot preparations. Such long acting formulations may be administered by implantation (e.g. subcutaneously or intramuscularly) or by intramuscular injection. For the manufacture of such depot preparations the β-hairpin peptidomimetics of the invention may be formulated with suitable polymeric or hydrophobic materials (e.g. as an emulsion in an acceptable oil) or ion exchange resins, or as sparingly soluble salts.

In addition, other pharmaceutical delivery systems may be employed such as liposomes and emulsions as well known in the art. Certain organic solvents such as dimethylsulfoxide may also be employed. Additionally, the β-hairpin peptidomimetics of the invention may be delivered using a sustained-release system, such as semipermeable matrices of solid polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic agent, additional strategies for protein stabilization may be employed.

As the β-hairpin pepdidomimetics of the invention may contain charged residues, they may be included in any of the above-described formulations as such or as pharmaceutically acceptable salts. Pharmaceutically acceptable salts tend to be more soluble in aqueous and other protic solvents than are the corresponding free forms.

The β-hairpin peptidomimetics of the invention, or compositions thereof, will generally be used in an amount effective to achieve the intended purpose. It is to be understood that the amount used will depend on a particular application.

For topical administration a therapeutically effective dose can be determined using, for example, the in vitro assays provided in the examples. An ordinary skilled expert will be able to determine therapeutically effective amounts without undue experimentation.

For systemic administration, a therapeutically effective dose can be estimated initially from in vitro assays. For example, a dose can be formulated in animal models to achieve a circulating β-hairpin peptidomimetic concentration range that includes the IC₅₀ as determined in the cell culture (i.e. the concentration of a test compound that is lethal to 50% of a cell culture). Such information can be used to more accurately determine useful doses in humans.

Initial dosages can also be determined from in vivo data, e.g. animal models, using techniques that are well known in the art. One having ordinary skill in the art could readily optimize administration to humans based on animal data.

Dosage amounts may be adjusted individually to provide plasma levels of the β-hairpin peptidomimetics of the invention which are sufficient to maintain the therapeutic effect. Therapeutically effective serum levels may be achieved by administering multiple doses each day.

In cases of local administration or selective uptake, the effective local concentration of the β-hairpin peptidomimetics of the invention may not be related to plasma concentration. One having the ordinary skill in the art will be able to optimize therapeutically effective local dosages without undue experimentation.

The amount of β-hairpin peptidomimetics administered will, of course, be dependent on the subject being treated, on the subject's weight, the severity of the affliction, the manner of administration and the judgement of the prescribing physician.

Normally, a therapeutically effective dose of the β-hairpin peptidomimetics described herein will provide therapeutic benefit without causing substantial toxicity.

Toxicity of the β-hairpin peptidomimetics of the invention can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in humans. The dosage of the β-hairpin peptidomimetics of the invention lies preferably within a range of circulating concentrations that include the effective dose with little or no toxicity. The dosage may vary within the range depending upon the dosage form employed and the route of administration utilized. The exact formulation, route of administration and dose can be chosen by the individual physician in view of the patient's condition (see, e.g. Fingl et al. 1975, in: The Pharmacological Basis of Therapeutics, Ch.1, p. 1).

The following Examples illustrate the present invention without, however, limiting its scope in any way.

### EXAMPLES

### 1. Peptide Synthesis

### Coupling of the First Protected Amino Acid Residue to the Resin

1 g (1.4 mMol) of 2-chlorotritylchloride resin (1.4 mMol/g; Barbs et al. Tetrahedron Lett. 1989, 30, 3943-3946) was filled into a dried flask. The resin was suspended in CH₂Cl₂ (5 ml) and allowed to swell at room temperature under constant shaking for 30 min A solution of 0.98 mMol (0.7 eq) of the first suitably protected amino acid residue (see below) in CH₂Cl₂ (5 ml) completed by 960 μl (4 eq) of diisopropylethylamine (DIEA) was added. After shaking the reaction mixture for 4 hours at 25° C. the resin was filtered and washed successively with CH₂Cl₂ (1×), DMF (1×) and CH₂Cl₂ (1×). A solution of CH₂Cl₂/MeOH/DIEA (17/2/1, 10 ml) was added to the resin and the suspension was shaken for 30 min After filtration the resin was washed in the following order with CH₂Cl₂ (1×), DMF (1×), CH₂Cl₂ (1×), MeOH (1×), CH₂Cl₂ (1×), MeOH (1×), CH₂Cl₂ (2×), Et₂O (2×) and dried under vacuum for 6 hours.

Loading was typically 0.6-0.7 mMol/g.

The following preloaded resins were prepared: Fmoc-ProO-chlorotritylresin, Fmoc-4Hyp2(tBu)O-chlorotritylresin, Fmoc-OicO-chlorotritylresin, and Fmoc-4 Mp1(Trt)O-chloro-tritylresin.

The synthesis was carried out employing a Syro-peptide synthesizer (MultiSynTech) using 24-96 reaction vessel. In each vessel 0.04 mMol of the above resin were placed and the resin was swollen in CH₂Cl₂ and DMF for 15 min, respectively. The following reaction cycles were programmed and carried out:

| Step | Reagent | Time |
|---|---|---|
| 1 | DMF, wash and swell | 2 × 1 min |
| 2 | 20% piperidine/DMF | 1 × 5 min, 1 × 15 min |
| 3 | DMF, wash | 5 × 1 min |
| 4a | 5 eq Fmoc amino acid/DMF + | 1 × 60 min |
| | 5 eq HCTU/DMF, 10 eq DIEA | |
| 5 | DMF, wash | 3 × 1 min |

Step 4a was repeated once.

If not indicated otherwise, the final coupling of an amino acid is followed by an Fmoc deprotection by applying steps 1-3 of the above described reaction cycle.

The following Fmoc-protected amino acid derivative had to be synthesized before its usage in the linear peptide synthesis described above.

### Synthesis of N-Fmoc-Protected L-6-Chlorotryptophan

(modified procedure following E. Atherton, R. Sheppard, Solid phase peptide synthesis. A practical approach, IRL Press, Oxford, 1989, page 49).

### Cyclization and Work Up of Backbone Cyclized Peptides

### Cleavage of the Fully Protected Peptide Fragment

After completion of the synthesis, the resin (0.04 mMol) was suspended in 1 ml (0.13 mMol, 3.4 eq) of 1% TFA in CH₂Cl₂ (v/v) for 3 minutes, filtered, and the filtrate was neutralized with 1 ml (0.58 mMol, 14.5 eq) of 10% DIEA in CH₂Cl₂ (v/v). This procedure was repeated three times to ensure completion of the cleavage. The filtrate was evaporated to dryness and a sample of the product was fully deprotected by using a cleavage mixture containing 95% trifluoroacetic acid (TFA), 2.5% water and 2.5% triisopropylsilane (TIS) to be analyzed by reverse phase-HPLC (column C₁₈) and ESI-MS to monitor the efficiency of the linear peptide synthesis.

### Cyclization of the Linear Peptide

The fully protected linear peptide (0.04 mMol) was dissolved in DMF (4 μMol/ml). Then 30.4 mg (0.08 mMol, 2 eq) of HATU, 10.9 mg (0.08 mMol, 2 eq) of HOAt and 28 μl (0.16 mMol, 4 eq) DIEA were added, and the mixture was vortexed at 25° C. for 16 hours and subsequently concentrated under high vacuum. The residue was partitioned between CH₂Cl₂ and H₂O/CH₃CN (90/10; v/v). The CH₂Cl₂ phase was evaporated to yield the fully protected cyclic peptide.

### Fully Deprotecting the Cyclic Peptide

The cyclic peptide obtained was dissolved in 3 ml of the cleavage mixture containing 82.5% trifluoroacetic acid (TFA), 5% water, 5% thioanisole, 5% phenol and 2.5% ethandithiole (EDT). The mixture was allowed to stand at 25° C. for 2.5 hours and thereafter concentrated under vacuum. After precipitation of the cyclic fully deprotected peptide in diethylether (Et₂O) at 0° C. the solid was washed twice with Et₂O and dried. Cyclic peptides without designed β-strand linkages were purified by reverse phase HPLC, cyclic peptides arranged for additional β-strand linkages were processed as described below.

### Formation of Disulfide β-Strand Linkage and Purification

After deprotection, the crude peptide was dissolved in 9 ml of 5% AcOH (buffered with NaHCO₃ to pH 5-6). 0.5 ml DMSO were added and the solution was shaken overnight. Following evaporation the residue was purified by preparative reverse phase HPLC.

### Analytical Method 1a:

Analytical HPLC retention times (RT, in minutes) were determined using an Acquity HPLC BEH C18 1.7 μm column with the following solvents A (H₂O/CH₃CN, 95/5 [v/v], +0.1% TFA) and B (CH₃CN+0.09% TFA) and the gradient: 0 min: 99% A, 1% B; 0.2 min: 99% A, 1% B; 4 min: 5% A, 95% B; 4.2 min: 5% A, 95% B; 4.25 min: 99% A, 1% B; 5.0 min: 99% A, 1% B.

### Analytical Method 1b:

Analytical HPLC retention times (RT, in minutes) were determined using an Acquity HPLC BEH C18 1.7 μm column with the following solvents A (H₂O+0.1% TFA) and B (CH₃CN+0.09% TFA) and the gradient: 0 min: 95% A, 5% B; 0.2 min: 95% A, 5% B; 4 min: 5% A, 95% B; 4.2 min: 5% A, 95% B; 4.25 min: 95% A, 5% B; 5.0 min: 95% A, 5% B.

### Analytical Method 2:

Analytical HPLC retention times (RT, in minutes) were determined using an Acquity HPLC BEH C18 1.7 μm column with the following solvents A (H₂O/CH₃CN, 95/5 [v/v], +0.1% TFA) and B (CH₃CN+0.09% TFA) and the gradient: 0 min: 99% A, 1% B; 0.2 min: 99% A, 1% B; 4 min: 35% A, 65% B; 4.05 min: 5% A, 95% B; 4.20 min: 5% A, 95% B; 4.25 min: 99% A, 1% B; 4.5 min: 99% A, 1% B.

### Analytical Method 3:

Analytical HPLC retention times (RT, in minutes) were determined using a zorbaxEclipsedXDBC18 column with the following solvents A (H₂O+0.1% TFA) and B (CH₃CN+0.1% TFA) and the gradient: 0 min: 60% A, 40% B; 21 min: 10% A, 90% B; 27 min. 100% B.

### Analytical Method 4:

Analytical HPLC retention times (RT, in minutes) were determined using a Laubscher Labs Interchrom 218QTP54 C18, 250×4.6 mm, 5 μm, 300 A with the following solvents A (H₂O+0.1% TFA) and B (CH₃CN+0.1% TFA) and the gradient: 0 min: 70% A, 30% B; 16.7 min: 100% B.

Examples 1, 4 and 6-27 are shown in Table 1. The peptides were synthesized starting with the amino acid Pro which was grafted to the resin. Starting resin was Fmoc-ProO—-chlorotrityl resin, which was prepared as described above. The linear peptide was synthesized on solid support according to the procedure described above in the following sequence: Resin-Pro-^{D}Pro-P8-P7-P6-P5-P4-P3-P2-P1. Following a final Fmoc deprotection as described above, the peptide was cleaved from the resin, cyclized, deprotected and purified as indicated above.

For Ex.4 HPLC-retention times (minutes) were determined using the gradient method 1b, for Ex.6, 9-10, 15-16 and 26-27 the gradient method 1a was applied; for Ex.7-8, 11-12 and 19-25 HPLC-retention times (minutes) were determined using the gradient method 3 and finally for Ex.1, 13-14 and 17-18 HPLC-retention times were identified by using method 4 as described above.

Example 2 is shown in Table 1. The peptide was synthesized starting with the amino acid Hyp which was grafted to the resin. Starting resin was Fmoc-4Hyp2(tBu)O-chlorotrityl resin, which was prepared as described above. The linear peptide was synthesized on solid support according to the procedure described above in the following sequence: Resin-Hyp-^{D}Pro-P8-P7-P6-P5-P4-P3-P2-P1. Following a final Fmoc deprotection as described above, the peptide was cleaved from the resin, cyclized, deprotected and purified as indicated above.

HPLC-retention times (minutes) was determined using the gradient method 1 as described above.

Example 3 is shown in Table 1. The peptide was synthesized starting with the amino acid Oic which was grafted to the resin. Starting resin was Fmoc-OicO-chlorotrityl resin, which was prepared as described above. The linear peptide was synthesized on solid support according to the procedure described above in the following sequence: Resin-Oic-^{D}Pro-P8-P7-P6-P5-P4-P3-P2-P1. Following a final Fmoc deprotection as described above, the peptide was cleaved from the resin, cyclized, deprotected and purified as indicated above. HPLC-retention time (minutes) was determined using the gradient method 1 as described above.

Example 5 is shown in Table 1. The peptide was synthesized starting with the amino acid Mp1 which was grafted to the resin. Starting resin was Fmoc-4 Mp1(Trt)O-chlorotrityl resin, which was prepared as described above. The linear peptide was synthesized on solid support according to the procedure described above in the following sequence: Resin-4 Mp1-^{D}Pro-P8-P7-P6-P5-P4-P3-P2-P1. Following a final Fmoc deprotection as described above, the peptide was cleaved from the resin, cyclized, deprotected and purified as indicated above.

HPLC-retention time (minutes) was determined using the gradient method 1 as described above

Example 28 is shown in Table 1. The peptide was synthesized starting with the amino acid Pro which was grafted to the resin. Starting resin was Fmoc-ProO-chlorotrityl resin, which was prepared as described above. The linear peptide was synthesized on solid support according to the procedure described above in the following sequence: Resin-Pro-^{D}Ser-P8-P7-P6-P5-P4-P3-P2-P1. Following a final Fmoc deprotection as described above, the peptide was cleaved from the resin, cyclized, deprotected and after formation of the disulfide n-strand linkage purified as indicated above.

HPLC-retention time (minutes) was determined using the gradient method 2 as described above

Example 29 is shown in Table 1. The peptide was synthesized starting with the amino acid Pro which was grafted to the resin. Starting resin was Fmoc-ProO-chlorotrityl resin, which was prepared as described above. The linear peptide was synthesized on solid support according to the procedure described above in the following sequence: Resin-Pro-^{D}Hyp-P8-P7-P6-P5-P4-P3-P2-P1. Following a final Fmoc deprotection as described above, the peptide was cleaved from the resin, cyclized, deprotected and after formation of the disulfide β-strand linkage purified as indicated above.

HPLC-retention time (minutes) was determined using the gradient method 2 as described above

Example 30 is shown in Table 1. The peptide was synthesized starting with the amino acid Pro which was grafted to the resin. Starting resin was Fmoc-ProO-chlorotrityl resin, which was prepared as described above. The linear peptide was synthesized on solid support according to the procedure described above in the following sequence: Resin-Pro-^{D}Glu-P8-P7-P6-P5-P4-P3-P2-P1. Following a final Fmoc deprotection as described above, the peptide was cleaved from the resin, cyclized, deprotected and after formation of the disulfide β-strand linkage purified as indicated above.

HPLC-retention time (minutes) was determined using the gradient method 2 as described above

Examples 31-35 are shown in Table 1. The peptides were synthesized starting with the amino acid Pro which was grafted to the resin. Starting resin was Fmoc-ProO-chlorotrityl resin, which was prepared as described above. The linear peptides were synthesized on solid support according to the procedure described above in the following sequence: Resin-Pro-^{D}Pro-P8-P7-P6-P5-P4-P3-P2-P1. Following a final Fmoc deprotection as described above, the peptides were cleaved from the resin, cyclized, deprotected and after formation of the disulfide β-strand linkage purified as indicated above.

HPLC-retention times (minutes) were determined using the gradient method 2 as described above

TABLE 1
Examples (Ex.)

| | | | | | | | | | | | Purity | | |
|---|---|---|---|---|---|---|---|---|---|---|---|---|---|
| Ex. | Seq ID | P1 | P2 | P3 | P4 | P5 | P6 | P7 | P8 | Template | %^{a)} | [M + H] | RT |
| 1 | SEQ ID NO: 1 | Phe | Glu | Trp(6Cl) | Leu | Ala | Trp | Glu | Phe | ^{D}Pro^{L}Pro | 95 | 1338.7 | 12.20 |
| 2 | SEQ ID NO: 2 | Phe | Glu | Trp | Leu | Ala | Trp | Glu | Phe | ^{D}Pro^{L}4Hyp2 | 90 | 1319.9 | 3.20 |
| 3 | SEQ ID NO: 3 | Phe | Glu | Trp | Leu | Ala | Trp | Glu | Trp | ^{D}Pro^{L}Oic | 95 | 1357.7 | 3.71 |
| 4 | SEQ ID NO: 4 | Phe | Glu | Trp | Leu | ^{D}A1a | Trp | Glu | Phe | ^{D}Pro^{L}Pro | 95 | 1303.7 | 3.49 |
| 5 | SEQ ID NO: 5 | Phe | Thr | Trp(6Cl) | Leu | Ala | Trp | Glu | Phe | ^{D}Pro^{L}4Mp1 | 85 | 1369.9 | 3.65 |
| 6 | SEQ ID NO: 6 | Phe | Glu | Trp(6Cl) | Leu | Ala | OctG | Glu | Phe | ^{D}Pro^{L}Pro | 90 | 1320.9 | 4.24 |
| 7 | SEQ ID NO: 7 | Phe | Glu | Trp | Gly | Ala | Trp | Glu | Phe | ^{D}Pro^{L}Pro | 95 | 1247.7 | 12.70 |
| 8 | SEQ ID NO: 8 | Phe | Glu | Trp | Leu | Gly | Trp | Glu | Phe | ^{D}Pro^{L}Pro | 95 | 1289.8 | 14.70 |
| 9 | SEQ ID NO: 9 | Phe | Glu | Trp | Leu | Ala | Trp | Glu | Glu(cHx) | ^{D}Pro^{L}Pro | 90 | 1402.0 | 3.82 |
| 10 | SEQ ID NO: 10 | Phe | Glu | Trp | Leu | Ala | Trp | Glu | Ile | ^{D}Pro^{L}Pro | 60 | 1269.7 | 3.48 |
| 11 | SEQ ID NO: 11 | Phe | Glu | Trp | Tyr | Ala | Trp | Glu | Phe | ^{D}Pro^{L}Pro | 95 | 1354.7 | 12.90 |
| 12 | SEQ ID NO: 12 | Phe | Glu | Trp | Leu | Tyr | Trp | Glu | Phe | ^{D}Pro^{L}Pro | 92 | 1396.8 | 15.10 |
| 13 | SEQ ID NO: 13 | Phe | Glu | Trp(6Cl) | Leu | Asp | Trp | Ala | Phe | ^{D}Pro^{L}Pro | 95 | 1323.5 | 11.60 |
| 14 | SEQ ID NO: 14 | Phe | Ala | Trp(6Cl) | Leu | Asp | Trp | Glu | Phe | ^{D}Pro^{L}Pro | 95 | 1325.6 | 12.10 |
| 15 | SEQ ID NO: 15 | Phe | Glu | Trp(6Cl) | Cys | Ala | Trp | Glu | Phe | ^{D}Pro^{L}Pro | 50 | 1327.6 | 3.48 |
| 16 | SEQ ID NO: 16 | Phe | Glu | Trp | Leu | Asp | Trp | Glu | Met | ^{D}Pro^{L}Pro | 80 | 1331.9 | 3.25 |
| 17 | SEQ ID NO: 17 | Phe | Glu | Ala | Leu | Asp | Trp | Glu | Phe | ^{D}Pro^{L}Pro | 95 | 1332.6 | 10.00 |
| 18 | SEQ ID NO: 18 | Phe | Glu | Trp(6Cl) | Leu | Asp | Ala | Glu | Phe | ^{D}Pro^{L}Pro | 95 | 1266.5 | 10.30 |
| 19 | SEQ ID NO: 19 | Phe | Glu | Trp | Leu | Asp | Trp | Glu | Phe | ^{D}Pro^{L}Pro | 95 | 1347.7 | 13.80 |
| 20 | SEQ ID NO: 20 | Phe | Glu | Trp | Leu | Ala | Trp | Glu | Glu | ^{D}Pro^{L}Pro | 95 | 1285.8 | 9.40 |
| 21 | SEQ ID NO: 21 | Phe | Glu | Trp | Leu | Ala | Trp | Tyr | Phe | ^{D}Pro^{L}Pro | 95 | 1337.9 | 16.00 |
| 22 | SEQ ID NO: 22 | Phe | Leu | Trp | Leu | Ala | Trp | Glu | Phe | ^{D}Pro^{L}Pro | 95 | 1287.7 | 16.20 |
| 23 | SEQ ID NO: 23 | Glu | Glu | Trp | Leu | Ala | Trp | Glu | Phe | ^{D}Pro^{L}Pro | 95 | 1285.7 | 9.30 |
| 24 | SEQ ID NO: 24 | Phe | Glu | Trp | Leu | Ala | Trp | Leu | Phe | ^{D}Pro^{L}Pro | 95 | 1287.9 | 16.70 |
| 25 | SEQ ID NO: 25 | Cha | Glu | Trp | Leu | Ala | Trp | Glu | Phe | ^{D}Pro^{L}Pro | 90 | 1309.8 | 17.20 |
| 26 | SEQ ID NO: 26 | Cha | Glu | Trp | Leu | Ala | Trp | Glu | Cha | ^{D}Pro^{L}Pro | 80 | 1315.4 | 4.00 |
| 27 | SEQ ID NO: 27 | Met | Glu | Trp | Leu | Asp | Trp | Glu | Phe | ^{D}Pro^{L}Pro | 80 | 1331.9 | 4.29 |
| 28 | SEQ ID NO: 28 | Asp | Cys | Phe | Trp | Lys | Tyr | Cys | Leu | ^{D}Ser^{L}Pro | 95 | 1243.0 | 3.12 |
| 29 | SEQ ID NO: 29 | Asp | Cys | Phe | Trp | Lys | Tyr | Cys | Leu | ^{D}4Hyp2^{L}Pro | 95 | 1267.8 | 3.05 |
| 30 | SEQ ID NO: 30 | Asp | Cys | Phe | Trp | Lys | Tyr | Cys | Leu | ^{D}Glu^{L}Pro | 90 | 1283.5 | 3.10 |
| 31 | SEQ ID NO: 31 | Asp | Cys | Phe | Trp | Lys | Tyr | Cys | Val | ^{D}Pro^{L}Pro | 90 | 1237.3 | 3.11 |
| 32 | SEQ ID NO: 32 | Asp | Cys | Phe | ^{D}Trp | Orn | Tyr | Cys | Val | ^{D}Pro^{L}Pro | 90 | 1223.4 | 3.06 |
| 33 | SEQ ID NO: 33 | Asp | Cys | Tyr | Trp | Lys | Tyr | Cys | Leu | ^{D}Pro^{L}Pro | 90 | 1267.5 | 2.95 |
| 34 | SEQ ID NO: 34 | Asp | Cys | Phe | ^{D}Trp | Lys | Tyr | Cys | Val | ^{D}Pro^{L}Pro | 85 | 1237.5 | 3.07 |
| 35 | SEQ ID NO: 35 | Asp | Cys | Phe | Trp | Lys | Tyr | Cys | Cha | ^{D}Pro^{L}Pro | 90 | 1292.8 | 3.60 |

Cys in pos.2 and 7 in Ex. 28-35 form a disulfide bridge,
^{a)}%-purity of compounds after prep. HPLC.

### 2. Biological Methods

### 2.1. Preparation of the Peptides

Lyophilized peptides were weighed on a Microbalance (Mettler MT5) and dissolved in sterile water to a final concentration of 1 mM unless stated otherwise. Stock solutions were kept at +4° C., light protected.

### 2.2. Cell Culture

Mouse pre-B cells were cultured in RPMI1640 plus 5% FBS, antibiotic/antimycotic, non essential amino acid, 50 μM β-mercaptoethanol and 1 mM natrium pyruvate. HELA cells were maintained in RPMI1640 plus 10% FBS, pen/strept and 2 mM L-glutamine. Cos-7 cells were grown in DMEM medium with 4500 mg/mL glucose supplemented with 10% FCS, pen/strept and 2 mM L-glutamine. All cell lines were grown at 37° C. at 5% CO₂. Cell media, media supplements, PBS-buffer, HEPES, antibiotic/antimycotic, pen/strept, non essential amino acid, L-glutamine, β-mercaptoethanol and sera were purchased from Gibco (Pailsey, UK). All fine chemicals were supplied by Merck (Darmstadt, Germany).

### 2.3. Ca²⁺⁻Assay: UTR2 Receptor-Agonizing and Antagonizing Activity of the Peptides

The mouse pre-B cell line 300-19 was stably transfected with the cDNA encoding the human UTR2 receptor (GenBank Acc# NM_{—}018949), and expression was confirmed with a positive calcium signal in response to human urotensin (Sigma Aldrich). Increases in intracellular calcium were monitored using a Flexstation 384 (Molecular Devices, Sunnyvale, Calif.). The cells were batch loaded with the Calcium 4 Assay kit (Molecular Devices) in assay buffer (Hanks Balanced salt solution, HBSS, 20 mM HEPES, pH 7.4, 0.1% BSA) for 1 h at room temperature and labeled cells were dispensed into either black 96 well or 384 well assay plates (Greiner). Calcium mobilization induced by urotensin or test compounds was measured in the Flexstation 384 (excitation, 485 nm; emission, 525 nm) for 70 seconds. Agonist activity was determined by direct addition of ligand or peptides, while antagonists were identified by spiking the cells with test compounds prior to urotensin addition. A dose response curve (compound concentration versus % maximum response for urotensin) was determined for each active agonist and antagonist and was fitted to a four parameter logistic equation using SoftmaxPro 4.8 (Molecular Devices), from which EC50% and IC₅₀% values were calculated.

### 2.4. Fluorescence Polarization Assay: NCoA-1 Binding Affinities of Peptides

A fluorescence polarization (FP) assay was established to determine the NCoA-1 binding affinities of the peptides (M. Seitz, L. T. Maillard, D. Obrecht, J. A. Robinson, ChemBioChem 2008, 9, 1318) starting with the K_{D}-determination of the FluoSTAT6 (N-terminal fluorescein-labeled STAT6 794-814 peptide)—NCoA-1 complex: Solutions containing FluoSTAT6 (1 μM final concentration) and NCoA-1 (0-14 μM final concentration) were prepared in HEPES buffer (10 mM HEPES, 150 mM NaCl, 3.4 mM EDTA, 0.1% BSA) and dispensed in a black 96 well plate (Greiner). The mixtures were shaken thoroughly. Fluorescence polarization was measured on a SPECTRAmax M5 spectrometer (Molecular device) following 5-30 mM incubation at room temperature. FluoSTAT6 was excited at 490 nm and emission polarization was detected at 525 nm. 40 intensity measurements were collected for each well, 20 at horizontal position of the dynamic polarizer, 20 at parallel position. As the fraction of FluoSTAT6 bound to NCoA-1 is correlated to the fluorescence polarization (F_{P}), after normalization, the fraction of bound FluoSTAT6 (B) was determined, and the K_{D} was calculated according to B=[(1+P/R+K_{D}/R)−((1+P/R+K_{D}/R)²−4P/R)^{0.5}]/2, wherein P is the total probe concentration, R the total protein concentration and K_{D} the dissociation constant (M. H. Roehrl, J. Y. Wang, G. Wagner, Biochemistry 2004, 3, 16056).

To determine the K, values of potential inhibitors of the STAT6/NCoA-1 interaction (competition fluorescence polarization) the compounds (dilution series from 0 to 100 μM final concentration) dissolved in HEPES buffer (10 mM HEPES, 150 mM NaCl, 3.4 mM EDTA, 0.1% BSA) were dispensed in a black 96 well plate (Greiner) and FluoSTAT6 (200 nM final concentration) in HEPES buffer was added. The mixture was completed by a HEPES buffer solution of NCoA-1 (1 μM final concentration) and processed as described above. STAT6Y (C-terminal tyrosine extended STAT6 794-814 peptide) was used as a positive control. As the total fluorescence intensity of FluoSTAT6 remains similar for all samples, the fraction of peptide bound to NCoA-1 is correlated to the fluorescence polarization.

Following normalization data were fitted with IGORpro software® (WaveMetrics, Lake Oswego, Oreg., USA) to a sigmoid equation to determine IC₅₀ values. The Kᵢ values were calculated from IC₅₀ values according to the method described by Nikolovska-Coleska et al. Anal. Biochem., 2004, 332, 261).

### 2.5. Cytotoxicity Assay

The cytotoxicity of the peptides to HELA cells (Acc57) and COS-7 cells (CRL-1651) was determined using the MTT reduction assay. Briefly, the method was as follows: 7000 HELA cells/well and 4500 COS-7 cells/well were seeded and grown in 96-well microtiter plates for 24 h at 37° C. at 5% CO₂. Thereafter, time zero (Tz) was determined by MTT reduction (see below). The supernatant of the remaining wells was discarded, and fresh medium and compounds in serial dilutions (12.5, 25 and 50 μM, triplicates) were pipetted into the wells. After incubation of the cells for 48 h at 37° C. at 5% CO₂ the supernatant was discarded again and 100 μL MTT reagent (0.5 mg/mL in RPMI1640 and DMEM, respectively)/well was added. Following incubation at 37° C. for 2 h the media were aspirated and the cells were spiked (100 μl isopropanol/well). The absorbance of the solubilized formazan was measured at 595 nm (OD₅₉₅peptide). For each concentration averages were calculated from triplicates. The percentage of growth was calculated as follows: (OD₅₉₅peptide-OD₅₉₅Tz-OD₅₉₅Empty well)/(OD₅₉₅Tz-OD₅₉₅Empty well)×100%. The GI₅₀ (Growth Inhibition) concentrations were calculated for each peptide by using a trend line function for the concentrations (50, 25, 12.5 and 0 μM), the corresponding percentages and the value 50, (=TREND (C₅₀:C₀,%₅₀:%₀,50).

### 2.6. Hemolysis

The peptides were tested for their hemolytic activity against human red blood cells (hRBC). Fresh hRBC were washed three times with phosphate buffered saline (PBS) and centrifuged for 10 min at 2000×g. Compounds (100 μM) were incubated with 20% hRBC (v/v) for 1 h at 37° C. The final erythrocyte concentration was approximately 0.9×10⁹ cells/mL. A value of 0% and 100% cell lyses, respectively, was determined by incubation of hRBC in the presence of PBS alone and 0.1% Triton X-100 in H₂O, respectively. The samples were centrifuged, the supernatants were 20-fold diluted in PBS buffer and the optical densities (OD) were measured at 540 nm. The 100% lyses value (OD₅₄₀H₂O) gave an OD₅₄₀ of approximately 1.3-1.8. Percent hemolysis was calculated as follows:

(OD₅₄₀peptide/OD₅₄₀H₂O)×100%.

### 2.7. Plasma Stability

The stability of the peptides in human and mouse plasma was determined by applying the following method: 315 μl/deep well of freshly thawed human plasma (Basler Blutspende-dienst) and mouse plasma (Harlan Sera-Lab, UK), respectively, were spiked with 35 μl/well of compound in PBS (100 μM, triplicate) and incubated at 37° C. At t=0, 15, 30, 60, 120 and 240 min aliquots of 50 μl were transferred to filtration plate wells containing 150 μl/well of acetonitrile. Following shaking for 2 min the occurred suspensions were filtrated by vacuum and finally, 100 μl of each filtrate were transferred to a propylene microtiter plate, and analyzed by LC/MS as follows: Column: Waters, XBridge C18, mobile phases: (A) water+0.1% formic acid and (B) acetonitrile/water, 95/5 (v/v)+0.1% formic acid, gradient: 5%-100% (B) in 2 minutes, electrospray ionization, MRM detection (triple quadrupole). The peak areas were determined and triplicate values were averaged. The stability was expressed in percent of the initial value at t=0. (tₓ/t₀×100). By using the TREND function of EXCEL (Microsoft Office 2003) T_{1/2} were determined.

TABLE 1

| Ex. | EC 50% [nM], Urotensin II receptor |
|---|---|
| 28 | <2 |
| 29 | <2 |
| 30 | <2 |
| 31 | <2 |
| 33 | <2 |
| 34 | <2 |

TABLE 2

| Ex. | IC 50% [nM] ± SD, Urotensin II receptor |
|---|---|
| 32 | 0.03 ± 0.01 |
| 35 | 0.2 ± 0.01 |

TABLE 3

| Ex. | Kᵢ [μM], peptide binding to NCoA-1 |
|---|---|
| 1 | 0.7 |
| 2 | 4.7 |
| 3 | 6.2 |
| 4 | 15 |
| 5 | 3.8 |
| 6 | 3.6 |
| 7 | 7.1 |
| 8 | 3.0 |
| 9 | 1.5 |
| 10 | 3.1 |
| 11 | 8.7 |
| 12 | 9.3 |
| 13 | 2.0 |
| 14 | 1.4 |
| 15 | 3.1 |
| 16 | 6.6 |
| 17 | 15.8 |
| 18 | 3.3 |
| 19 | 3.7 |
| 20 | 11.5 |
| 21 | 19.6 |
| 22 | 8.9 |
| 23 | 16.4 |
| 24 | 24.3 |
| 25 | 6.0 |
| 26 | 7.7 |
| 27 | 7.9 |

TABLE 4

| | Cytotoxicity | | Hemolysis | Plasmastability | |
|---|---|---|---|---|---|
| | Hela Cells | Cos-7 Cells | at 100 μM | human pl. | mouse pl. |
| Ex. | GI₅₀ [μM] | GI₅₀ [μM] | [%] | T_{1/2} [min] | T_{1/2} [min] |
| 28 | >50 | >50 | 0 | 240 | 240 |
| 29 | >50 | >50 | 0 | 240 | 240 |
| 30 | >50 | >50 | 0 | 240 | 240 |
| 31 | >50 | >50 | 0 | 240 | 240 |
| 32 | >50 | >50 | 0 | 240 | 240 |
| 33 | >50 | >50 | 0 | 240 | 240 |
| 34 | >50 | >50 | 0 | 240 | 240 |
| 35 | >50 | >50 | 0 | 240 | 240 |

1. Compounds of the general formula 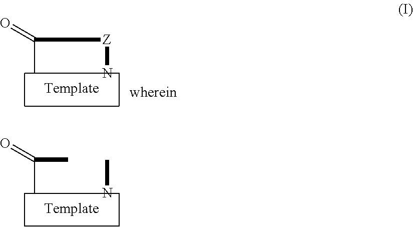
is a group of one of the formulae 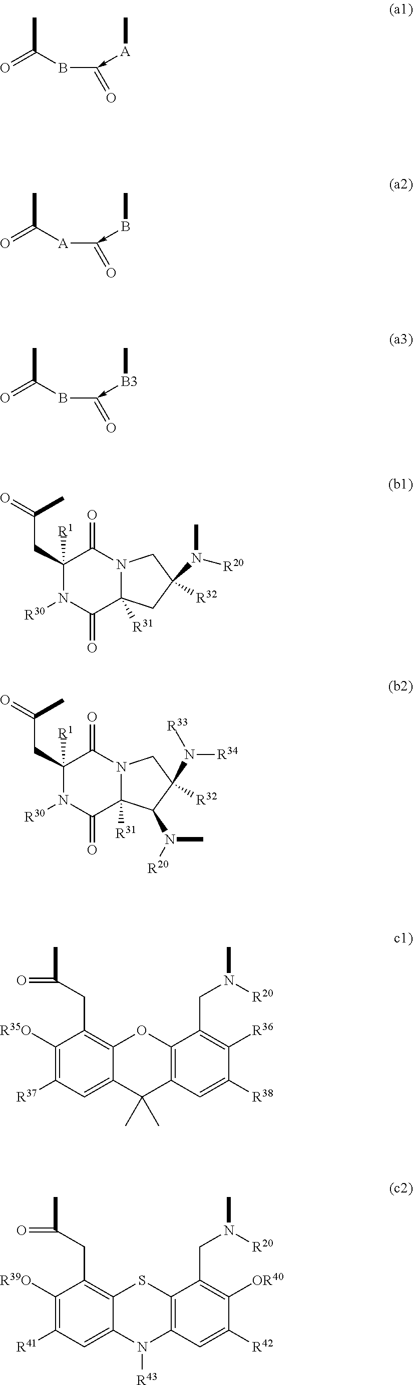 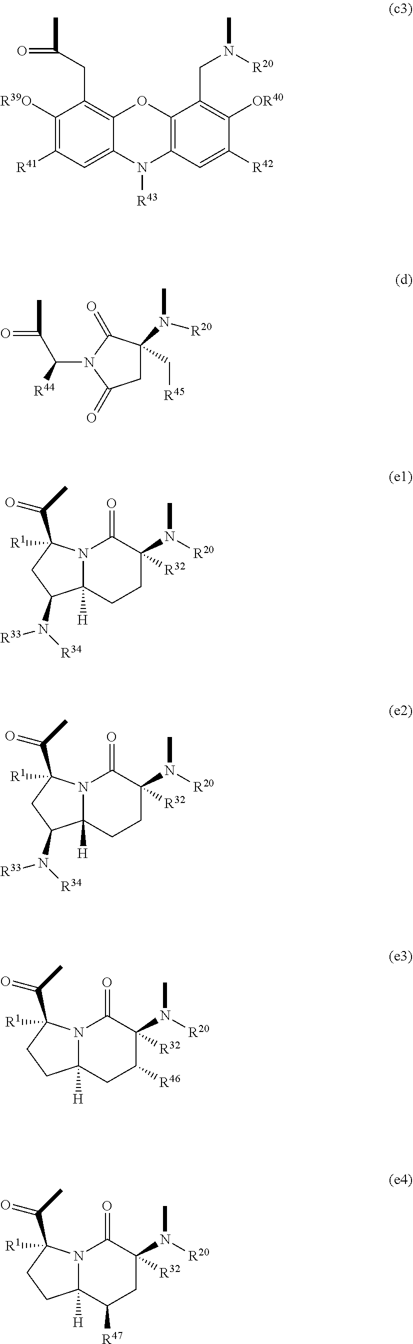 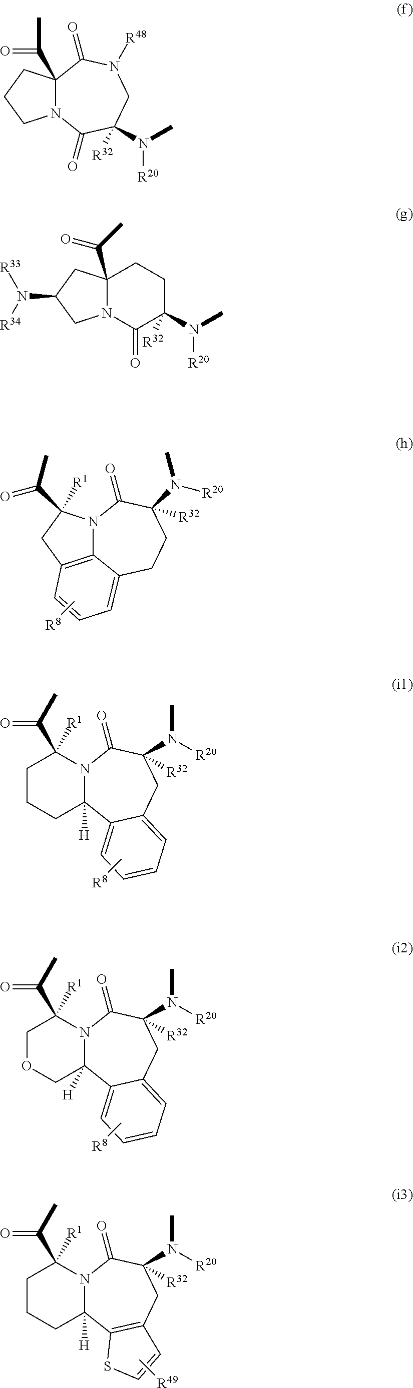 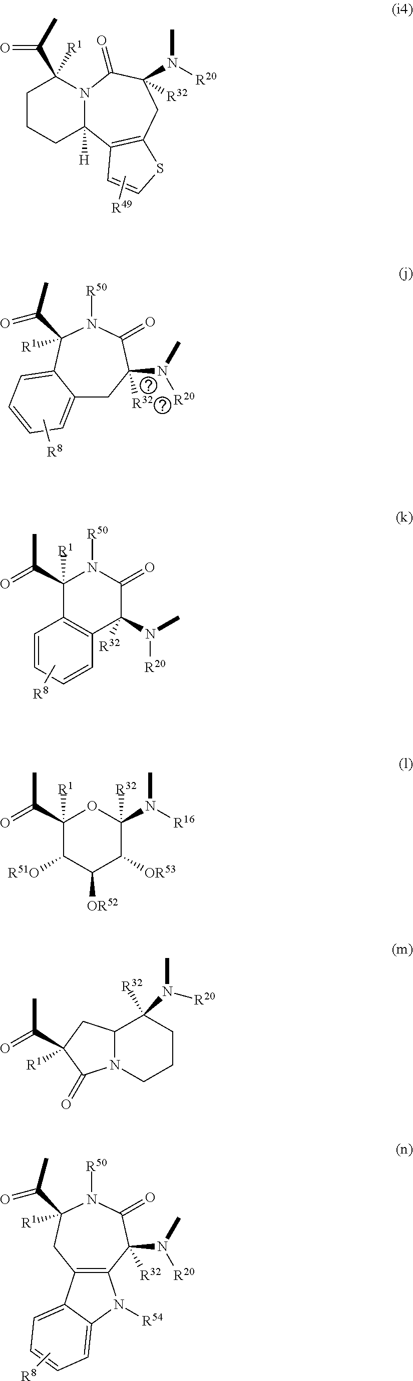 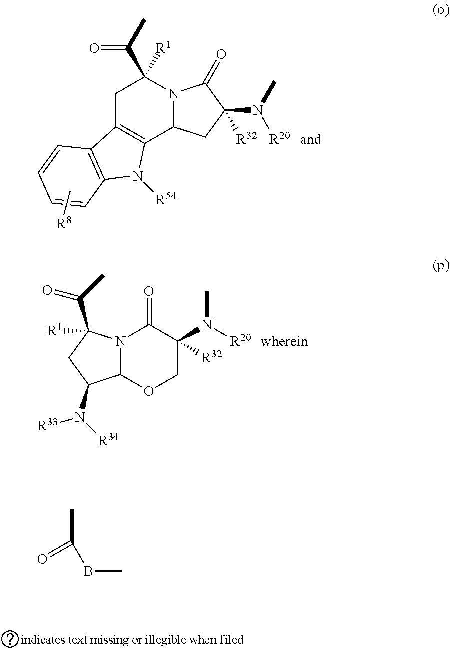
is Gly or the residue of an L-α-amino acid with B being a residue of formula —NR²⁰CH(R⁷¹)— or the enantiomer of one of the groups A1 to A69 and A105 as defined hereinafter; 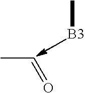
is Gly or the residue of a D-α-amino acid with B3 being a residue of formula —NR²⁰CH(R⁷¹)—; 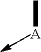
is a group of one of the formulae 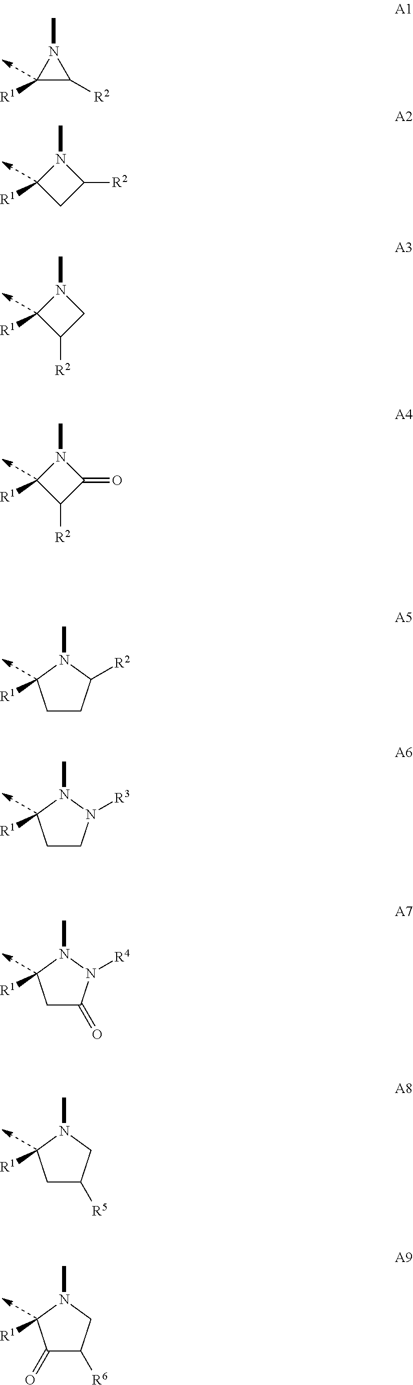 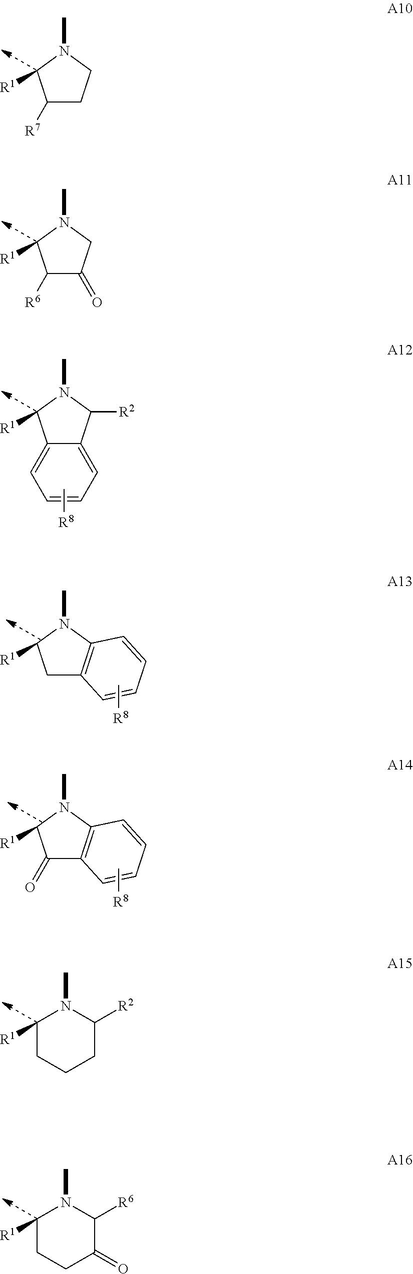 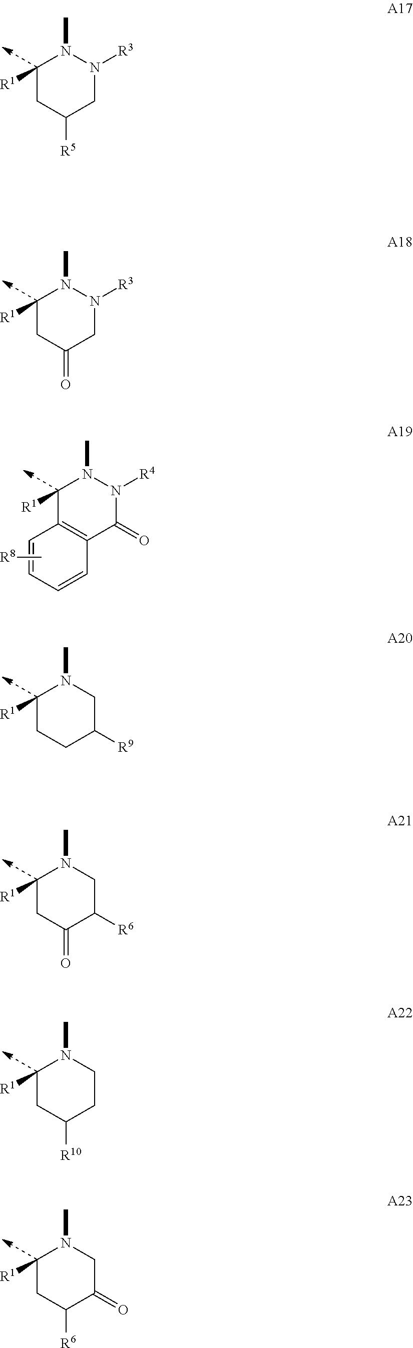 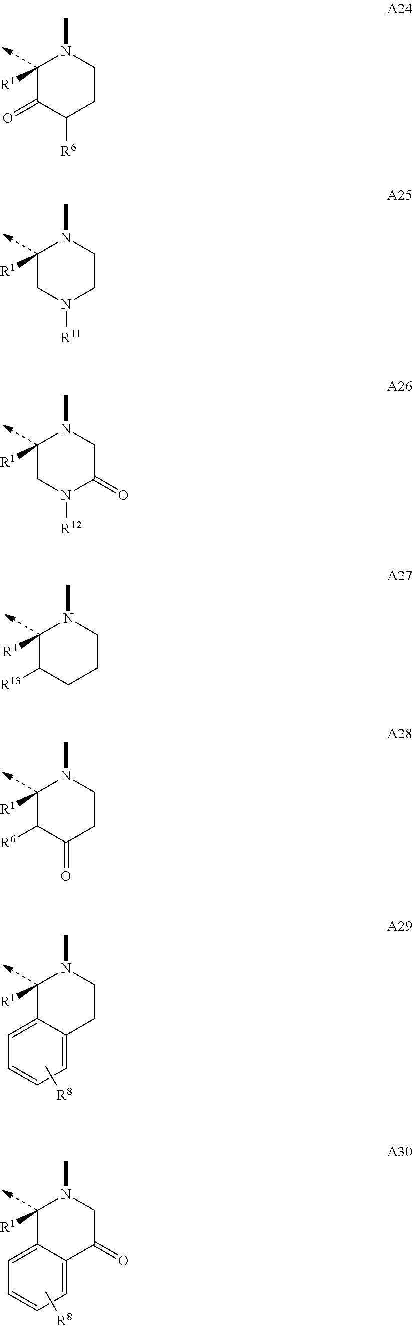 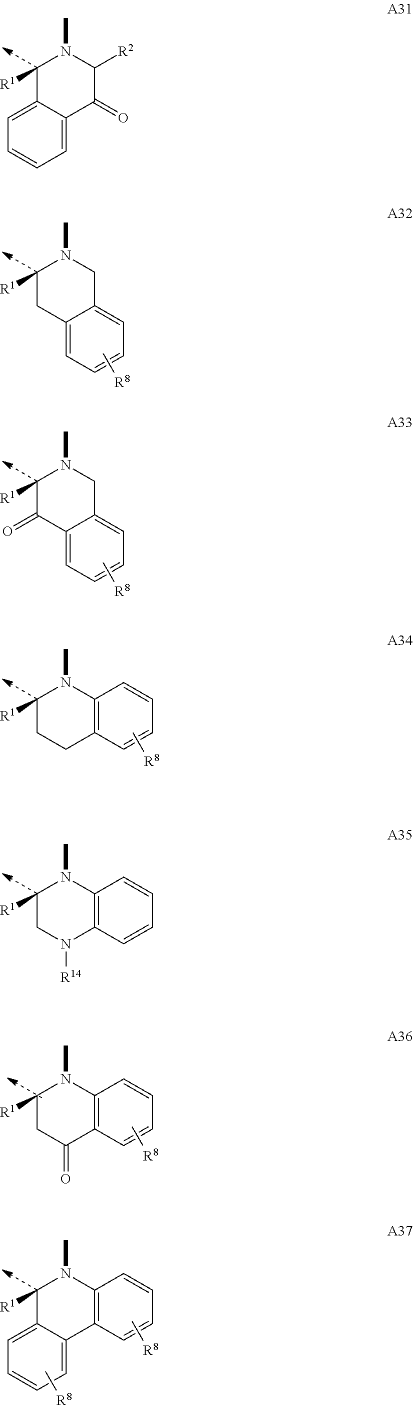 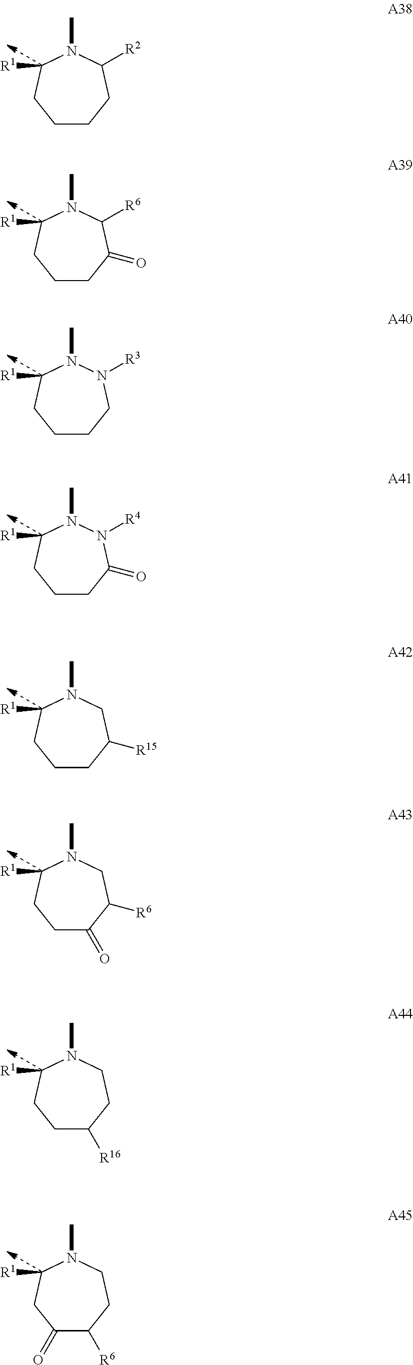 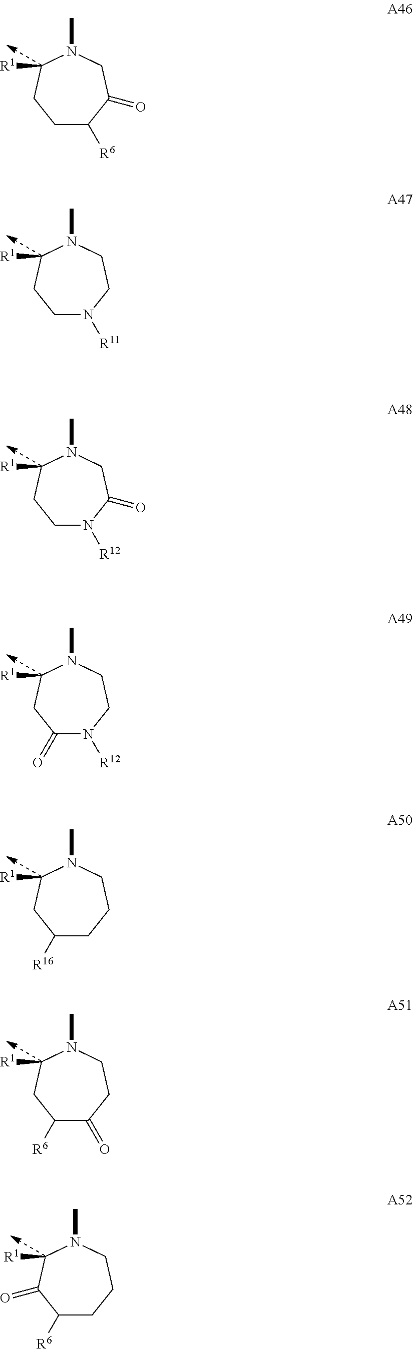 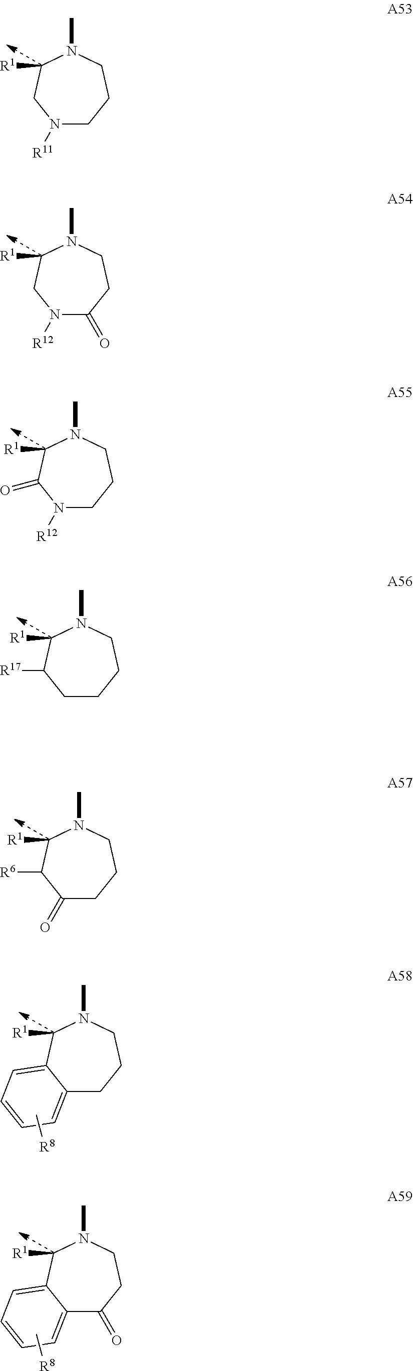 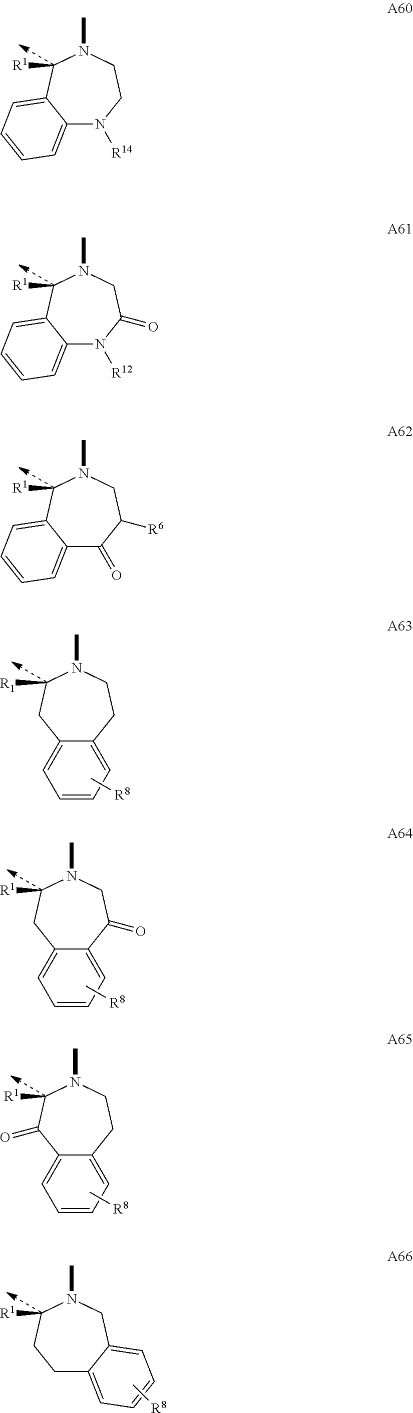 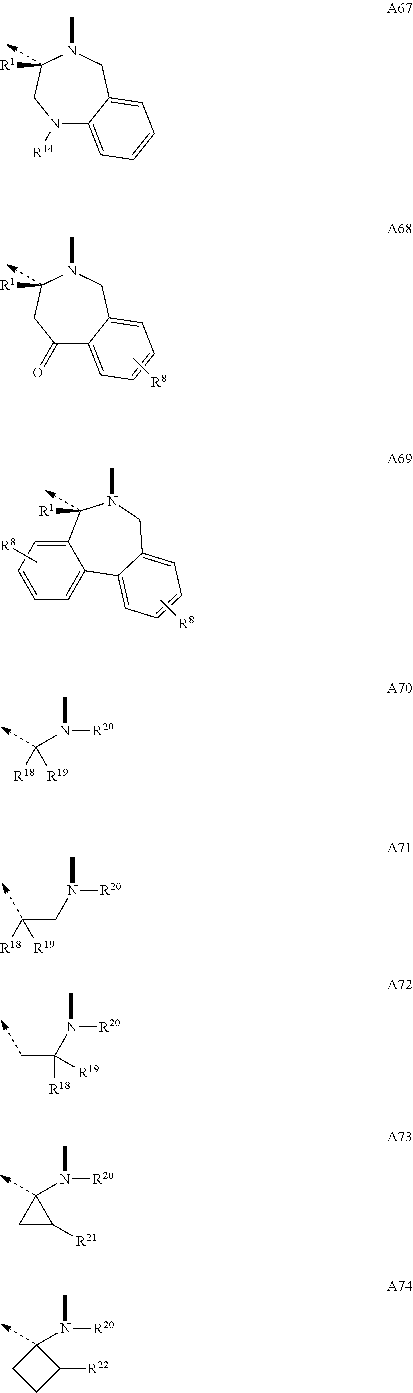 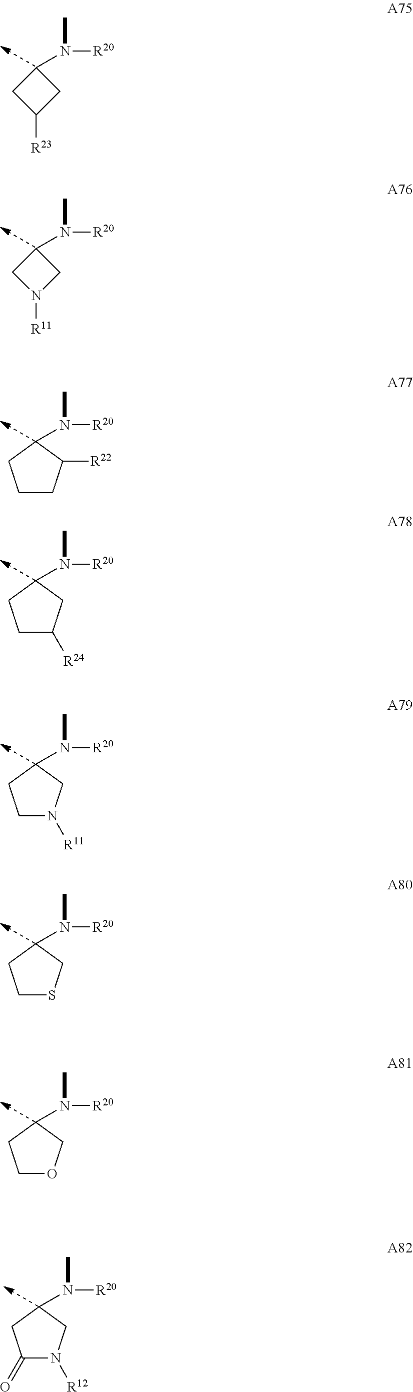 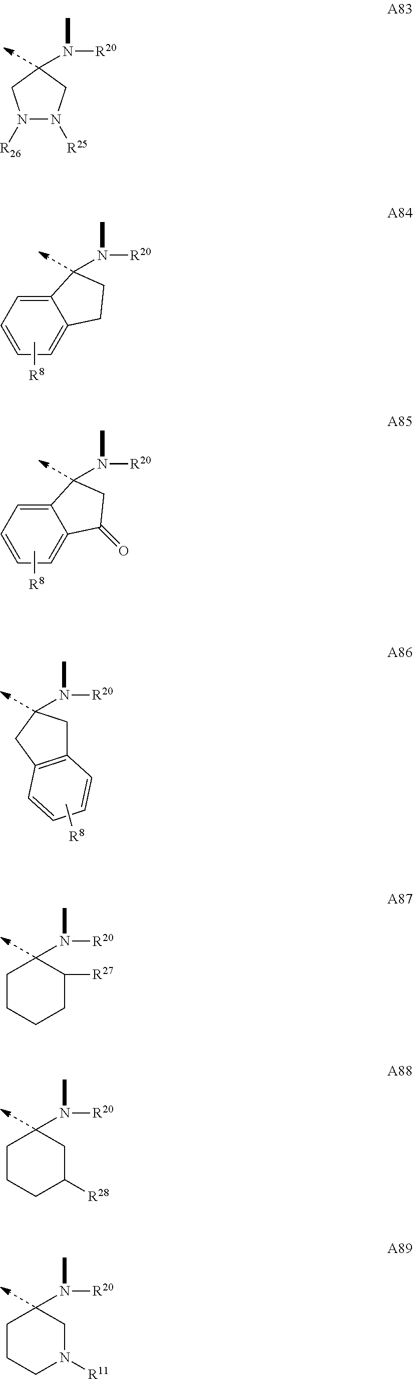 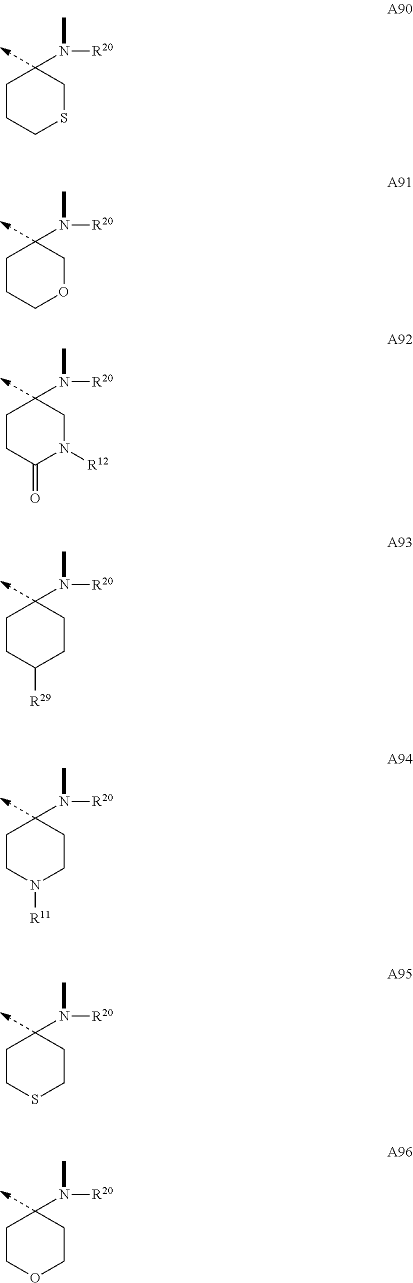 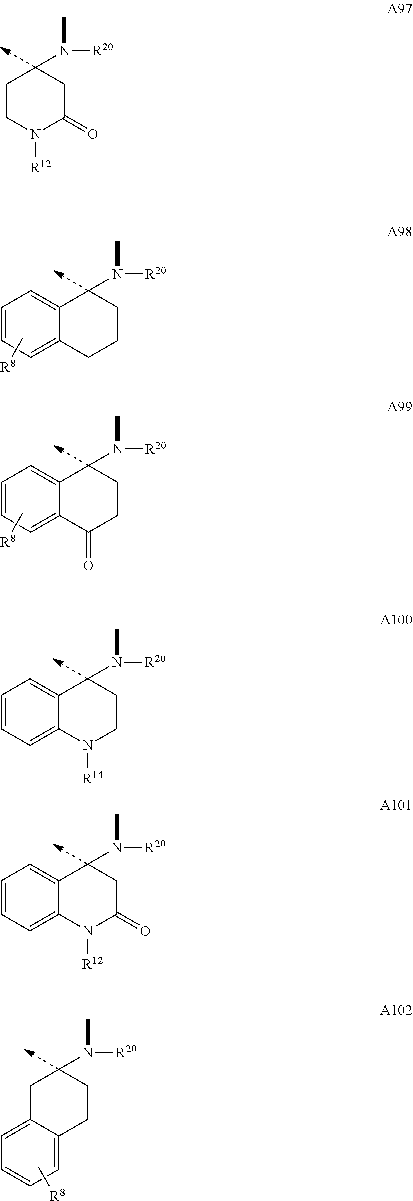 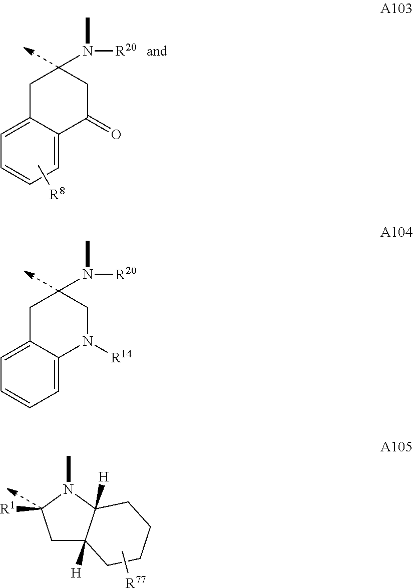
R¹ is H; lower alkyl; or aryl-lower alkyl;
R² is H; alkyl; alkenyl; —(CH₂)ₘ(CHR⁶¹)ₛOR³⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ₒ(CHR⁶¹)ₛCOOR⁵⁷;
—(CH₂)ₒ(CHR⁶¹)ₛCONR⁵⁸R⁵⁹; —(CH₂)ₒ(CHR⁶¹)ₛPO(OR⁶⁶)₂;
—(CH₂)ₒ(CHR⁶¹)ₛSO₂R⁶²; or —(CH₂)ₒ(CHR⁶¹)ₛR⁷⁷;
R³ is H; alkyl; alkenyl; —(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ₒ(CHR⁶¹)ₛCOOR⁵⁷;
—(CH₂)ₒ(CHR⁶¹)ₛCONR⁵⁸R⁵⁹; —(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R⁴ is H; alkyl; alkenyl; —(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴;
—(CH₂)ₘ(CHR⁶¹)ₛOCONR³³R⁷⁵; —(CH₂)ₘ(CHR⁶¹)ₛNR²⁰CONR³³R⁸²;
—(CH₂)ₚ(CHR⁶¹)ₛCOOR⁵⁷; —(CH₂)ₚ(CHR⁶¹)ₛCONR⁵⁸R⁵⁹; —(CH₂)ₚ(CHR⁶¹)ₛPO(OR⁶⁰)₂;
—(CH₂)ₚ(CHR⁶¹)ₛSO₂R⁶²; or —(CH₂)ₒ(CHR⁶¹)ₛC₆H₄R^{S};
R⁵ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R⁶ is H; alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R⁷ is alkyl; alkenyl; —(CH₂)_{q}(CHR⁶¹)ₛOR⁵⁵; —(CH₂)_{q}(CHR⁶¹)ₛNR³³R³⁴;
—(CH₂)_{q}(CHR⁶¹)ₛOCONR³³R⁷⁵; —(CH₂)_{q}(CHR⁶¹)ₛNR²⁰CONR³³R⁸²;
—(CH₂)ᵣ(CHR⁶¹)ₛCOOR⁵⁷; —(CH₂)ᵣ(CHR⁶¹)ₛCONR⁵⁸R⁵⁹;
—(CH₂)ᵣ(CHR⁶¹)ₛPO(OR⁶⁰)₂;
—(CH₂)ᵣ(CHR⁶¹)ₛSO₂R⁶²; or —(CH₂)ᵣ(CHR⁶¹)ₛC₆H₄R^{S};
R^{S} is H; Cl; F; CF₃; NO₂; lower alkyl; lower alkenyl; aryl; aryl-lower alkyl;
—(CH₂)ₒ(CHR⁶¹)ₛOR⁵⁵; —(CH₂)ₒ(CHR⁶¹)ₛSR⁵⁶; —(CH₂)ₒ(CHR⁶¹)NR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; —(CH₂)ₒ(CHR⁶¹)ₛCOR⁶⁴;
R⁹ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R⁸⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)₃PO(OR⁶⁰)₂;
—(CH₂)ₒ(CHR⁶¹)ₛSO₂R⁶²; or —(CH₂)ₒ(CHR⁶¹)ₛC₆H₄R^{S};
R¹⁰ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R¹¹ is H; alkyl; alkenyl; —(CH₂)ₘ(CHR⁶¹)ₛOR⁵⁵; —(CH₂)ₘ(CHR⁶¹)ₛNR³³R³⁴;
—(CH₂)ₒ(CHR⁶¹)ₛOCONR³³R⁷⁵; —(CH₂)ₘ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R¹² is H; alkyl; alkenyl; —(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ᵣ(CHR⁶¹)ₛCOOR⁵⁷;
—(CH₂)ᵣ(CHR⁶¹)ₛCONR⁵⁸R⁵⁹; —(CH₂)ᵣ(CHR⁶¹)ₛPO(OR⁶⁰)₂; —(CH₂)ᵣ(CHR⁶¹)ₛSO₂R⁶²;
or —(CH₂)ᵣ(CHR⁶¹)ₛC₆H₄R^{S};
R¹³ is alkyl; alkenyl; —(CH₂)_{q}(CHR⁶¹)ₛOR⁵⁵; —(CH₂)_{q}(CHR⁶¹)ₛSR⁵⁶;
—(CH₂)_{q}(CHR⁶¹)ₛNR³³R³⁴;
—(CH₂)_{q}(CHR⁶¹)ₛOCONR³³R⁷⁵; —(CH₂)_{q}(CHR⁶¹)ₛNR²⁰CONR³³R⁸²;
—(CH₂)_{q}(CHR⁶¹)ₛCOOR⁵⁷; —(CH₂)_{q}(CHR⁶¹)ₛCONR⁵⁸R⁵⁹;
—(CH₂)_{q}(CHR⁶¹)ₛPO(OR⁶⁰)₂;
—(CH₂)_{q}(CHR⁶¹)ₛSO₂R⁶²; or —(CH₂)_{q}(CHR⁶¹)ₛC₆H₄R^{S};
R¹⁴ is H; alkyl; alkenyl; —(CH₂)ₘ(CHR⁶¹)ₛOR⁵⁵; —(CH₂)ₘ(CHR⁶¹)ₛNR³³R³⁴;
—(CH₂)ₘ(CHR⁶¹)ₛOCONR³³R⁷⁵; —(CH₂)ₘ(CHR⁶¹)ₛNR²⁰CONR³³R⁸²;
—(CH₂)_{q}(CHR⁶¹)ₛCOOR⁵⁷; —(CH₂)_{q}(CHR⁶¹)ₛCONR⁵⁸R⁵⁹;
—(CH₂)_{q}(CHR⁶¹)ₛPO(OR⁶⁰)₂;
—(CH₂)_{q}(CHR⁶¹)ₛSOR⁶²; or —(CH₂)_{q}(CHR⁶¹)ₛC₆H₄R^{S};
R¹⁵ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R¹⁶ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R¹⁷ is alkyl; alkenyl; —(CH₂)_{q}(CHR⁶¹)ₛOR⁵⁵; —(CH₂)_{q}(CHR⁶¹)ₛSR⁵⁶;
—(CH₂)_{q}(CHR⁶¹)ₛNR³³R³⁴;
—(CH₂)_{q}(CHR⁶¹)ₛOCONR³³R⁷⁵; —(CH₂)_{q}(CHR⁶¹)ₛNR²⁰CONR³³R⁸²;
—(CH₂)_{q}(CHR⁶¹)ₛCOOR⁵⁷; —(CH₂)_{q}(CHR⁶¹)ₛCONR⁵⁸R⁵⁹;
—(CH₂)_{q}(CHR⁶¹)ₛPO(OR⁶⁰)₂;
—(CH₂)_{q}(CHR⁶¹)ₛSO₂R⁶²; or —(CH₂)_{q}(CHR⁶¹)ₛC₆H₄R^{S};
R¹⁸ is alkyl; alkenyl; —(CH₂)ₚ(CHR⁶¹)ₛOR⁵⁵; —(CH₂)ₚ(CHR⁶¹)ₛSR⁵⁶;
—(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₚ(CHR⁶¹)ₛCOOR⁵⁷; —(CH₂)ₚ(CHR⁶¹)ₛCONR⁵⁸R⁵⁹;
—(CH₂)ₚ(CHR⁶¹)ₛPO(OR⁶⁰)₂;
—(CH₂)ₚ(CHR⁶¹)ₛSO₂R⁶²; or —(CH₂)ₒ(CHR⁶¹)ₛC₆H₄R^{S};
R¹⁹ is lower alkyl; —(CH₂)ₚ(CHR⁶¹)ₛOR⁵⁵; —(CH₂)ₚ(CHR⁶¹)ₛSR⁵⁶;
—(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₚ(CHR⁶¹)ₛCOOR⁵⁷; —(CH₂)ₚ(CHR⁶¹)ₛCONR⁵⁸R⁵⁹;
—(CH₂)ₚ(CHR⁶¹)ₛPO(OR⁶⁰)₂;
—(CH₂)ₚ(CHR⁶¹)ₛSO₂R⁶²; or —(CH₂)ₒ(CHR⁶¹)ₛC₆H₄R^{S}; or
R¹⁸ and R¹⁹ taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R²⁰ is H; alkyl; alkenyl; or aryl-lower alkyl;
R²¹ is H; alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R²² is H; alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R²³ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₒSO₂R⁶²; or —(CH₂)ₒ(CHR⁶¹)ₛC₆H₄R^{S};
R²⁴ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R²⁵ is H; alkyl; alkenyl; —(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ₒ(CHR⁶¹)ₛCOOR⁵⁷;
—(CH₂)ₒ(CHR⁶¹)ₛCONR⁵⁸R⁵⁹; —(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R²⁶ is H; alkyl; alkenyl; —(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ₒ(CHR⁶¹)ₛCOOR⁵⁷;
—(CH₂)ₒ(CHR⁶¹)ₛCONR⁵⁸R⁵⁹; —(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S}; or
R²⁵ and R²⁶ taken together can form: —(CH₂)₂₋₆—; —(CH₂)ᵣO(CH₂)ᵣ—; —(CH₂)ᵣS(CH₂)ᵣ—; or
—(CH₂)ᵣNR⁵⁷(CH₂)ᵣ—;
R²⁷ is H; alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛOCONR³³R⁷⁵;
—(CH₂)ₒ(CHR⁶¹)ₛNR²⁰CONR³³R⁸²; —(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R²⁸ is alkyl; alkenyl; —(CH₂)ₒ(CHR⁶¹)ₛ—OR⁵⁵; —(CH₂)ₒ(CHR⁶¹)ₛSR⁵⁶; —(CH₂)ₒ(CHR⁶¹)ₛ
NR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R²⁹ is alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R³⁰ is H; alkyl; alkenyl; or aryl-lower alkyl;
R³¹ is H; alkyl; alkenyl; —(CH₂)ₚ(CHR⁶¹)ₛOR⁵⁵; —(CH₂)ₚ(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R³² is H; lower alkyl; or aryl-lower alkyl;
R³³ is H; alkyl, alkenyl; —(CH₂)ₘ(CHR⁶¹)ₛOR⁵⁵; —(CH₂)ₘ(CHR⁶¹)ₛNR³⁴R⁶³;
—(CH₂)ₘ(CHR⁶¹)ₛOCONR⁷⁵R⁸²; —(CH₂)ₘ(CHR⁶¹)ₛNR²⁰CONR⁷⁸R⁸²;
—(CH₂)ₒ(CHR⁶¹)ₛCOR⁶⁴; —(CH₂)ₒ(CHR⁶¹)ₛ—CONR⁵⁸R⁵⁹,
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R³⁴ is H; lower alkyl; aryl, or aryl-lower alkyl; or
R³³ and R³⁴ taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R³⁵ is H; alkyl; alkenyl; —(CH₂)ₘ(CHR⁶¹)ₛOR⁵⁵; —(CH₂)ₘ(CHR⁶¹)ₛNR³³R³⁴;
—(CH₂)ₘ(CHR⁶¹)ₛOCONR³³R⁷⁵; —(CH₂)ₘ(CHR⁶¹)ₛNR²⁰CONR³³R⁸²;
—(CH₂)ₚ(CHR⁶¹)ₛCOOR⁵⁷; —(CH₂)ₚ(CHR⁶¹)ₛCONR⁵⁸R⁵⁹;
—(CH₂)ₚ(CHR⁶¹)ₛPO(OR⁶⁰)₂;
—(CH₂)ₚ(CHR⁶¹)ₛSO₂R⁶²; or —(CH₂)ₚ(CHR⁶¹)ₛC₆H₄R^{S};
R³⁶ is H, alkyl; alkenyl; —(CH₂)ₒ(CHR⁶¹)ₛOR⁵⁵; —(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₚ(CHR⁶¹)ₛCOOR⁵⁷; —(CH₂)ₚ(CHR⁶¹)ₛCONR⁵⁸R⁵⁹;
—(CH₂)ₚ(CHR⁶¹)ₛPO(OR⁶⁰)₂;
—(CH₂)ₚ(CHR⁶¹)ₛSO₂R⁶²; or —(CH₂)ₒ(CHR⁶¹)ₛC₆H₄R^{S};
R³⁷ is H; F; Br; Cl; NO₂; CF₃; lower alkyl; —(CH₂)ₚ(CHR⁶¹)ₛOR⁵⁵;
—(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)₃NR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R³⁸ is H; F; Br; Cl; NO₂; CF₃; alkyl; alkenyl; —(CH₂)ₚ(CHR⁶¹)ₛOR⁵⁵;
—(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R³⁹ is H; alkyl; alkenyl; or aryl-lower alkyl;
R⁴⁹ is H; alkyl; alkenyl; or aryl-lower alkyl;
R⁴¹ is H; F; Br; Cl; NO₂; CF₃; alkyl; alkenyl; —(CH₂)ₚ(CHR⁶¹)ₛOR⁵⁵;
—(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R⁴² is H; F; Br; Cl; NO₂; CF₃; alkyl; alkenyl; —(CH₂)ₚ(CHR⁶¹)ₛOR⁵⁵;
—(CH₂)ₚ(CHR⁶¹)ₛNR³³R³⁴;
—(CH₂)ₚ(CHR⁶¹)ₛOCONR³³R⁷⁵; —(CH₂)ₚ(CHR⁶¹)ₛNR²⁰CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R⁴³ is H; alkyl; alkenyl; —(CH)—(CH₂)ₘ(CHR⁶¹)ₛNR³³R³⁴;
—(CH₂)ₘ(CHR⁶¹)ₛOCONR³³R⁷⁵; —(CH₂)ₘ(CHR⁶¹)ₛNR²⁹CONR³³R⁸²;
—(CH₂)ₒ(CHR⁶¹)ₛCOOR⁵⁷; —(CH₂)ₒ(CHR⁶¹)ₛCONR⁵⁸R⁵⁹;
—(CH₂)ₒ(CHR⁶¹)ₛPO(OR⁶⁰)₂;
—(CH₂)ₒ(CHR⁶¹)ₛSO₂R⁶²; or —(CH₂)ₒ(CHR⁶¹)ₛC₆H₄R^{S};
R⁴⁴ is alkyl; alkenyl; —(CH)—(CH₂)ᵣ(CHR⁶¹)ₛOR⁵⁵; —(CH₂)ᵣ(CHR⁶¹)ₛSR⁵⁶;
—(CH₂)ᵣ(CHR⁶¹)ₛNR³³R³⁴;
—(CH₂)ᵣ(CHR⁶¹)ₛOCONR³³R⁷⁵; —(CH₂)ᵣ(CHR⁶¹)ₛNR²⁰CONR³³R⁸²;
—(CH₂)ᵣ(CHR⁶¹)ₛCOOR⁵⁷; —(CH₂)ᵣ(CHR⁶¹)ₛCONR⁵⁸R⁵⁹;
—(CH₂)ᵣ(CHR⁶¹)ₛPO(OR⁶⁰)₂;
—(CH₂)ᵣ(CHR⁶¹)ₛSO₂R⁶²; or —(CH₂)ᵣ(CHR⁶¹)ₛC₆H₄R^{S};
R⁴⁵ is H; alkyl; alkenyl; —(CH₂)ₒ(CHR⁶¹)ₛOR⁵⁵; —(CH₂)ₒ(CHR⁶¹)ₛSR⁵⁶;
—(CH₂)ₒ(CHR⁶¹)ₛNR³³R³⁴;
—(CH₂)ₒ(CHR⁶¹)ₛOCONR³³R⁷⁵; —(CH₂)ₒ(CHR⁶¹)ₛNR²⁰CONR³³R⁸²;
—(CH₂)ₒ(CHR⁶¹)ₛCOOR⁵⁷; —(CH₂)ₛ(CHR⁶¹)ₛCONR⁵⁸R⁵⁹;
—(CH₂)ₛ(CHR⁶¹)ₛPO(OR⁶⁰)₂;
—(CH₂)ₛ(CHR⁶¹)ₛSO₂R⁶²; or —(CH₂)ₛ(CHR⁶¹)ₛC₆H₄R^{S};
R⁴⁶ is H; alkyl; alkenyl; or —(CH₂)ₒ(CHR⁶¹)ₚC₆H₄R^{S};
R⁴⁷ is H; alkyl; alkenyl; or —(CH₂)ₒ(CHR⁶¹)ₛOR⁵⁵;
R⁴⁸ is H; lower alkyl; lower alkenyl; or aryl-lower alkyl;
R⁴⁹ is H; alkyl; alkenyl; —(CHR⁶¹)ₛCOOR⁵⁷; (CHR⁶¹)ₛCONR⁵⁸R⁵⁹; (CHR⁶¹)ₛPO(OR⁶⁰)₂;
—(CHR⁶¹)ₛSOR⁶²; or —(CHR⁶¹)ₛC₆H₄R^{S};
R⁵⁰ is H; lower alkyl; or aryl-lower alkyl;
R⁵¹ is H; alkyl; alkenyl; —(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ₒ(CHR⁶¹)ₛCOOR⁵⁷;
—(CH₂)ₘ(CHR⁶¹)ₛCONR⁵⁸R⁵⁹; —(CH₂)ₒ(CHR⁶¹)ₚPO(OR⁶⁰)₂;
—(CH₂)ₚ(CHR⁶¹)ₛSO₂R⁶²; or —(CH₂)ₚ(CHR⁶¹)ₛC₆H₄R^{S};
R⁵² is H; alkyl; alkenyl; —(CH)—(CH₂)ₘ(CHR⁶¹)ₛOR⁵⁵; —(CH₂)ₘ(CHR⁶¹)ₛSR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₒ(CHR⁶¹)ₛNR²⁰CONR³³R⁸²; —(CH₂)ₒ(CHR⁶¹)ₛCOOR⁵⁷;
—(CH₂)ₚ(CHR⁶¹)ₛCONR⁵⁸R⁵⁹; —(CH₂)ₒ(CHR⁶¹)ₚPO(OR⁶⁰)₂;
—(CH₂)ₚ(CHR⁶¹)ₒSO₂R⁶²; or —(CH₂)ₚ(CHR⁶¹)ₛC₆H₄R^{S};
R⁵³ is H; alkyl; alkenyl; —(CH₂)ₘ(CHR⁶¹)ₛOR⁵⁵; —(CH₂)ₘ(CHR⁶¹)₅SR⁵⁶;
—(CH₂)ₘ(CHR⁶¹)ₛNR³³R³⁴; —(CH₂)ₘ(CHR⁶¹)ₛOCONR³³R⁷⁵;
—(CH₂)ₘ(CHR⁶¹)ₛNR²⁰CONR³³R⁸²; —(CH₂)ₒ(CHR⁶¹)ₛCOOR⁵⁷;
—(CH₂)ₒ(CHR⁶¹)ₛCONR⁵⁸R⁵⁹; —(CH₂)ₒ(CHR⁶¹)ₚPO(OR⁶⁰)₂;
—(CH₂)ₚ(CHR⁶¹)ₛSO₂R⁶²; or —(CH₂)ₚ(CHR⁶¹)ₛC₆H₄R^{S};
R⁵⁴ is H; alkyl; alkenyl; —(CH₂)ₘ(CHR⁶¹)ₛOR⁵⁵; —(CH₂)ₘ(CHR⁶¹)ₛNR³³R³⁴;
—(CH₂)ₘ(CHR⁶¹)ₛOCONR³³R⁷⁵; —(CH₂)ₘ(CHR⁶¹)ₛNR²⁰CONR³³R⁸²;
—(CH₂)ₒ(CHR⁶¹)COOR⁵⁷; —(CH₂)ₒ(CHR⁶¹)ₛCONR⁵⁸R⁵⁹; or —
(CH₂)ₒ(CHR⁶¹)ₛC₆H₄R^{S};
R⁵⁵ is H; lower alkyl; lower alkenyl; aryl-lower alkyl; —(CH₂)ₘ(CHR⁶¹)ₛOR⁵⁷;
—(CH₂)ₘ(CHR⁶¹)ₛNR³⁴R⁶³; —(CH₂)ₘ(CHR⁶¹)ₛOCONR⁷⁵R⁸²;
—(CH₂)ₘ(CHR⁶¹)ₛNR²⁰CONR⁷⁸R⁸²; —(CH₂)ₒ(CHR⁶¹)ₛ—COR⁶⁴;
—(CH₂)ₒ(CHR⁶¹)COOR⁵⁷; or
—(CH₂)ₒ(CHR⁶¹)ₛCONR⁵⁸R⁵⁹;
R⁵⁶ is H; lower alkyl; lower alkenyl; aryl-lower alkyl; —(CH₂)ₘ(CHR⁶¹)ₛOR⁵⁷;
—(CH₂)ₘ(CHR⁶¹)ₛNR³⁴R⁶³(CH₂)ₘ(CHR⁶¹)ₛOCONR⁷⁵R⁸²;
—(CH₂)ₘ(CHR⁶¹)ₛNR²⁰CONR⁷⁸R⁸²; —(CH₂)ₒ(CH₂)ₒ(CHR⁶¹)ₛ—COR⁶⁴; or
—(CH₂)ₒ(CHR⁶¹)ₛCONR⁵⁸R⁵⁹;
R⁵⁷ is H; lower alkyl; lower alkenyl; aryl lower alkyl; or heteroaryl lower alkyl;
R⁵⁸ is H; lower alkyl; lower alkenyl; aryl; heteroaryl; aryl-lower alkyl; or heteroaryl-lower alkyl;
R⁵⁹ is H; lower alkyl; lower alkenyl; aryl; heteroaryl; aryl-lower alkyl; or heteroaryl-lower alkyl; or
R⁵⁸ and R⁵⁹ taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R⁶⁰ is H; ti lower alkyl; lower alkenyl; aryl; or aryl-lower alkyl;
R⁶¹ is alkyl; alkenyl; aryl; heteroaryl; aryl-lower alkyl; heteroaryl-lower alkyl;
—(CH₂)ₘOR⁵⁵;
—(CH₂)ₘNR³³R³⁴; —(CH₂)ₘOCONR⁷⁵R⁸²; —(CH₂)ₘNR²⁰CONR⁷⁸R⁸²;
—(CH₂)ₒCOOR³⁷;
—(CH₂)ₒNR⁵⁸R⁵⁹; or —(CH₂)ₒPO(COR⁶⁰)₂;
R⁶² is lower alkyl; lower alkenyl; aryl, heteroaryl; or aryl-lower alkyl;
R⁶³ is H; lower alkyl; lower alkenyl; aryl, heteroaryl; aryl-lower alkyl; heteroaryl-lower alkyl;
—COR⁶⁴; —COOR⁵⁷; —CONR⁵⁸R⁵⁹; —SO₂R⁶²; or —PO(OR⁶⁰)₂; or
R³⁴ and R⁶³ taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R⁶⁴ is H; lower alkyl; lower alkenyl; aryl; heteroaryl; aryl-lower alkyl; heteroaryl-lower alkyl;
—(CH₂)ₚ(CHR⁶¹)ₛOR⁶⁵; —(CH₂)ₚ(CHR⁶¹)ₛSR⁶⁶; or —(CH₂)ₚ(CHR⁶¹)ₛNR³⁴R⁶³;
—(CH₂)ₚ(CHR⁶¹)ₛOCONR⁷⁵R⁸²; —(CH₂)ₚ(CHR⁶¹)ₛNR²⁰CONR⁷⁸R⁸²;
R⁶⁵ is H; lower alkyl; lower alkenyl; aryl, aryl-lower alkyl; heteroaryl-lower alkyl;
—COR⁵⁷;
—COOR⁵⁷; or —CONR⁵⁸R⁵⁹;
R⁶⁶ is H; lower alkyl; lower alkenyl; aryl; aryl-lower alkyl; heteroaryl-lower alkyl; or
—CONR⁵⁸R⁵⁹;
R⁶⁷ is H; Cl; Br; F; NO₂; —NR³⁴COR⁵⁷; lower alkyl; or lower alkenyl;
R⁶⁸ is H; Cl; Br; F; NO₂; —NR³⁴COR⁵⁷; lower alkyl; or lower alkenyl;
R⁶⁹ is H; Cl; Br; F; NO₂; —NR³⁴COR⁵⁷; lower alkyl; or lower alkenyl;
R⁷⁰ is H; Cl; Br; F; NO₂; —NR³⁴COR⁵⁷; lower alkyl; or lower alkenyl;
with the proviso that at least two of R⁶⁷, R⁶⁸, R⁶⁹ and R⁷⁰ are H;
R⁷¹ is lower alkyl; lower alkenyl; —(CH₂)ₚ(CHR⁶¹)ₛOR⁷⁵; —(CH₂)ₚ(CHR⁶¹)ₛSR⁷⁵;
—(CH₂)ₚ(CHR⁶¹)ₛNR³³R³⁴; —(CH₂)ₚ(CHR⁶¹)ₛOCONR³³R⁷⁵;
—(CH₂)ₚ(CHR⁶¹)ₛNR²⁰CONR³³R⁸²;
—(CH₂)ₒ(CHR⁶¹)ₛCOOR⁷⁵; —(CH₂)ₚCONR⁵⁸R⁵⁹; —(CH₂)ₚPO(OR⁶²)₂; —(CH₂)ₚSO₂R⁶²; or
—(CH₂)ₒ—C₆R⁶⁷R⁶⁸R⁶⁹R⁷⁰R⁷⁶;
Z is a chain of 8 α-amino acid residues, the positions of said amino acid residues in said chain being counted starting from the N-terminal amino acid, whereby these amino acid residues are, depending on their position in the chains, Gly, Pro or of one of the types
C: —NR²⁰CH(R⁷²)CO—;
D: —NR²⁰CH(R⁷³)CO—;
E: —NR²⁰CH(R⁷⁴)CO—;
F: —NR²⁰CH(R⁸⁴)CO—; and
H: —NR²⁰—CH(CO—)—(CH₂)₄₋₇—CH(CO—)—NR²⁰—;
—NR²⁰—CH(CO—)—(CH₂)ₚSS(CH₂)ₚ—CH(CO—)—NR²⁰—;
—NR²⁰—CH(CO—)—(—(CH₂)ₚNR²⁰CO(CH₂)ₚ—CH(CO—)—NR²⁰—; or
—NR²⁰—CH(CO—)—(—(CH₂)ₚNR²⁰CONR²⁰(CH₂)ₚ—CH(CO—)—NR²⁰—;
R⁷² is H, lower alkyl; lower alkenyl; —(CH₂)ₚ(CHR⁶¹)ₛOR⁸⁵; or —(CH₂)ₚ(CHR⁶¹)ₛSR⁸⁵;
R⁷³ is —(CH₂)ₒR⁷⁷; —(CH₂)ᵣO(CH₂)ₒR⁷⁷; —(CH₂)ᵣS(CH₂)ₒR⁷⁷; or —(CH₂)ᵣNR²⁰(CH₂)ₒR⁷⁷;
R⁷⁴ is —(CH₂)ₚNR⁷⁸R⁷⁹; —(CH₂)ₚNR⁷⁷R⁸⁰; —(CH₂)ₚC(═NR⁸⁰)NR⁷⁸R⁷⁹;
—(CH₂)ₚC(═NOR⁵⁰)NR⁷⁸R⁷⁹;
—(CH₂)ₚC(═NNR⁷⁸R⁷⁹)NR⁷⁸R⁷⁹; —(CH₂)ₚNR⁸⁰C(═NR⁸⁰)NR⁷⁸R⁷⁹;
—(CH₂)ₚN═C(NR⁷⁸R⁸⁰)NR⁷⁹R⁸⁰; —(CH₂)ₚC₆H₄NR⁷⁸R⁷⁹; —(CH₂)ₚC₆H₄NR⁷⁷R⁸⁰;
—(CH₂)ₚC₆H₄C(═NR⁸⁰)NR⁷⁸R⁷⁹; —(CH₂)ₚC₆H₄C(═NOR⁵⁰)NR⁷⁸R⁷⁹;
—(CH₂)ₚC₆H₄C(═NNR⁷⁸R⁷⁹)NR⁷⁸R⁷⁹; —(CH₂)ₚC₆H₄NR⁸⁰C(═NR⁸⁰)NR⁷⁸R⁷⁹;
—(CH₂)ₚC₆H₄N═C(NR⁷⁸R⁸⁰)NR⁷⁹R⁸⁰; —(CH₂)ᵣO(CH₂)ₘNR⁷⁸R⁷⁹;
—(CH₂)ᵣO(CH₂)ₘNR⁷⁷R⁸⁰;
—(CH₂)ᵣO(CH₂)ₚc(═NR⁸⁰)NR⁷⁸R⁷⁹; —(CH₂)ᵣO(CH₂)ₚC(═NOR⁵⁰)NR⁷⁸R⁷⁹;
—(CH₂)ᵣO(CH₂)ₚc(═NNR⁷⁸R⁷⁹)NR⁷⁸R⁷⁹; —(CH₂)ᵣO(CH₂)ₘNR⁸⁰C(═NR⁸⁰)NR⁷⁸R⁷⁹;
—(CH₂)ᵣO(CH₂)ₘN═C(NR⁷⁸R⁸⁰)NR⁷⁹R⁸⁰; —(CH₂)ᵣO(CH₂)ₚC₆H₄CNR⁷⁸R⁷⁹;
—(CH₂)ᵣO(CH₂)ₚC₆H₄C(═NR⁸⁰)NR⁷⁸R⁷⁹; —(CH₂)ᵣO(CH₂)ₚC₆H₄C(═NOR⁵⁰)NR⁷⁸R⁷⁹;
—(CH₂)ᵣo(CH₂)ₚC₆H₄c(═NNR⁷⁸R⁷⁹)NR⁷⁸R⁷⁹;
—(CH₂)ᵣ0(CH₂)ₚC₆H₄NR⁸⁰C(═NR⁸⁰)NR⁷⁸R⁷⁹; —(CH₂)ᵣS(CH₂)ₘNR⁷⁸R⁷⁹;
—(CH₂)ᵣS(CH₂)ₘNR⁷⁷R⁸⁰; —(CH₂)ᵣS(CH₂)ₚC(═NR⁸⁰)NR⁷⁸R⁷⁹;
—(CH₂)ᵣS(CH₂)ₚC(═NOR⁵⁰)NR⁷⁸R⁷⁹; —(CH₂)ᵣS(CH₂)ₚC(═NNR⁷⁸R⁷⁹)NR⁷⁸R⁷⁹;
—(CH₂)ᵣS(CH₂)ₘNR⁸⁰C(═NR⁸⁰)NR⁷⁸R⁷⁹; —(CH₂)ᵣS(CH₂)ₘN═C(NR⁷⁸R⁸⁰)NR⁷⁹R⁸⁰;
—(CH₂)ᵣS(CH₂)ₚC₆H₄CNR⁷⁸R⁷⁹; —(CH₂)ᵣS(CH₂)ₚC₆H₄C(═NR⁸⁰)NR⁷⁸R⁷⁹;
—(CH₂)ᵣS(CH₂)ₚC₆H₄C(═NOR⁵⁰)NR⁷⁸R⁷⁹;
—(CH₂)ᵣS(CH₂)ₚC₆H₄C(═NNR⁷⁸R⁷⁹)NR⁷⁸R⁷⁹;
—(CH₂)ᵣS(CH₂)ₚC₆H₄NR⁸⁰C(═NR⁸⁰)NR⁷⁸R⁷⁹; —(CH₂)ₚNR⁸⁰COR⁶⁴; —(CH₂)ₚNR⁸⁰COR⁷⁷;
—(CH₂)ₚNR⁸⁰CONR⁷⁸R⁷⁹; —(CH₂)ₚC₅H₄NR⁸⁰CONR⁷⁸R⁷⁹
R⁷⁵ is lower alkyl; lower alkenyl; or aryl-lower alkyl; or
R³³ and R⁷⁵ taken together can form: —(CH₂)₂₋₆—; —(CH₂)₂O(CH₂)₂—; —(CH₂)₂S(CH₂)₂—; or
—(CH₂)₂NR⁵⁷(CH₂)₂—; or
R⁷⁵ and R⁸² taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R⁷⁶ is H; lower alkyl; lower alkenyl; aryl-lower alkyl; —(CH₂)ₒR⁷²; —(CH₂)ₒSR⁷²;
—(CH₂)ₒNR³³R³⁴; —(CH₂)ₒOCONR³³R⁷⁵; —(CH₂)ₒNR²⁰CONR³³R⁸²;
—(CH₂)ₒCOOR⁷⁵; —(CH₂)ₒCONR⁵⁸R⁵⁹; —(CH₂)ₒPO(OR⁶⁶)₂; —(CH₂)ₚSO₂R⁶²; or
—(CH₂)ₒCOR⁶⁴;
R⁷⁷ is —C₆R⁶⁷R⁶⁸R⁶⁹R⁷⁰R⁷⁶; or a heteroaryl group of one of the formulae 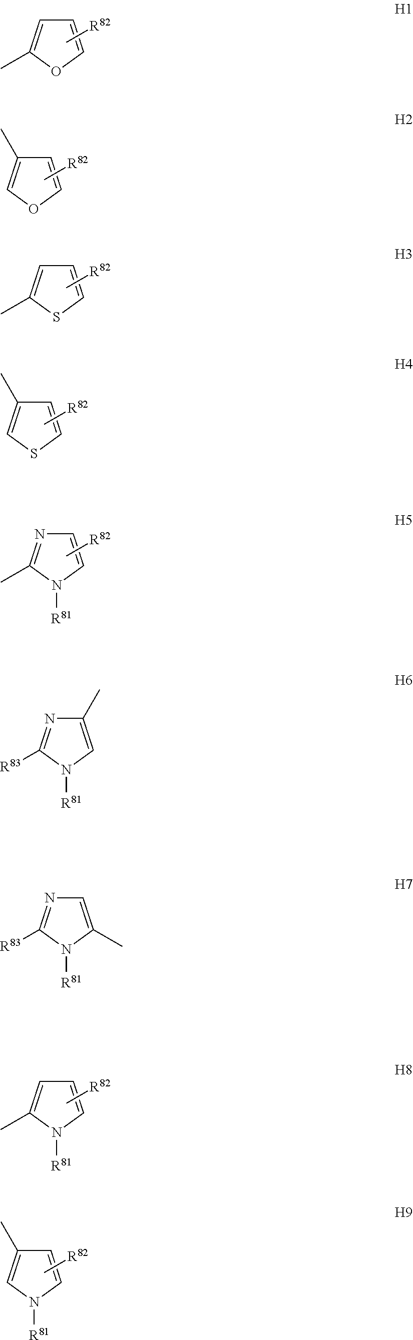 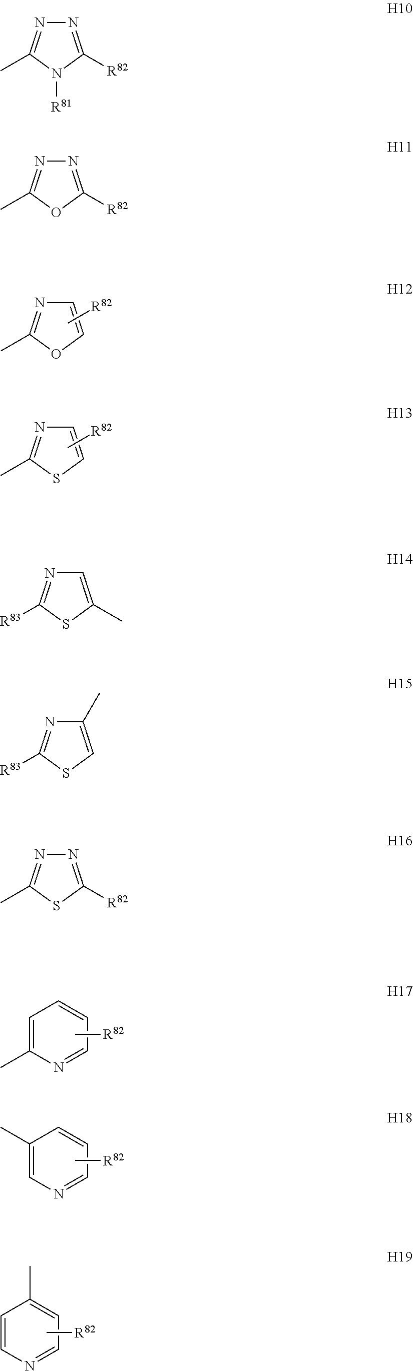 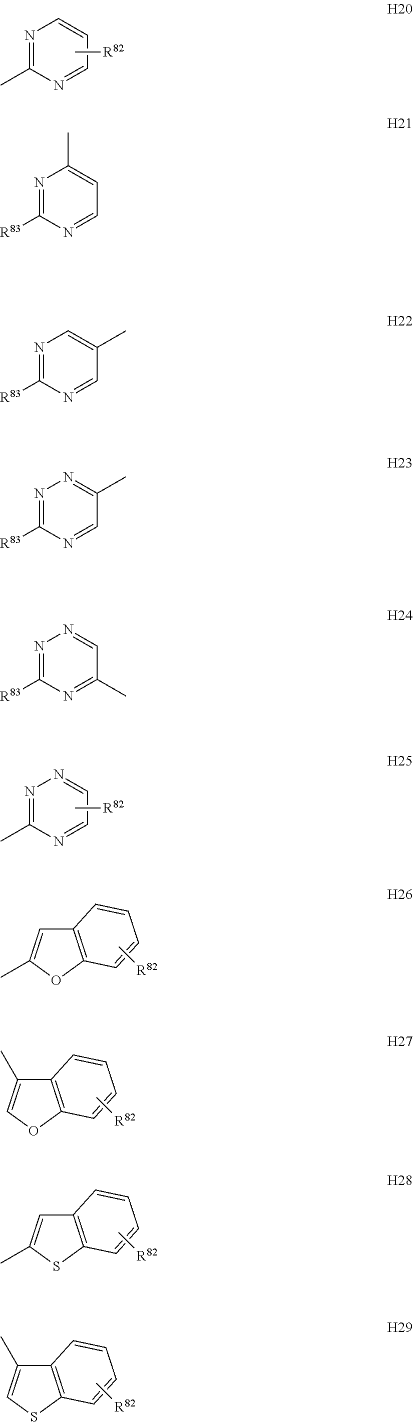 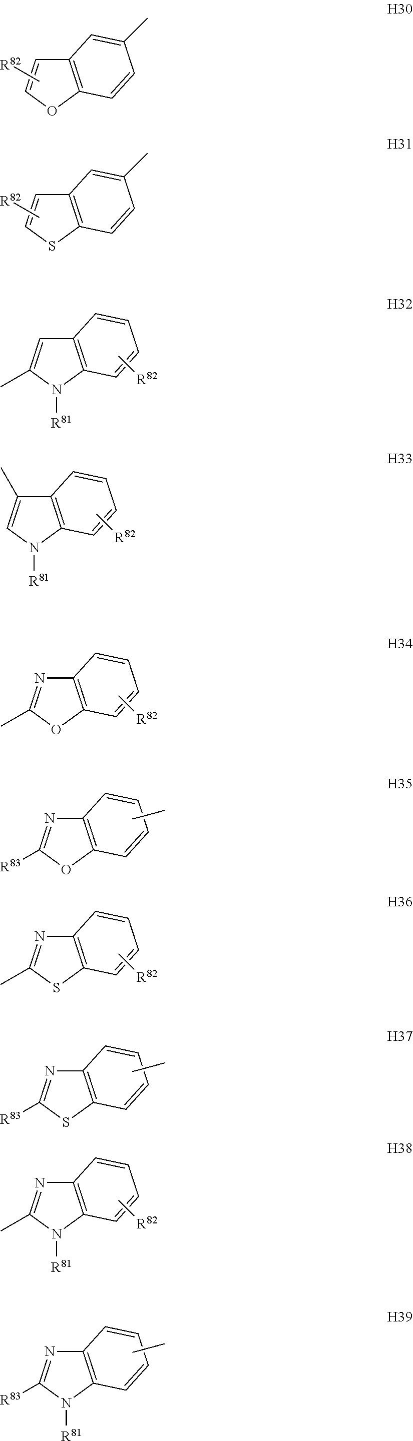 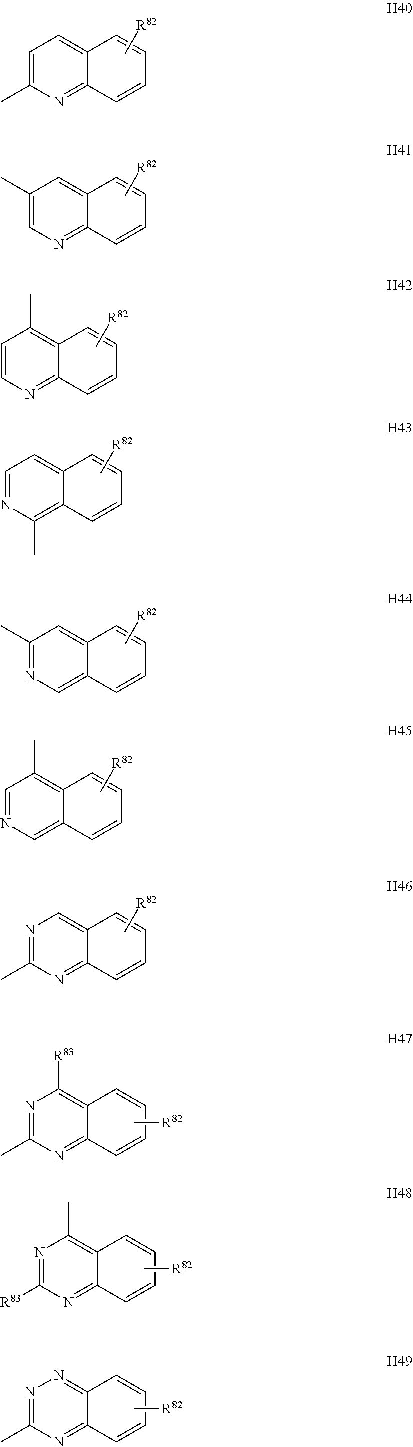 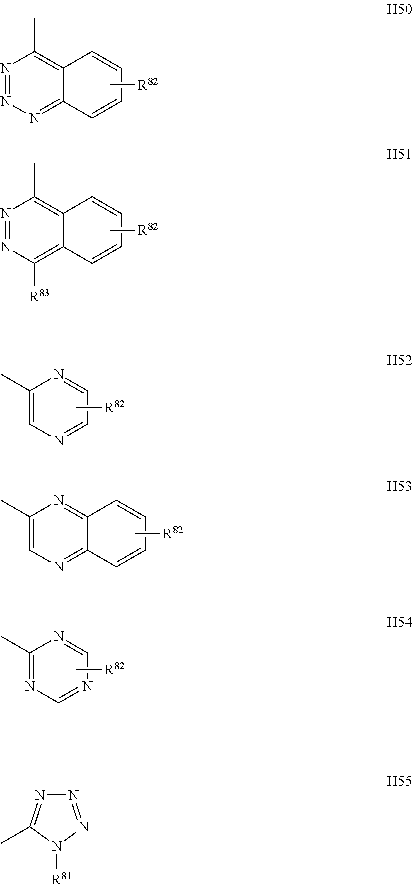
R⁷⁸ is H; lower alkyl; aryl; or aryl-lower alkyl; or
R⁷⁸ and R⁸² taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R⁷⁹ is H; lower alkyl; aryl; or aryl-lower alkyl; or
R⁷⁸ and R⁷⁹, taken together, can be —(CH₂)₂₋₇—; —(CH₂)₂O(CH₂)₂—; or —(CH₂)₂NR⁵⁷(CH₂)₂—;
R⁸⁰ is H; or lower alkyl;
R⁸¹ is H; lower alkyl; or aryl-lower alkyl;
R⁸² is H; lower alkyl; aryl; heteroaryl; or aryl-lower alkyl; or
R³³ and R⁸² taken together can form: —(CH₂)₂₋₆—; —(CH₂)₂O(CH₂)₂—; —(CH₂)₂S(CH₂)₂—; or
—(CH₂)₂NR⁵⁷(CH₂)₂—;
R⁸³ is H; lower alkyl; aryl; or —NR⁷⁸R⁷⁹;
R⁸⁴ is —(CH₂)ₘ(CHR₆₁)ₛOH; —(CH₂)pCOOR₈₀; —(CH₂)ₘ(CHR₆₁)ₛSH; —(CH₂)ₚCONR⁷⁸R⁷⁹; —
(CH₂)ₚNR⁸⁰CONR⁷⁸R⁷⁹; —(CH₂)ₚC₆H₄CONR⁷⁸R⁷⁹; or —(CH₂)ₚC₆H₄NR⁸⁰CONR⁷⁸R⁷⁹;
R⁸⁵ is lower alkyl; or lower alkenyl;
m is 2-4; o is 0-4; p is 1-4; q is 0-2; r is 1 or 2; and s is 0 or 1;
with the proviso that in said chain Z of n α-amino acid residues the amino acid residues in positions 1 to 8 are:
P1: of type C, or of type D, or of type E, or of type F;
P2: of type C, or of type F;
P3: of type C, or of type D;
P4: of type C, or of type D, or of type F, or the residue is Gly;
P5: of type C, or of type D, or of type E, or of type F, or the residue is Gly or Pro;
P6: of type C, or of type D; or the residue is Pro;
P7: of type C, or of type D, or of type F;
P8: of type C, or of type D, or of type E, or of type F; or
P2 and P7, taken together, can form a group of type H;
at P4 and P5 also D-isomers being possible;
and pharmaceutically acceptable salts thereof.

2. Compounds according to claim 1 wherein 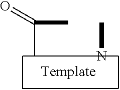
is a group of formula (a1) or (a2) or (a3).

3. Compounds according to claim 2 wherein A is a group of one of the formulae A1 to A69 and A105;
R¹ is hydrogen or lower alkyl;
R² is H; lower alkyl; lower alkenyl; —(CH₂)ₘOR⁵⁵ (where R⁵⁵ is lower alkyl; or lower alkenyl); —(CH₂)ₘSR⁵⁶ (where R⁵⁶ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; lower allyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴(where: R²⁰ is H; or lower alkyl; R⁶⁴ is lower alkyl; or lower alkenyl); (CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R³ is H; lower alkyl; lower alkenyl; —(CH₂)ₘOR⁵⁵ (where R⁵⁵ is lower alkyl; or lower alkenyl); —(CH₂)ₘSR⁵⁶ (where R⁵⁶ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy). R⁴ is H; lower alkyl; lower alkenyl; —(CH₂)ₘOR⁵⁵ (where R⁵⁵ is lower allyl; or lower alkenyl); —(CH₂)ₘSR⁵⁶ (where R⁵⁶ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴(where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy). R⁵ is lower allyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); (CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower lower alkyl; R³³ is H; or lower alkyl; or
lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where: R²⁰ is H; or lower alkyl; R⁶⁴ is alkyl; alkenyl; aryl; aryl-lower alkyl; or heteroaryl-lower alkyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy):
R⁶ is H; lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy); R⁷ is lower alkyl; lower alkenyl; —(CH₂)_{q}OR⁵⁵ (where R⁵⁵ is lower alkyl; or lower alkenyl); —(CH₂)_{q}SR⁵⁶ (where R⁵⁶ is lower alkyl; or lower alkenyl); —(CH₂)_{q}NR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}OCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; (CH₂)₂NR⁵⁷(CH₂)₂—; where R⁵⁷ is H; or lower alkyl); —(CH₂)_{q}NR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}N(R²⁰)COR⁶⁴(where: R²⁰ is H; or lower allyl; R⁶⁴ is lower alkyl; or lower alkenyl); —(CH₂)ᵣCOOR⁵⁷ (where R⁵⁷ is lower alkyl; or lower alkenyl); —(CH₂)_{q}CONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ᵣPO(OR⁶⁰)₂ (where R⁶⁰ is lower alkyl; or lower alkenyl); —(CH₂)ᵣSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R^{S} is H; F; Cl; CF₃; lower alkyl; lower alkenyl; —(CH₂)ₒR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower allyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R⁹ is lower alkyl; lower alkenyl; —(CH₂)ₒR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR ³³CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower allyl); —(CH₂)ₒN(R²⁰)COR⁶⁴(where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower allyl; or lower alkenyl); or —(CH₂)_{q}C₆H₄R^{S} (where R^{S} is H; F; Cl; CF₃; lower alkyl; lower alkenyl; or lower alkoxy);
R¹⁰ is lower alkyl; lower alkenyl; —(CH₂)ₒR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H is or lower alkyl); —(CH₂)ₒNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴(where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower allyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R¹¹ is H; lower allyl; lower alkenyl; —(CH₂)ₘOR⁵⁵ (where R⁵⁵ is lower alkyl; or lower alkenyl); —(CH₂)ₘSR⁵⁶ (where R⁵⁶ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴ (where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)ₒC₆H₄R^{S} (where R^{S} is H; F; Cl; CF₃; lower alkyl; lower alkenyl; or lower alkoxy);
R¹² is H; lower alkyl; lower alkenyl; —(CH₂)ₘOR⁵⁵ (where R⁵⁵ is lower alkyl; or lower alkenyl); —(CH₂)ₘSR⁵⁶ (where R⁵⁶ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴ (where: R²⁰ is H; or lower alkyl; R⁶⁴ is lower alkyl; or lower alkenyl); —(CH₂)ᵣCOOR⁵⁷ (where R⁵⁷ is lower alkyl; or lower alkenyl); —(CH₂)ᵣCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ᵣPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy):
R¹³ is lower alkyl; lower alkenyl; —(CH₂)_{q}OR⁵⁵ (where R⁵⁵ is lower alkyl; or lower alkenyl); —(CH₂)_{q}SR⁵⁶ (where R⁵⁶ is lower alkyl; or lower alkenyl); —(CH₂)_{q}NR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}OCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}NR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}N(R²⁰)COR⁶⁴ (where: R²⁰ is H; or lower alkyl; R⁶⁴ is lower alkyl; or lower alkenyl); —(CH₂)ₛCOOR⁵⁷ (where R⁵⁷ is lower alkyl; or lower alkenyl); —(CH₂)_{q}CONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ᵣPO(OR⁶⁰)₂ (where R⁶⁰ is lower alkyl; or lower alkenyl); —(CH₂)ᵣSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R¹⁴ is H; lower alkyl; lower alkenyl; —(CH₂)ₘOR⁵⁵ (where R⁵⁵ is lower alkyl; or lower alkenyl); —(CH₂)ₘSR⁵⁶ (where R⁵⁶ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower lower alkyl; R³³ is H; or lower alkyl; or lower alkenyl is R⁸²: H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); (CH₂)ₘN)(R²⁰)COR⁶⁴ (where: R²⁰ is H; lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R¹⁵ is lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where R²⁰ is H; or lower alkyl; R⁶⁴ is lower alkyl; or lower alkenyl); —NR²⁰COlower alkyl (R²⁰═H; or lower alkyl); being particularly favoured; —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or (CH₂)_{q}C₆H₄R^{S} (where R^{S} is H; F; Cl; CF₃; lower alkyl; lower alkenyl; or lower alkoxy);
R¹⁶ is lower alkyl; lower alkenyl; —(CH₂)₆OR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy); and
R¹⁷ is lower alkyl; lower alkenyl; —(CH₂)_{q}OR⁵⁵ (where R⁵⁵ is lower alkyl; or lower alkenyl); —(CH₂)_{q}SR⁵⁶ (where R⁵⁶ is lower alkyl; or lower alkenyl); —(CH₂)_{q}NR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower allyl); —(CH₂)_{q}OCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}NR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)_{q}N(R²⁰)COR⁶⁴(where: R²⁰ is H; or lower alkyl; R⁶⁴ is lower alkyl; or lower alkenyl); —(CH₂)ᵣCOOR⁵⁷ (where R⁵⁷ is lower alkyl; or lower alkenyl); —(CH₂)_{q}CONR⁵⁸R⁵⁹ (where R⁵⁸ is lower alkyl; or lower alkenyl; and R⁵⁹ is H; lower alkyl; or R⁵⁸ and R⁵⁹ taken together are-(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ᵣPO(OR⁶⁰)₂ (where R⁶⁰ is lower alkyl; or lower alkenyl); —(CH₂)ᵣSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy).

4. Compounds according to claim 2 wherein A is a group of one of the formulae A5 (with R² being H); A8; A22; A25; A38 (with R² being H); A42; and A50 and A105.

5. Compounds according to claim 4 wherein A is a group of formula 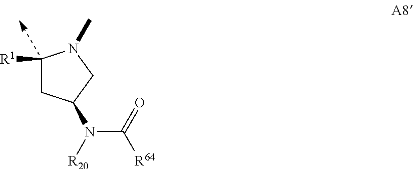
wherein R²⁰ is H or lower alkyl; and R⁶⁴ is alkyl; alkenyl; —[(CH₂)ᵤ—X]ₜ—CH₃, wherein X is —O—; —NR²⁰—, —S—; u=1-3, t=1-6, aryl; aryl-lower alkyl; or heteroaryl-lower alkyl.

6. Compounds according to claim 5 wherein R⁶⁴ is n-hexyl; n-heptyl; 4-(phenyl)benzyl; diphenylmethyl, 3-amino-propyl; 5-amino-pentyl; methyl; ethyl; isopropyl; isobutyl; n-propyl; cyclohexyl; cyclohexylmethyl; n-butyl; phenyl; benzyl; (3-indolyl)methyl; 2-(3-indolyl)ethyl; (4-phenyl)phenyl; n-nonyl; CH₃—OCH₂CH₂—OCH₂— or CH₃—(OCH₂CH₂)₂—OCH₂—.

7. Compounds according to claim 2 wherein A is a group of one of the formulae A70 to A104;
R²⁰ is H; or lower alkyl;
R¹⁸ is lower alkyl;
R¹⁹ is lower alkyl; lower alkenyl; —(CH₂)ₚOR⁵⁵ (where R⁵⁵ is lower alkyl; or lower alkenyl); —(CH₂)ₚSR⁵⁶ (where R⁵⁶ is lower alkyl; or lower alkenyl); —(CH₂)ₚNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower allyl); —(CH₂)ₚOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚN(R²⁰)COR⁶⁴ (where: R²⁰ is H; or lower alkyl; R⁶⁴ is lower alkyl; or lower alkenyl); (CH₂)ₚCOOR⁵⁷ (where R⁵⁷: lower alkyl; or lower alkenyl); (CH₂)ₚCONR⁵⁸R⁵⁹ (where R⁵⁸ is lower alkyl; or lower alkenyl; and R⁵⁹ is H; or lower alkyl; or R⁵⁸ and R⁵⁹ taken together are —(CH₂)₂₋₆—; —(CH₂)₂—O—(CH₂)₂—; —(CH₂)₂S(CH₂)₂—; or —(CH₂)₂NR⁵⁷(CH₂)₂—; where R⁵⁷ is H; or lower alkyl); —(CH₂)₂PO(OR⁶⁰)₂ (where R⁶⁰ is lower alkyl; or lower alkenyl); —(CH₂)ₚSO₂R⁶² (where R⁶² is lower alkyl; or lower alkenyl); or (CH₂)ₒC₆H₄R^{S} (where R^{S} is H; F; Cl; CF₃; lower alkyl; lower alkenyl; or lower alkoxy);
R²¹ is H; lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R²² is lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴(where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF; lower alkyl; lower alkenyl; or lower alkoxy);
R²³ is H; lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where: R²⁰ is H; or lower alkyl; R⁶⁴ is lower alkyl; or lower alkenyl); —NR²⁰COlower alkyl (R²⁰═H; or lower alkyl) being particularly favoured; —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are-(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R²⁴ is lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₇S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where: R²⁰ is H; or lower alkyl; R⁶⁴ is lower alkyl; or lower alkenyl); —NR²⁰COlower alkyl (R²⁰═H; or lower alkyl) being particularly favoured; —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R²⁵ is H; lower alkyl; lower alkenyl; —(CH₂)ₘOR⁵⁵ (where R⁵⁵ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R²⁶ is H; lower alkyl; lower alkenyl; —(CH₂)ₘOR⁵⁵ (where R⁵⁵ is lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy); or, alternatively, R²⁵ and R²⁶ taken together are —(CH₂)₂₋₆—; —(CH₂)₂O(CH₂)₂—; —(CH₂)₂S(CH₂)₂—; or —(CH₂)₂NR³⁴(CH₂)₂—;
R²⁷ is H; lower alkyl; lower alkenyl; —(CH₂)ₒOR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰ CONR³³R⁸² (where R²⁰ is H; or lower lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴ (where R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy);
R²⁸ is lower alkyl; lower alkenyl; —(CH₂)ₒR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴(where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy); and
R²⁹ is lower alkyl; lower alkenyl; —(CH₂)ₒR⁵⁵ (where R⁵⁵ is lower alkyl; or lower alkenyl); —(CH₂)ₒSR⁵⁶ (where R⁵⁶ is lower alkyl; or lower alkenyl); —(CH₂)ₒNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒN(R²⁰)COR⁶⁴(where: R²⁰ is H; or lower alkyl; R⁶⁴ is lower alkyl; or lower alkenyl); —NR²⁰COlower-alkyl (R²⁰═H; or lower alkyl) being particularly favoured; —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)_{q}C₆H₄R^{S} (where R^{S} is H; F; Cl; CF₃; lower alkyl; lower alkenyl; or lower alkoxy).

8. Compounds according to claim 7 wherein R²³, R²⁴ and R²⁹ are —NR²⁰—CO-lower alkyl where R²⁰ is H; or lower alkyl.

9. Compounds according to claim 7 or 8 wherein A is a group of one of the formulae A74 (with R²² being H); A75; A76; A77 (with R²² being H); A78; and A79.

10. Compounds according to claim 2 wherein B is a group of formula —NR²⁰CH(R⁷¹)— or an enantiomer of one of the groups A5 (with R² being H); A8; A22; A25; A38 (with R² being H); A42; A47; and A50.

11. Compounds according to claim 10 wherein B—CO is Ala; Arg; Asn; Cys; Gln; Gly; His; Ile; Leu; Lys; Met; Phe; Pro; Pro(4-NHCOPhe), Ser; Thr; Trp; Tyr; Val; Cit; Orn; tBuA; Sar; t-BuG; 4AmPhe; 3AmPhe; 2AmPhe; Phe(mC(NH₂)═NH; Phe(pC(NH₂)═NH; Phe(mNHC(NH₂)═NH; Phe(pNHC(NH₂)═NH; Phg; Cha; C₄al; C₅al; Nle; 2-Nal; 1-Nal; 4Cl-Phe; 3Cl-Phe; 2Cl-Phe; 3,4Cl₂Phe; 4F-Phe; 3F-Phe; 2F-Phe; Tic; Thi; Tza; Mso; AcLys; Dpr; A₂Bu; Dbu; Abu; Aha; Aib; Y(Bzl); Bip; S(Bzl); T(Bzl); hCha; hCys; hSer, hArg; hPhe; Bpa; Pip; OctG; MePhe; MeNle; MeAla; MeIle; MeVal; or MeLeu.

12. Compounds according to claim 10 wherein B is a group, having (L)-configuration, of formula 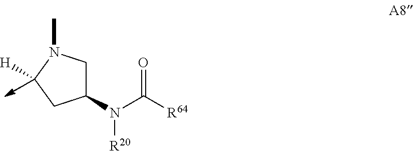
wherein R²⁰ is H; or lower alkyl; and R⁶⁴ is alkyl; alkenyl; —[(CH₂)ᵤ—X]ₜ—CH₃, wherein X is —O—, —NR²⁰—, —S—; u=1-3, t=1-6; aryl; aryl-lower alkyl; or heteroaryl-lower alkyl.

13. Compounds according to claim 12 wherein R⁶⁴ is n-hexyl; n-heptyl; 4-(phenyl)benzyl; diphenylmethyl, 3-amino-propyl; 5-amino-pentyl; methyl; ethyl; isopropyl; isobutyl; n-propyl; cyclohexyl; cyclohexylmethyl; n-butyl; phenyl; benzyl; (3-indolyl)methyl; 2-(3-indolyl)ethyl; (4-phenyl)phenyl; n-nonyl; CH₃—OCH₂CH₂—OCH₂— or CH₃—(OCH₂CH₂)₂—OCH₂—.

14. Compounds according to claim 1 or 2 wherein 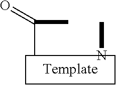
is a group of formula (b1) to (c1);
R¹ is H; or lower alkyl;
R²⁰ is H; or lower alkyl;
R³⁰ is H; or methyl;
R³¹ is H; lower allyl; lower alkenyl; —(CH₂)ₚOR⁵⁵ (where R⁵⁵ is lower alkyl; or lower alkenyl); —(CH₂)ₚNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower allyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alkyl; or lower alkenyl); or —(CH₂)ᵣC₆H₄R^{S} (where R^{S} is H; F; Cl; CF₃; lower alkyl; lower alkenyl; or lower alkoxy); most preferably —CH₂CONR⁵⁸R⁵⁹ (where R⁵⁸ is H; or lower alkyl; and R⁵⁹ is lower alkyl; or lower alkenyl);
R³² is H; or methyl;
R³³ is lower alkyl; lower alkenyl; —(CH₂)ₘOR⁵⁵ (where R⁵⁵ is lower alkyl; or lower alkenyl); (CH₂)ₘNR³⁴R⁶³ (where R³⁴ is lower alkyl; or lower alkenyl; R⁶³ is H; or lower alkyl; or R³⁴ and R⁶³ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR⁷⁵R⁸² (where R⁷⁵ is lower alkyl; or lower alkenyl; R⁸² is H; or lower alkyl; or R⁷⁵ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR⁷⁸R⁸² (where R²⁰ is H; or lower alkyl; R⁷⁸ is H; or lower alkyl; or lower alkenyl; R⁸² is H; or lower alkyl; or R⁷⁸ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷: H; or lower alkyl);
R³⁴ is H; or lower alkyl;
R³⁵: is H; lower alkyl; lower alkenyl; (CH₂)ₘOR⁵⁵ (where R⁵⁵: lower alkyl; or lower alkenyl); —(CH₂)ₘNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘOCONR³³R⁷⁵ (where R³³ is H; or lower alkyl; or lower alkenyl; R⁷⁵ is lower alkyl; or R³³ and R⁷⁵ taken together are (CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₘN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl; or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl);
R³⁶: lower alkyl; lower alkenyl; or aryl-lower alkyl;
R³⁷ is H; lower alkyl; lower alkenyl; —(CH₂)ₚOR⁵⁵ (where R⁵⁵ is lower alkyl; or lower alkenyl); —(CH₂)ₚNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); £—(CH₂)ₚNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower alky; or lower alkenyl); or —(CH₂)_{q}C₆H₄R^{S} (where R^{S} is H; F; Cl; CF₃; lower alkyl; lower alkenyl; or lower alkoxy); and
R³⁸ is H; lower alkyl; lower alkenyl; —(CH₂)ₚOR⁵⁵ (where R⁵⁵ is lower alkyl; or lower alkenyl); —(CH₂)ₚNR³³R³⁴ (where R³³ is lower alkyl; or lower alkenyl; R³⁴ is H; or lower alkyl; or R³³ and R³⁴ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚOCONR³³R⁷⁵ (where R³³ is H; or lower alkyl; or lower alkenyl; R⁷⁵ is lower alkyl; or R³³ and R⁷⁵ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚNR²⁰CONR³³R⁸² (where R²⁰ is H; or lower alkyl; R³³ is H; or lower alkyl; or lower alkenyl; R⁸² is H; or lower alkyl; or R³³ and R⁸² taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₚN(R²⁰)COR⁶⁴ (where: R²⁰ is H; or lower alkyl; R⁶⁴ is lower alkyl; or lower alkenyl); —(CH₂)ₒCOOR⁵⁷ (where R⁵⁷ is lower alkyl; or lower alkenyl); —(CH₂)ₒCONR⁵⁸R⁵⁹ (where R⁵⁸ is lower alkyl, or lower alkenyl; and R⁵⁹ is H; lower alkyl; or R⁵⁸ and R⁵⁹ taken together are —(CH₂)₂₋₆—; —(CH₂)₂O(CH₂)₂—; —(CH₂)₂S(CH₂)₂—; or —(CH₂)₂NR⁵⁷(CH₂)₂—; where R⁵⁷ is H; or lower alkyl); —(CH₂)ₒPO(OR⁶⁰)₂ (where R⁶⁰ is lower alkyl; or lower alkenyl); —(CH₂)ₒSO₂R⁶² (where R⁶² is lower allyl; or lower alkenyl); or —(CH₂)_{q}C₆H₄R^{S} (where R^{S} is H; F; Cl; CF₃; lower alkyl; lower alkenyl; or lower alkoxy).

15. Compounds according to claim 14 wherein R¹ is H; R²⁰ is H; R³⁰ is H; R³¹ is carboxymethyl; or lower alkoxycarbonylmethyl; R³² is H; R³⁵ is methyl; R³⁶ is methoxy; R³⁷ is H and R³⁸ is H.

16. Compounds according to claim 2 wherein the template is ^{D}Pro-^{L}Pro, ^{D}Pro-4Hyp2, ^{D}Pro-Oic, ^{D}Pro-4 Mp1, ^{D}Ser-^{L}Pro, ^{D}4Hyp2-^{L}Pro or ^{D}Glu-^{L}Pro or, instead of said residues of ^{D}Pro and/or ^{L}′Pro, contains certain substituted derivatives thereof with substitution patterns as shown in formulae A8′ and A8″ given in claim 5 and, respectively, claim 12.

17. Compounds according to claim 1 wherein the α-amino acid residues in positions 1 to 8 in the chain Z are:
P1: of type C, or of type D, or of type F;
P2: of type C, or of type F;
P3: of type C, or of type D;
P4: of type C, or of type D, or of type F, or the residue is Gly;
P5: of type C, or of type D, or of type E, or of type F, or the residue is Gly;
P6: of type C, or of type D;
P7: of type C, or of type D, or of type F;
P8: of type C, or of type D, of type F; or
P2 and P7, taken together, can form a group of type H;
at P4 and P5 also D-isomers being possible.

18. Compounds according to claim 17 wherein the α-amino acid residues in positions 1 to 8 of the chain Z are:
P1: Phe, Glu, Cha, Met, Asp;
P2: Glu, Thr, Ala, Leu, Cys;
P3: Trp(6C1), Trp, Ala, Phe, Tyr;
P4: Leu, Gly, Tyr, Cys, Trp, ^{D}Trp;
P5: Ala, ^{D}Ala, Gly, Tyr, Asp, Lys, Orn;
P6: Trp, OctG, Ala, Tyr;
P7: Glu, Ala, Tyr, Leu, Cys;
P8: Phe, Trp, Glu(cHx), Ile, Met, Glu, Cha, Leu, Val; and
Cys if present at P2 and P7 can form a disulfide bridge.

19. Compounds according to claim 1 wherein the α-amino acid residues in positions 1 to 8 in the chain Z are:
P1: of type F;
P2: of type F;
P3: of type D;
P4: of type D;
P5: of type E;
P6: of type D;
P7: of type F;
P8: of type C; or
P2 and P7, taken together, form a group of type H;
at P4 D-isomers being possible.

20. Compounds according to claim 19 wherein the α-amino acid residues in positions 1 to 8 are:
P1: Asp;
P2: Cys;
P3: Phe, Tyr;
P4: Trp, ^{D}Trp;
P5: Lys, Orn;
P6: Tyr;
P7: Cys,
P8: Cha, Leu, Val; and
Cys at P2 and P7 can form a disulfide bridge.

21. Compounds according to claim 1 wherein the α-amino acid residues in positions 1 to 8 in the chain Z are:
P1: of type C, or of type D, or of type F;
P2: of type C, or of type F;
P3: or of type C, of type D;
P4: of type C, or of type D, or of type F, or the residue is Gly;
P5: of type C, or of type D, or of type F, or the residue is Gly;
P6: of type C, or of type D;
P7: of type C, or of type D, or of type F;
P8: of type C, or of type D, or of type F; or
P2 and P7, taken together, form a group of type H;
at P5 also D-isomers being possible.

22. Compounds according to claim 21 wherein the α-amino acid residues in positions 1 to 8 are:
P1: Phe, Glu, Cha, Met;
P2: Glu, Thr, Ala, Leu;
P3: Trp(6Cl), Trp, Ala;
P4: Leu, Gly, Tyr, Cys;
P5: Ala, ^{D}Ala, Gly, Tyr, Asp;
P6: Trp, OctG, Ala;
P7: Glu, Ala, Tyr, Leu; and
P8: Phe, Trp, Glu(cHx), Ile, Met, Glu, Cha.

23. A compound of formula I according to claim 1 wherein the template is ^{D}Pro-^{L}Pro and the amino acid residues in position 1-8 are:
P1: Phe;
P2: Glu;
P3: Trp(6Cl);
P4: Leu;
P5: Ala;
P6: Trp;
P7: Glu; and
P8: Phe.

24. A compound of formula I according to claim 1 wherein the template is ^{D}Pro-^{L}4Hyp2 and the amino acid residues in position 1-8 are:
P1: Phe;
P2: Glu;
P3: Trp;
P4: Leu;
P5: Ala;
P6: Trp;
P7: Glu; and
P8: Phe.

25. A compound of formula I according to claim 1 wherein the template is ^{D}Pro-^{L}Pro and the amino acid residues in position 1-8 are:
P1: Phe;
P2: Glu;
P3: Trp;
P4: Leu;
P5: Ala;
P6: Trp;
P7: Glu; and
P8: Glu(cHx).

26. A compound of formula I according to claim 1 wherein the template is ^{p}Pro-^{L}Pro, and the amino acid residues in position 1-8 are:
P1: Phe;
P2: Glu;
P3: Trp;
P4: Leu;
P5: Asp;
P6: Trp;
P7: Glu; and
P8: Phe.

27. A compound of formula I according to claim 1 wherein the template is ^{D}Pro-^{L}Pro and the amino acid residues in position 1-8 are:
P1: Asp;
P2: Cys;
P3: Phe;
P4: Trp;
P5: Lys;
P6: Tyr;
P7: Cys; and
P8: Val;
Cys at P2 and P7 forming a disulfide bridge.

28. A compound of formula I according to claim 1 wherein the template is ^{D}Pro-^{L}Pro and the amino acid residues in position 1-8 are:
P1: Asp;
P2: Cys;
P3: Phe;
P4: ^{D}Trp;
P5: Orn;
P6: Tyr;
P7: Cys; and
P8: Val;
Cys at P2 and P7 forming a disulfide bridge.

29. Enantiomers of the compounds of formula I as defined in claim 1.

30. Compounds according to claim 1 for use as therapeutically active substances.

31. Compounds according to claim 30 having agonizing or antagonizing activity against urotensin II or showing inhibition of the STAT6/NCoA-1 interaction being useful for treating renal disease, diabetes, cardiovascular dysfunction, inflammation as well as allergic airways diseases like allergic rhinitis and asthma.

32. A pharmaceutical composition containing a compound according to claim 1 and a pharmaceutically inert carrier.

33. Compositions according to claim 32 in a form suitable for oral, topical, transdermal, injection, buccal, transmucosal, pulmonary or inhalation administration.

34. Compositions according to claim 32 in form of tablets, dragees, capsules, solutions, liquids, gels, plaster, creams, ointments, syrup, slurries, suspensions, spray, nebuliser or suppositories.

35. The use of compounds according to claim 1 for the manufacture of a medicament for use as an agonist or antagonist of urotensin II or an inhibitor of the STAT6/NCoA-1 interaction.

36. The use according to claim 35 wherein said urotensin II agonizing or antagonizing or STAT6/NCoA-1 interaction inhibiting medicament is intended to be used in cases where renal disease is mediated or resulting from, or where diabetes is mediated or resulting from, or where cardiovascular dysfunction is mediated or resulting from, or where inflammation is mediated or resulting from urotensin II activity; or where allergic airways diseases like allergic rhinitis and asthma are mediated or resulting from the STAT6/NCoA-1 interaction.

37. A process for the manufacture of compounds according to claim 1 which process comprises
(a) coupling an appropriately functionalized solid support with an appropriately N-protected derivative of that amino acid which in the desired end-product is in positions 3, 4 or 5, any functional group which may be present in said N-protected amino acid derivative being likewise appropriately protected;
(b) removing the N-protecting group from the product thus obtained;
(c) coupling the product thus obtained with an appropriately N-protected derivative of that amino acid which in the desired end-product is one position nearer the N-terminal amino acid residue, any functional group which may be present in said N-protected amino acid derivative being likewise appropriately protected;
(d) removing the N-protecting group from the product thus obtained;
(e) repeating steps (c) and (d) until the N-terminal amino acid residue has been introduced;
(f) coupling the product thus obtained with a compound of the general formula 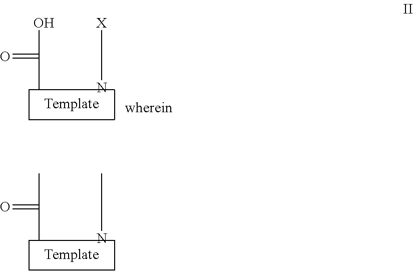
is as defined in claim 1 and X is an N-protecting group or, alternatively, if 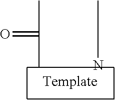
is to be group (a1), (a2) or (a3) above,
(fa) coupling the product obtained in step (e) with an appropriately N-protected derivative of an amino acid of the general formula
HOOC—B—H III
or
HOOC-A-H IV
wherein B and A are as defined in claim 1, any functional group which may be present in said N-protected amino acid derivative being likewise appropriately protected;
(fb) removing the N-protecting group from the product thus obtained; and
(fc) coupling the product thus obtained with an appropriately N-protected derivative of an amino acid of the above general formula IV or formula
HOOC—B3-H V
wherein B3 is as defined in claim 1
and, respectively, formula III, any functional group which may be present in said N-protected amino acid derivative being likewise appropriately protected;
(g) removing the N-protecting group from the product obtained in step (f) or (fc);
(h) coupling the product thus obtained with an appropriately N-protected derivative of that amino acid which in the desired end-product is in position, any functional group which may be present in said N-protected amino acid derivative being likewise appropriately protected;
(i) removing the N-protecting group from the product thus obtained;
(j) coupling the product thus obtained with an appropriately N-protected derivative of that amino acid which in the desired end-product is one position farther away from position 8, any functional group which may be present in said N-protected amino acid derivative being likewise appropriately protected;
(k) removing the N-protecting group from the product thus obtained;
(l) repeating steps (j) and (k) until all amino acid residues have been introduced;
(m) if desired, selectively deprotecting one or several protected functional group(s) present in the molecule and appropriately substituting the reactive group(s) thus liberated;
(n) if desired, forming an interstrand linkage between side-chains of appropriate amino acid residues at positions 2 and 7;
(o) detaching the product thus obtained from the solid support;
(p) cyclizing the product cleaved from the solid support;
(q) removing any protecting groups present on functional groups of any members of the chain of amino acid residues and, if desired, any protecting group(s) which may in addition be present in the molecule; and
(r) if desired, converting the product thus obtained into a pharmaceutically acceptable salt or converting a pharmaceutically acceptable, or unacceptable, salt thus obtained into the corresponding free compound of formula I or into a different, pharmaceutically acceptable, salt.

38. A process for the manufacture of compounds according to claim 1 which process comprises
(a′) coupling an appropriately functionalized solid support with a compound of the general formula 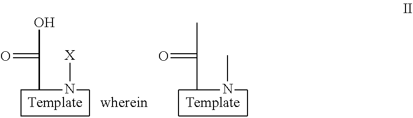
is as defined in claim 1 and X is an N-protecting group or, alternatively, if 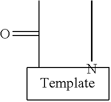
is to be group (a1), (a2) or (a3) above,
(a′a) coupling said appropriately functionalized solid support with an appropriately
N-protected derivative of an amino acid of the general formula
HOOC—B—H III
or
HOOC-A-H IV
wherein B and A are as defined in claim 1, any functional group which may be present in said N-protected amino acid derivative being likewise appropriately protected;
(a′b) removing the N-protecting group from the product thus obtained; and
(a′c) coupling the product thus obtained with an appropriately N-protected derivative of an amino acid of the above general formula IV or formula
HOOC—B3-H V
wherein B3 is as defined in claim 1
and, respectively, formula III, any functional group which may be present in said N-protected amino acid derivative being likewise appropriately protected;
(b′) removing the N-protecting group from the product obtained in step (a′) or (a′c)
(c′) coupling the product thus obtained with an appropriately N-protected derivative of that amino acid which in the desired end-product is in position 8, any functional group which may be present in said N-protected amino acid derivative being likewise appropriately protected;
(d′) removing the N-protecting group from the product thus obtained;
(e′) coupling the product thus obtained with an appropriately N-protected derivative of that amino acid which in the desired end-product is one position farther away from position 8, any functional group which may be present in said N-protected amino acid derivative being likewise appropriately protected;
(f′) removing the N-protecting group from the product thus obtained;
(g′) repeating steps (e′) and (f′) until all amino acid residues have been introduced;
(h′) if desired, selectively deprotecting one or several protected functional group(s) present in the molecule and appropriately substituting the reactive group(s) thus liberated;
(i′) if desired forming an interstrand linkage between side-chains of appropriate amino acid residues at opposite positions 2 and 7;
(j′) detaching the product thus obtained from the solid support;
(k′) cyclizing the product cleaved from the solid support;
(l′) removing any protecting groups present on functional groups of any members of the chain of amino acid residues and, if desired, any protecting group(s) which may in addition be present in the molecule; and
(m′) if desired, converting the product thus obtained into a pharmaceutically acceptable salt or converting a pharmaceutically acceptable, or unacceptable, salt thus obtained into the corresponding free compound of formula I or into a different, pharmaceutically acceptable, salt.

39. A modification of the processes according to claim 37 for the manufacture of compounds according to claim 29 in which enantiomers of all chiral starting materials are used.
